SEQUENCE LISTING (14 sequences)

SEQ ID NO: 1 - protein, 124 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQ

SEQ ID NO: 2 - protein, 66 aa, Homo sapiens
MAEPRQEFEVMEDHAGTYGLGDRKDQGGYTMHQDQEGDTDAGLKAEEAGIGDTPSLEDEAAGHVTQ

SEQ ID NO: 3 - protein, 66 aa, Homo sapiens; identical to SEQ ID NO: 2

SEQ ID NO: 4 - protein, 95 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQ

SEQ ID NO: 5 - protein, 240 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPA

SEQ ID NO: 6 - protein, 240 aa, Homo sapiens; identical to SEQ ID NO: 5

SEQ ID NO: 7 - DNA, 21 bp, Artificial Sequence
  CCLS5 forward primer
gggtaccatgaagatctctgc

SEQ ID NO: 8 - DNA, 21 bp, Artificial Sequence
  CCLS5 Reverse primer
gcgagggagaggtaggcaaag

SEQ ID NO: 9 - DNA, 18 bp, Artificial Sequence
  IL-1P forward primer
gagcaccttcttttcctt

SEQ ID NO: 10 - DNA, 23 bp, Artificial Sequence
  IL-1P Reverse primer
cacacaccagcaggttatcatca

SEQ ID NO: 11 - DNA, 21 bp, Artificial Sequence
  CCL2  forward primer
ggctcagccagatgcagttaa

SEQ ID NO: 12 - DNA, 24 bp, Artificial Sequence
  CCL2  Reverse primer
cctactcattgggatcatcttgct

SEQ ID NO: 13 - DNA, 20 bp, Artificial Sequence
  GAPDH forward primer
tgtgtccgtcgtggatctga

SEQ ID NO: 14 - DNA, 21 bp, Artificial Sequence
  GAPDH Reverse primer
cctgcttcaccaccttcttga

(19) United States
(12) Patent Application Publication
(10) Pub. No.: US 2024/0201204 A1
(43) Pub. Date: Jun. 20, 2024

(54) Biomarker and Druggable Target of Neurodegeneration

(71) Applicant: University Hospitals Cleveland Medical Center, Cleveland, OH (US)

(72) Inventors: Andrew A. Pieper, Cleveland, OH (US); Min-Kyoo Shin, Cleveland, OH (US); Edwin Vazquez-Rosa, Cleveland, OH (US)

(73) Assignee: University Hospitals Cleveland Medical Center, Cleveland, OH (US)

(21) Appl. No.: 17/790,480

(22) PCT Filed: Jan. 7, 2021
(86) PCT No.: PCT/US21/12511

§ 371 Date: Jun. 30, 2022

Related U.S. Application Data
(60) Provisional application No. 62/958,179, filed on Jan. 7, 2020.
(60) Provisional application No. 63/042,129, filed on Jun. 22, 2020.
(60) Provisional application No. 63/106,492, filed on Oct. 28, 2020.

(51) Int. Cl. G01N 33/68; A61K 31/335; A61K 31/403; A61K 31/603

(52) U.S. Cl. CPC G01N 33/6896; A61K 31/335; A61K 31/403; A61K 31/603; G01N 33/6854; G01N 2333/4709; G01N 2800/52

(57) ABSTRACT

In one aspect, disclosed herein is a method of diagnosing and/or prognosing a neurodegenerative disease in a subject, the method comprising: obtaining a plasma or blood sample from a subject; and detecting a level of acetylated Tau in the plasma or blood sample, wherein a level of acetylated Tau that is at least 25% or 50% higher than a control level in a healthy subject indicates that the subject has a neurodegenerative disease such as traumatic brain injury.

### CROSS-REFERENCE TO RELATED APPLICATIONS

This application is a U.S. national phase application of PCT International Patent Application No. PCT/US2021/012511, filed Jan. 7, 2021 which claims priority to and the benefit of U.S. Provisional Application Nos. 62/958,179 filed Jan. 7, 2020, 63/042,129 filed Jun. 22, 2020 and 63/106,492 filed Oct. 28, 2020, each of which is incorporated herein by reference in its entirety.

### STATEMENT REGARDING FEDERALLY SPONSORED RESEARCH

This invention was made with government support under Grant Nos. RO1AG066707, 1 P30 AGO62428-01, NS098740, and UL1TR001412 awarded by the National Institute of Health, and 2 101 BXOO2439-04A1 and CDA-2 RX002928 awarded by the Department of Veterans Affairs; the Government has certain rights in the invention.

### REFERENCE TO SEQUENCE LISTING

This specification includes a sequence listing submitted herewith, which includes the file entitled 189172-020104_ST25.txt having the following size: 9,472 bytes which was created Sep. 8, 2023, the contents of which are incorporated by reference herein.

### BACKGROUND

A neurodegenerative disease is an umbrella term for chronic degeneration of neurons in, e.g., the central nervous system (CNS), characterized by molecular and genetic changes in nerve cells that result in nerve cell degeneration and ultimately nerve dysfunction and death (Bertram, 2005). Neurodegenerative diseases include, but are not limited to, Alzheimer's disease (AD), Amyotrophic lateral sclerosis (ALS), Huntington's disease (HD), and Parkinson's disease (PD) (Chesselet, 2003; Hyman, 1991; Howell, 2000; Ciammola, 2007; Riviere, 1998; Katoh-Semba, 2002; and The Merck Manual).

Neurodegenerative diseases currently affect millions of people worldwide, and the incidence of disease is rapidly increasing as the aging population expands. The magnitude and trend of this problem places a growing human and financial strain on healthcare systems, which is exacerbated by the absence of effective diagnostics and treatments for many of the most common afflictions. Thus, there remains a great need for biomarkers for diagnosing and/or prognosing neurodegenerative diseases, and small molecules that could prevent the death of neurons in a variety of in vivo contexts. Such biomarkers and neuroprotective agents could possess general utility for diagnosing and treating disorders associated with neuron cell death and other causes.

### SUMMARY

In one aspect, disclosed herein is a method of diagnosing and/or prognosing a neurodegenerative disease in a subject, the method comprising: obtaining a plasma or blood sample from a subject; and detecting a level of acetylated Tau in the plasma or blood sample, wherein a level of acetylated Tau that is at least 25% or 50% higher than a control level in a healthy subject indicates that the subject has a neurodegenerative disease such as traumatic brain injury.

In some embodiments, the obtaining step comprises obtaining a plasma sample from the subject. In some embodiments, the method further comprises depleting albumin and immunoglobulin from the plasma sample. In some embodiments, the method does not involve any brain biopsy sample.

In some embodiments, the detecting step comprises using an antibody or antigen-binding fragment thereof that specifically binds acetylated Tau. In some embodiments, the antibody is a polyclonal antibody. In some embodiments, the antibody is a monoclonal antibody.

In a further aspect, a method of treating a neurodegenerative disease in a subject is provided, the method comprising administering to a subject in need thereof a therapeutically effective amount of an agent that blocks GAPDH S-nitrosylation, inhibits p300/CBP activity, and/or enhances Sirtuin1 activity, whereby accumulation of ac-tau in brain and/or plasma in the subject is reduced.

In some embodiments, the method can include administering a therapeutically effective amount of an inhibitor of GAPDH nitrosylation such as CGP3466B (Omigapil) to the subject.

In some embodiments, the method can include administering a therapeutically effective amount of p300/CBP inhibitor such as salsalate and/or diflunisal to the subject. In some embodiments, the salsalate and/or diflunisal is administered at a low, non-anti-neuroinflammatory dose, wherein said dose is 50% or less than an anti-neuroinflammatory dose In some embodiments, the low, non-anti-neuroinflammatory dose is about 10-25 mg/kg/day. In some embodiments, the anti-neuroinflammatory dose is about 50 mg/kg/day.

In some embodiments, the method can further include co-administering an effective amount of 3,6-dibromo-β-fluoro-N-(3-methoxyphenyl)-9H-carbazole-9-propanamine to the subject.

In some embodiments, the method can include administering an effective amount of 3,6-dibromo-β-fluoro-N-(3-methoxyphenyl)-9H-carbazole-9-propanamine to the subject.

Another aspect relates to a method of treating a neurodegenerative disease in a subject, the method comprising administering a low, non-anti-neuroinflammatory dose of salsalate to a subject having a neurodegenerative disease, wherein said dose is 50% or less than an anti-neuroinflammatory dose. In some embodiments, the low, non-anti-neuroinflammatory dose is about 10-25 mg/kg/day. In some embodiments, the anti-neuroinflammatory dose is about 50 mg/kg/day. In some embodiments, the method further comprises co-administering an effective amount of 3,6-dibromo-β-fluoro-N-(3-methoxyphenyl)-9H-carbazole-9-propanamine to the subject. In some embodiments, the method further comprises co-administering an effective amount of CGP3466B (Omigapil) to the subject.

A further aspect relates to an apparatus for diagnosing and/or prognosing a neurodegenerative disease in a subject, comprising: a support material; and an antibody or antigen-binding fragment thereof that specifically binds acetylated Tau, wherein said antibody or antigen-binding fragment thereof is adsorbed in or associated with the support material. In some embodiments, the antibody is a polyclonal antibody. In some embodiments, the antibody is a monoclonal antibody.

Also disclosed herein are antibodies and antigen-binding fragments thereof that specifically bind acetylated Tau. Polyclonal and monoclonal antibodies can be made using methods known in the art.

In a further aspect, provided herein is a method of treating TBI, comprising administering to a patient in need thereof a therapeutically effective amount of CGP3466B/omigapil. CGP3466B/omigapil for use in the treatment of TBI is also provided.

In another aspect, provided herein is a method of treating brain and muscular deficits in congenital muscular dystrophy, or other neurogenerative diseases such as AD, comprising administering to a patient in need thereof a therapeutically effective amount of salsalate or diflunisal or other inhibitors of p300/CBP. Salsalate, diflunisal or other inhibitors of p300/CBP for use in the treatment of brain and muscular deficits in congenital muscular dystrophy, or for the treatment of other neurogenerative diseases such as AD is also provided.

In another aspect, provided herein is a method of treating brain and muscular deficits in congenital muscular dystrophy, comprising administering to a patient in need thereof a therapeutically effective amount of P7C3 compounds such as P7C3A20, or other NAD-elevating agents, or Sirt1-activators. P7C3 compounds such as P7C3A20, or other NAD-elevating agents, or Sirt1-activators for use in the treatment of brain and muscular deficits in congenital muscular dystrophy is also provided.

In another aspect, provided herein is a method of treating retinal injury and disease, such as after TBI, comprising administering to a patient in need thereof a therapeutically effective amount of CGP3466B/omigapil. CGP3466B/omigapil for use in the treatment of retinal injury and disease, such as after TBI, is also provided.

In another aspect, provided herein is a method of treating retinal injury and disease, such as after TBI, comprising administering to a patient in need thereof a therapeutically effective amount of salsalate or other inhibitors of p300/CBP. Salsalate or other inhibitors of p300/CBP for use in the treatment of retinal injury and disease, such as after TBI, is also provided.

In another aspect, provided herein is a method of treating retinal injury and disease, such as after TBI, comprising administering to a patient in need thereof a therapeutically effective amount of P7C3 compounds such as P7C3A20, or other NAD-elevating agents, or Sirt1-activators. P7C3 compounds such as P7C3A20, or other NAD-elevating agents, or Sirt1-activators for use in the treatment of retinal injury and disease, such as after TBI, is also provided.

### BRIEF DESCRIPTION OF THE DRAWINGS

FIGS. 1A-1G. Neuronal Tau Acetylation After TBI Induces Axon Initial Segment Degradation and Pathologic Tau Mislocalization.

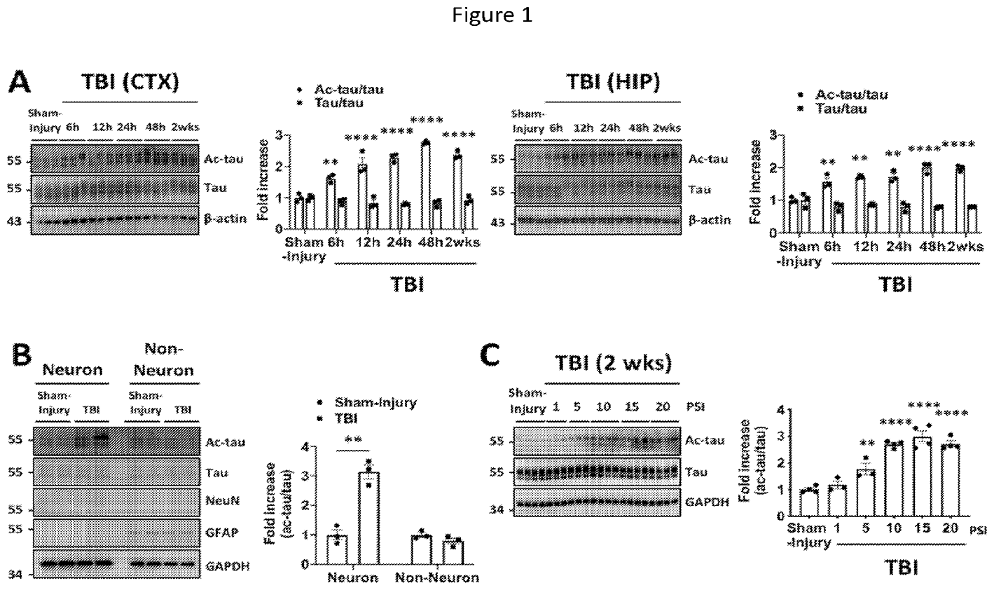

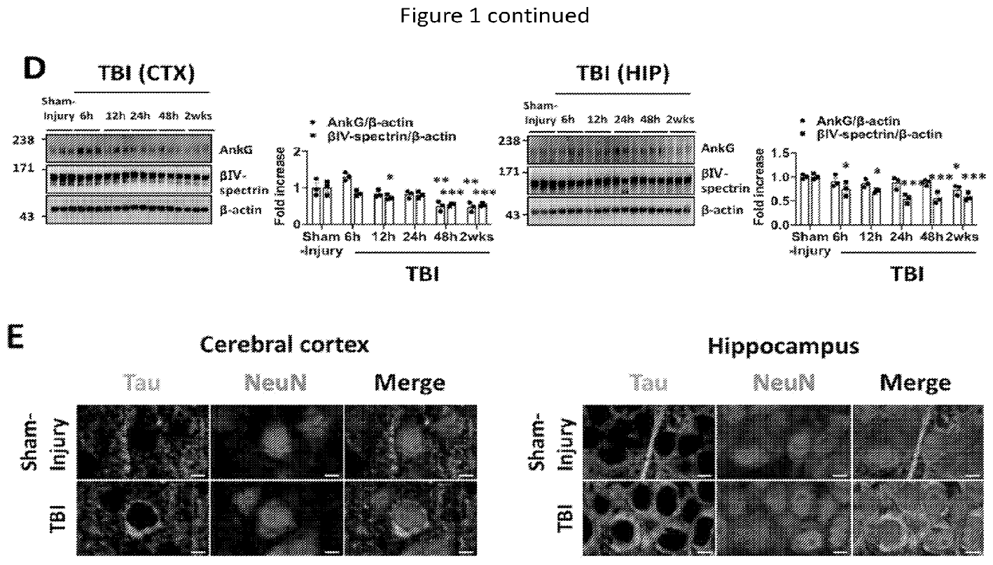

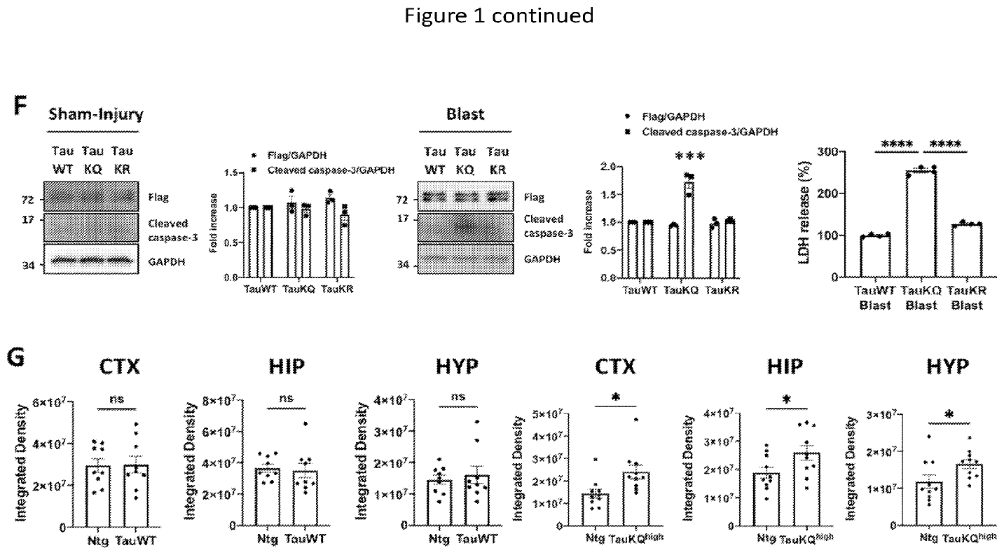

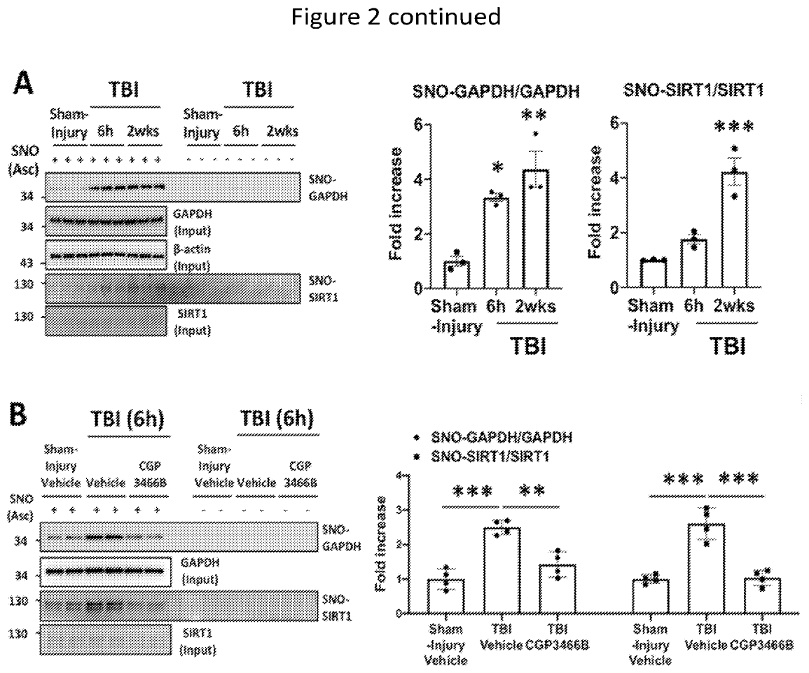

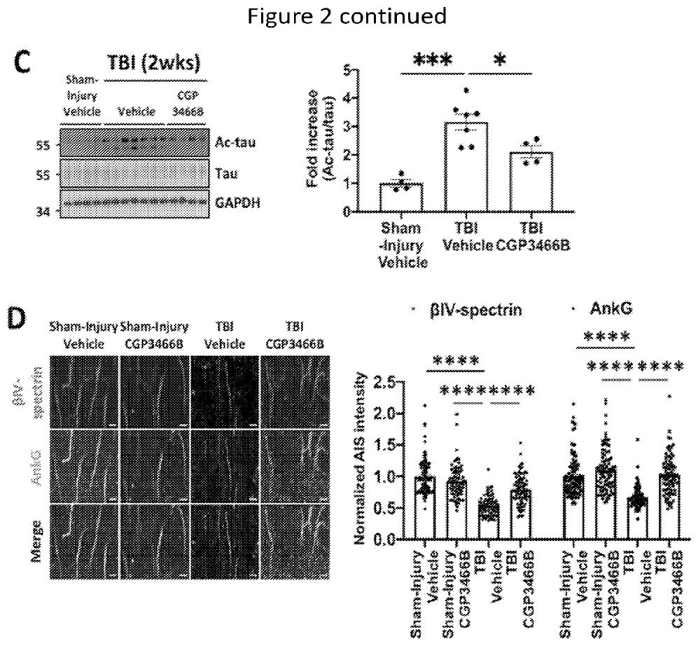

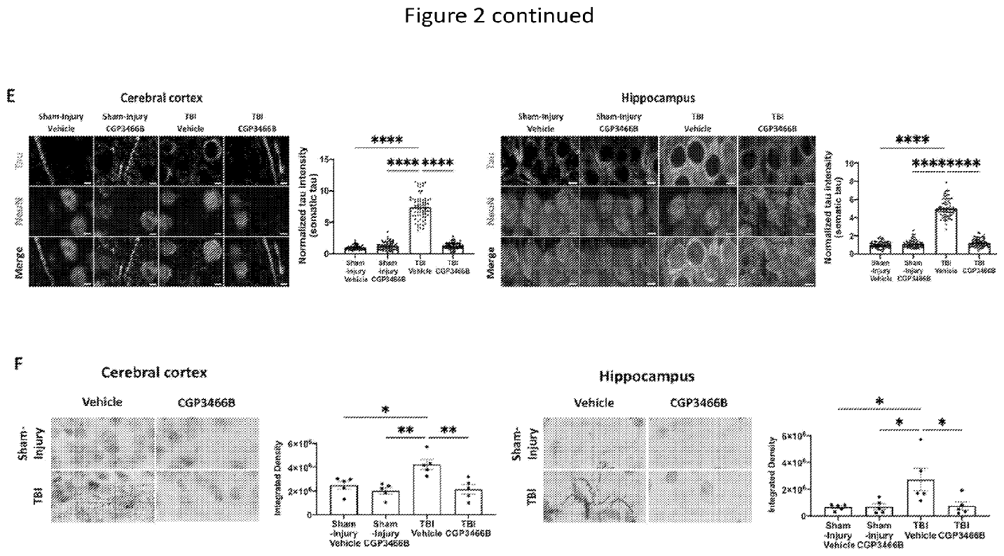

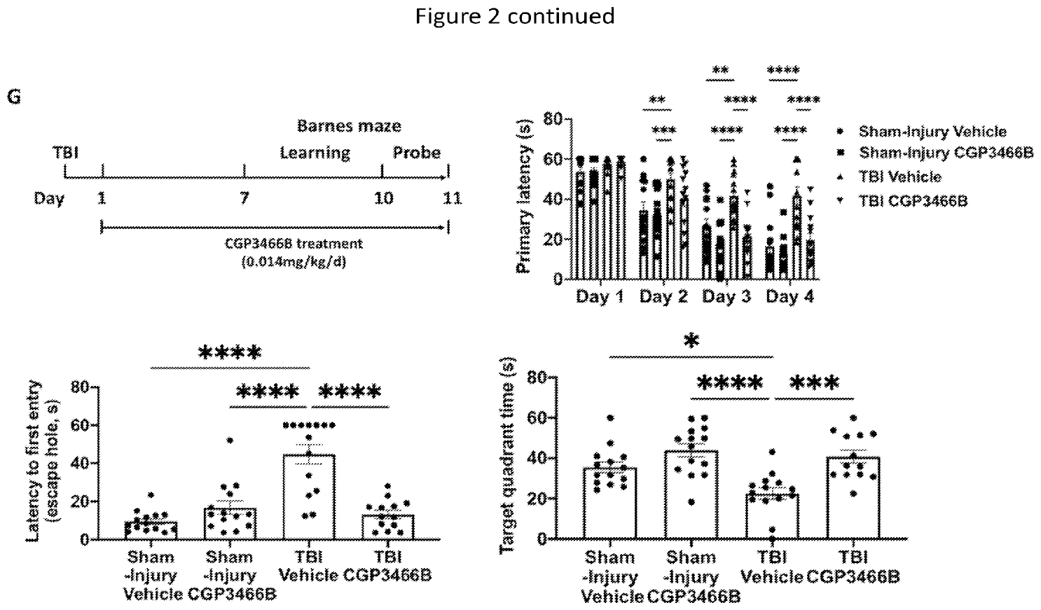

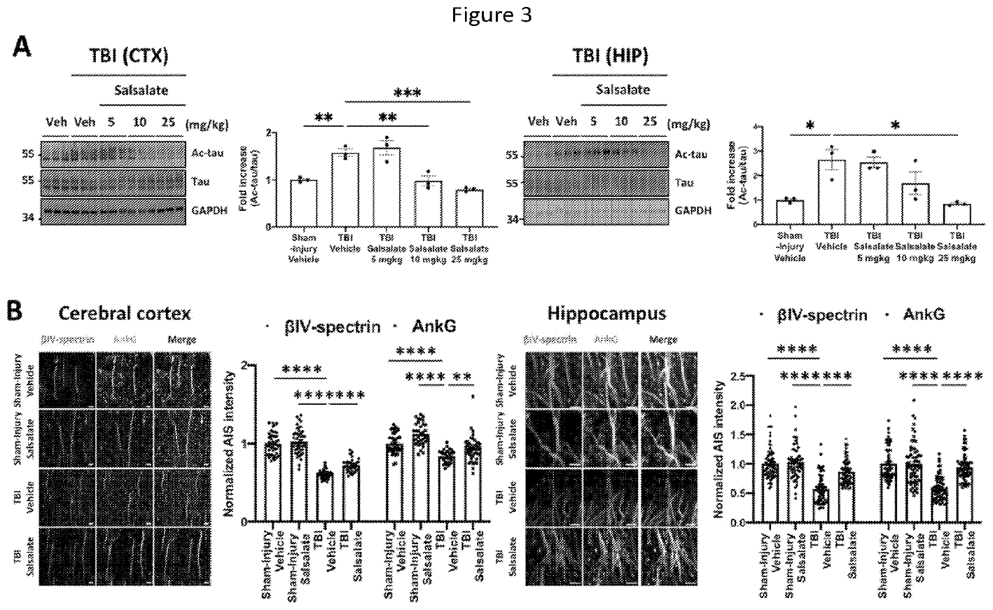

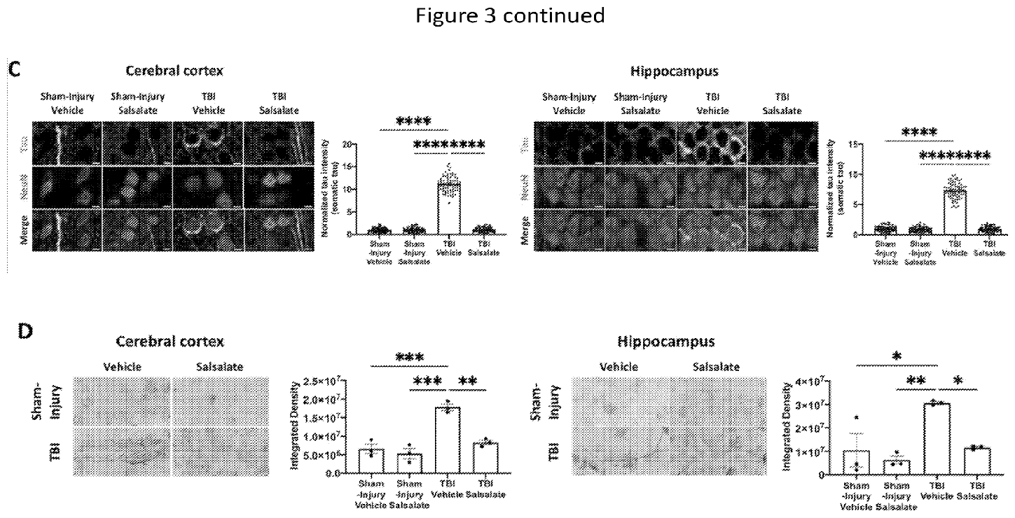

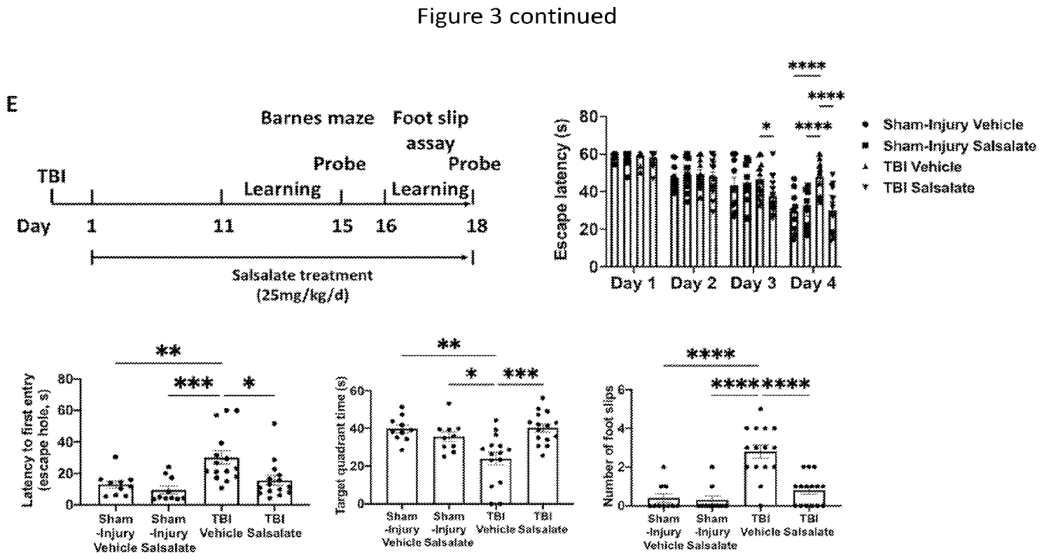

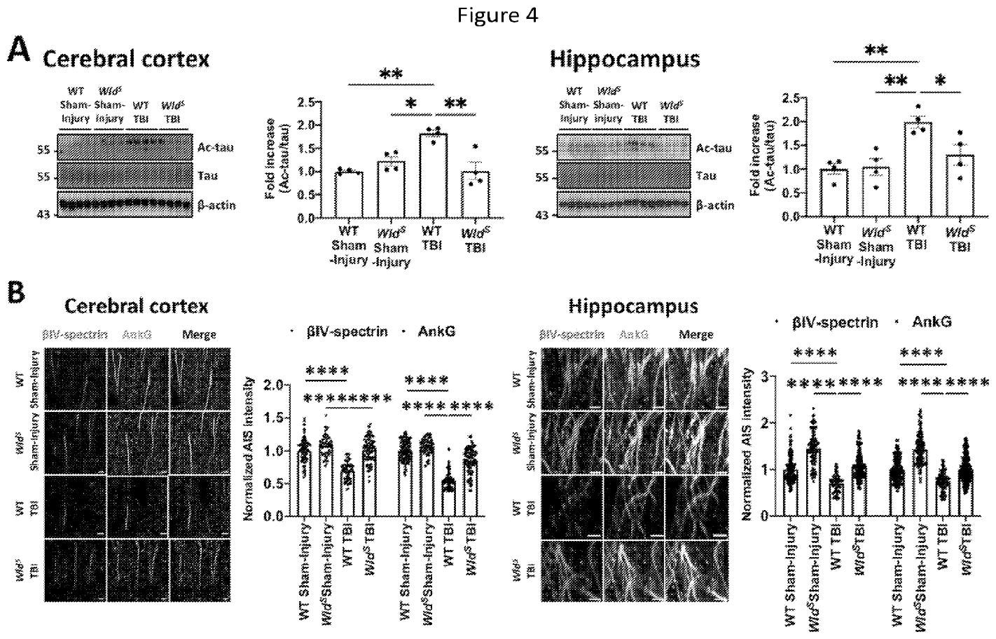

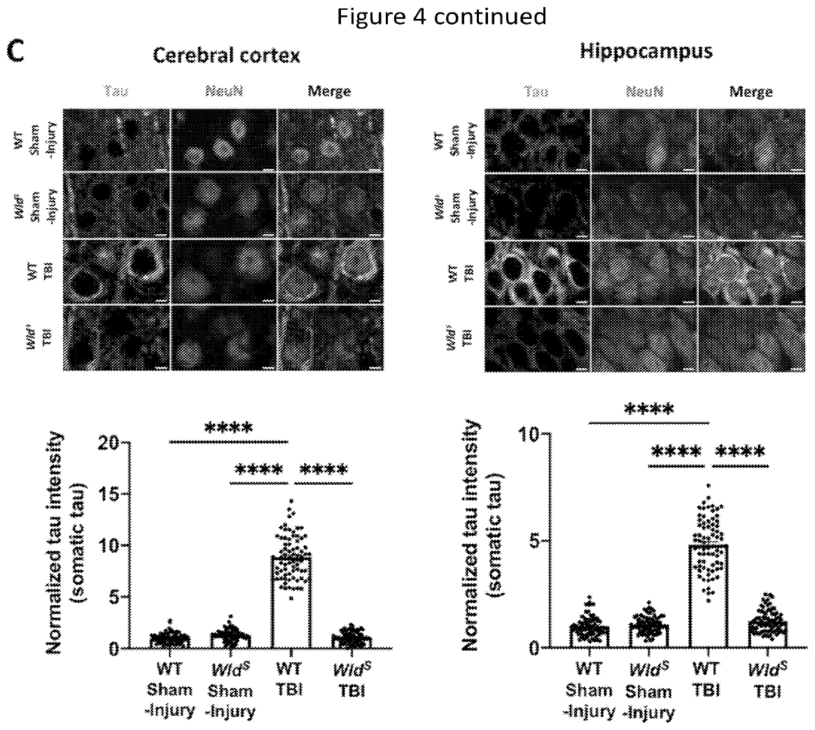

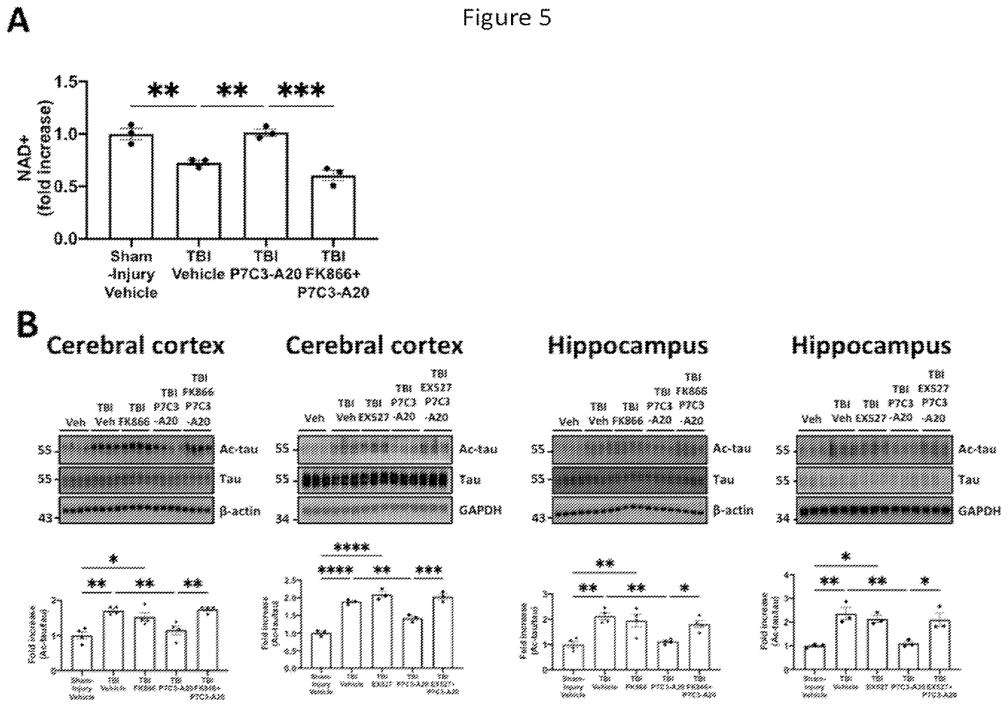

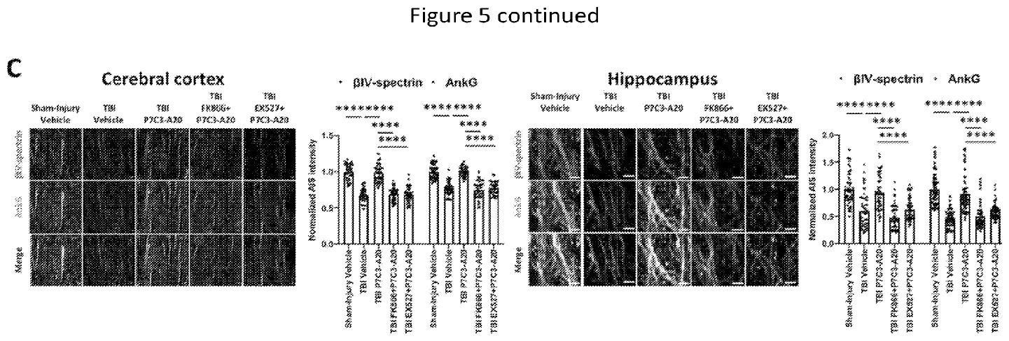

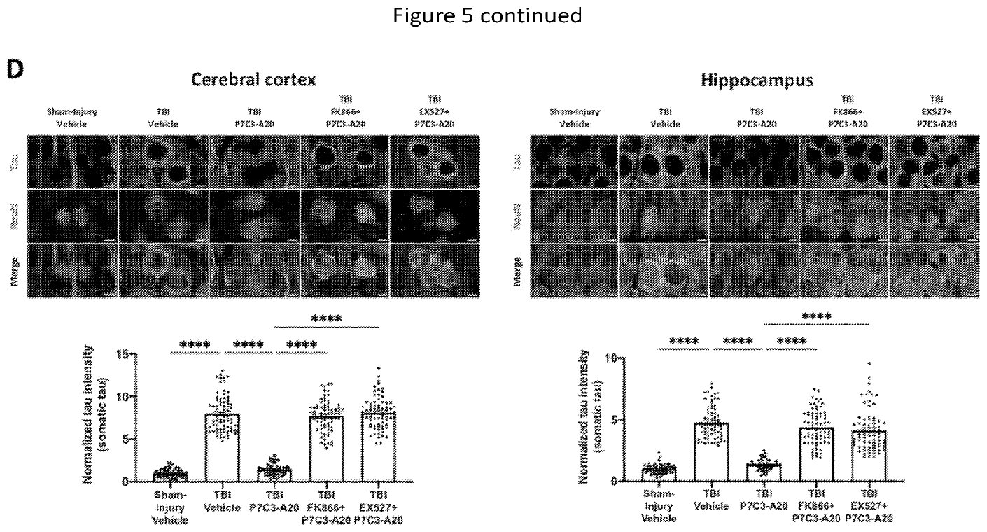

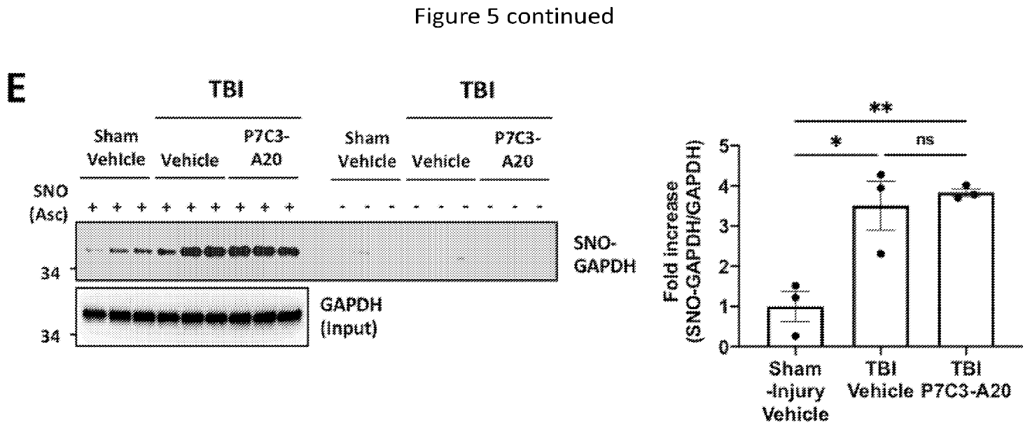

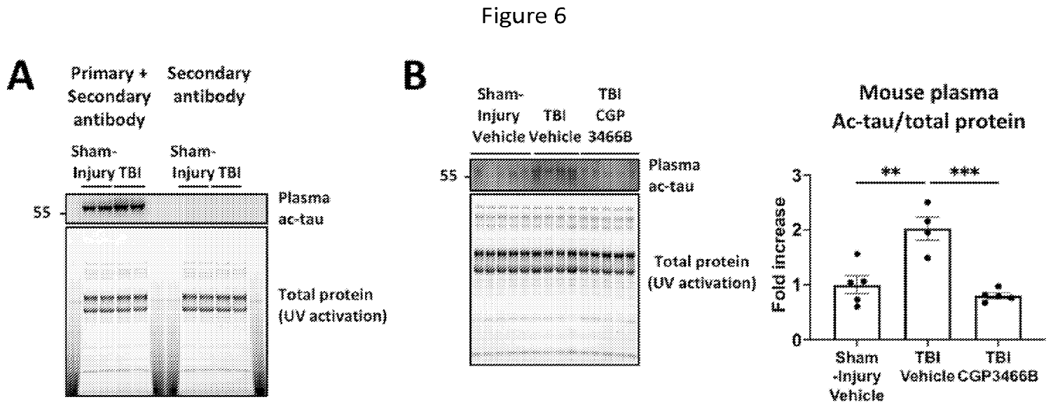

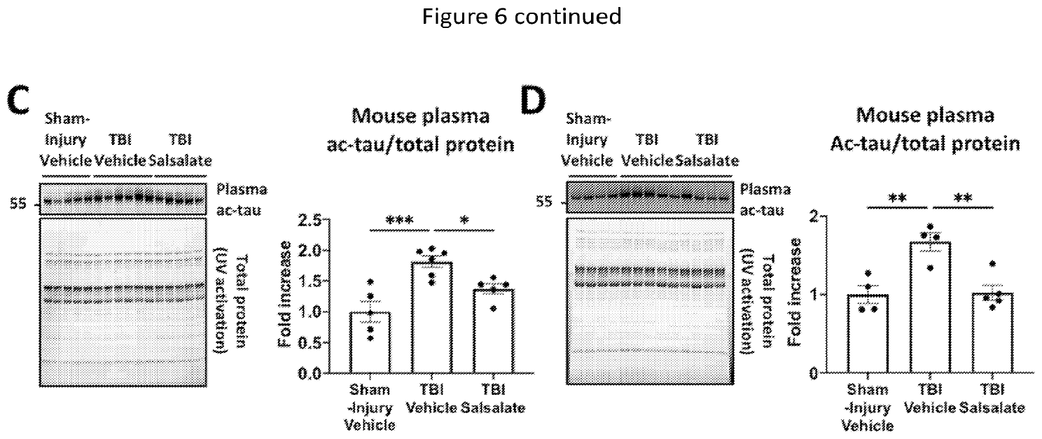

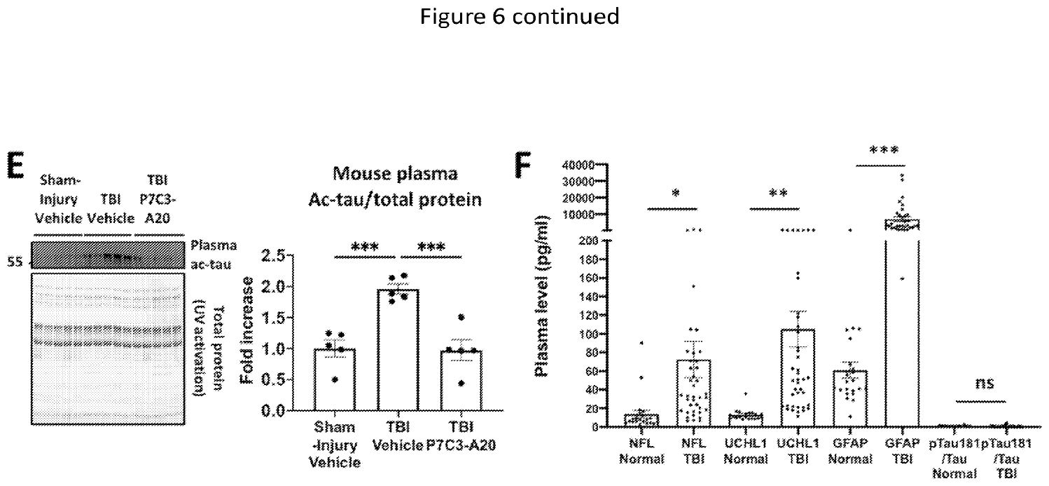

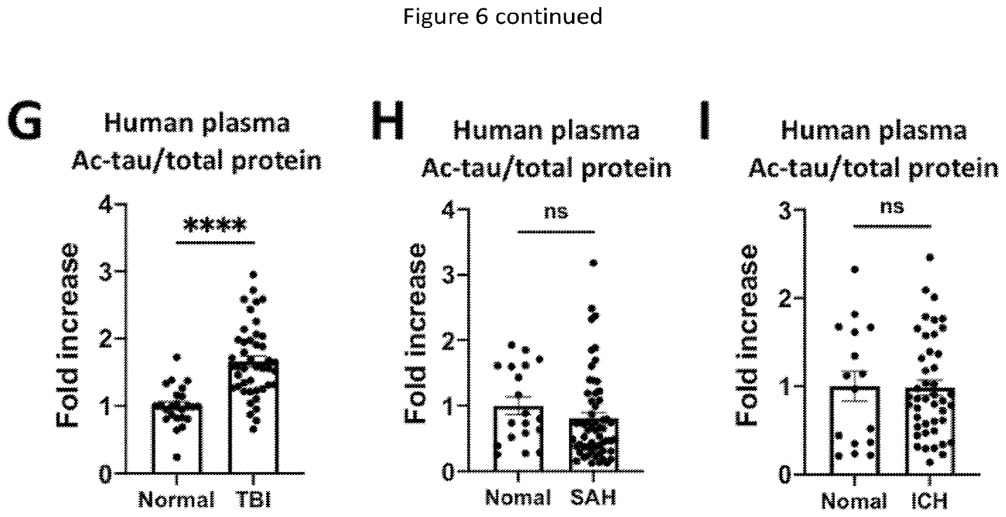

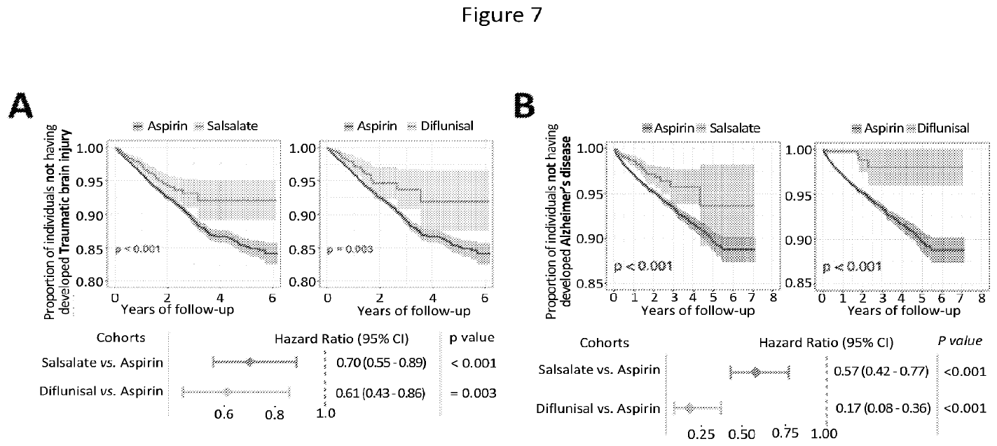

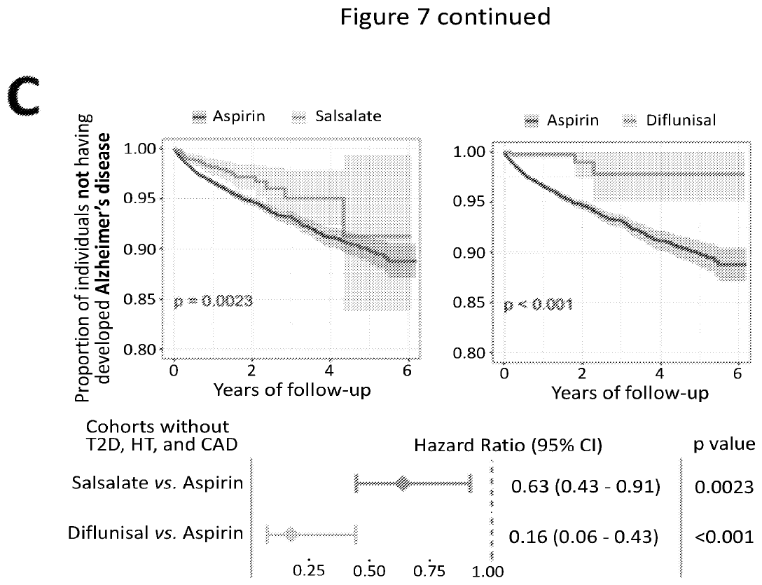

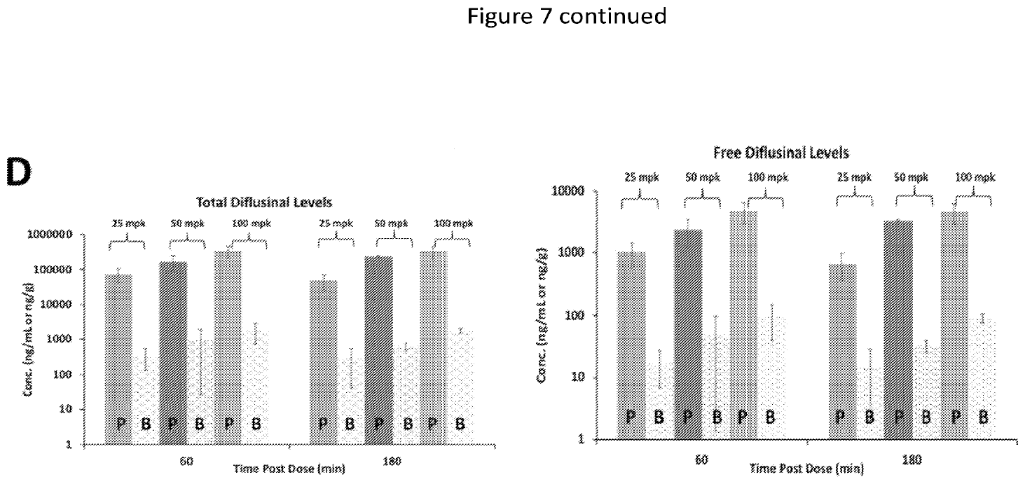

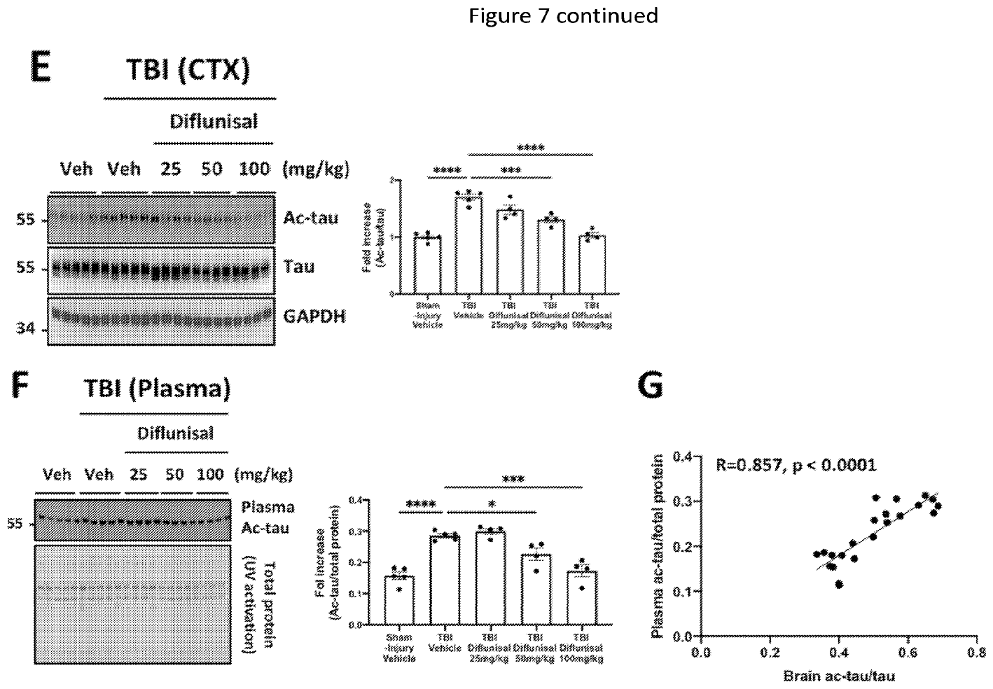

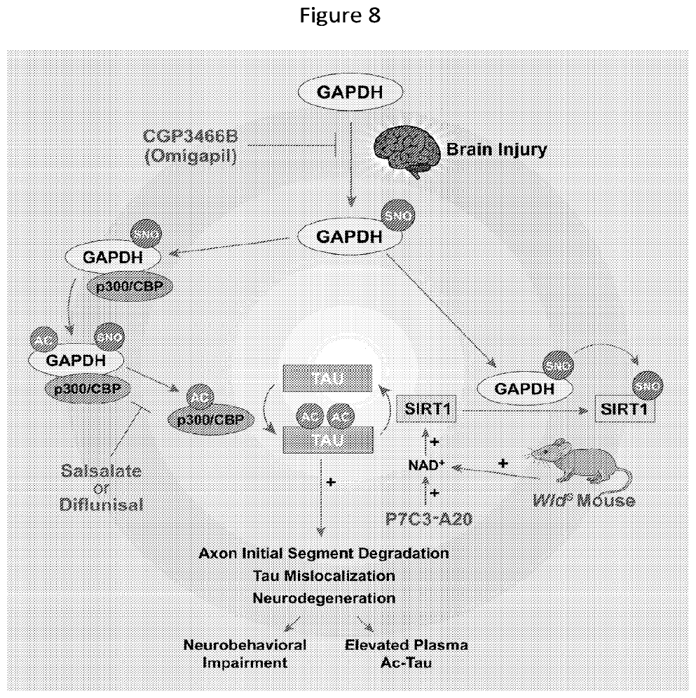

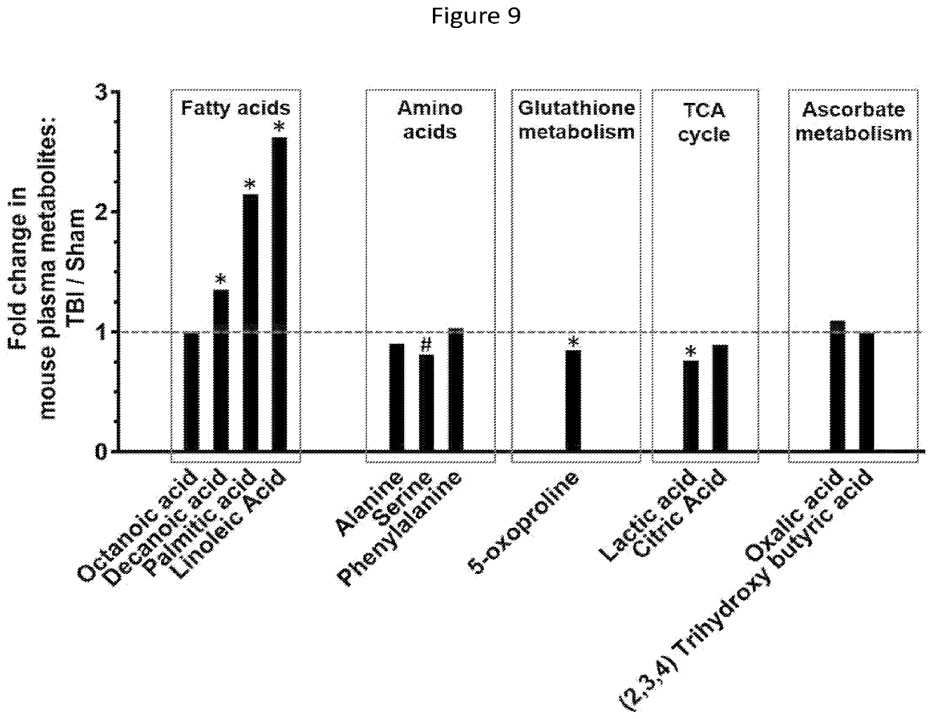

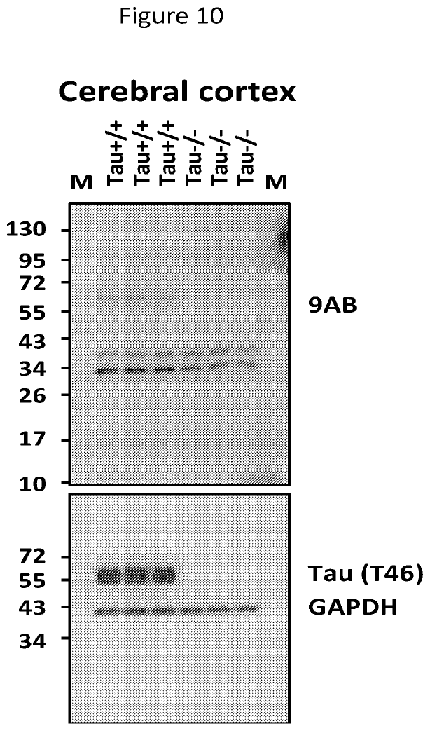

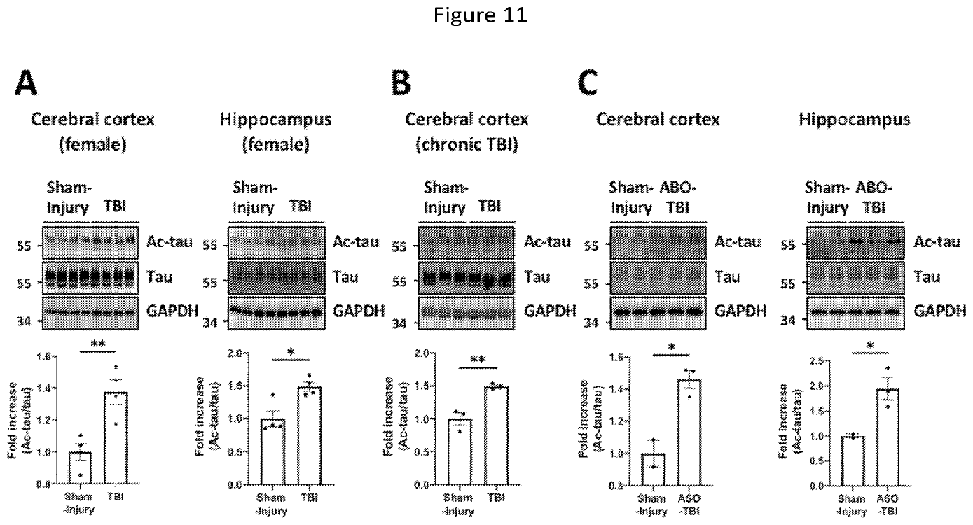

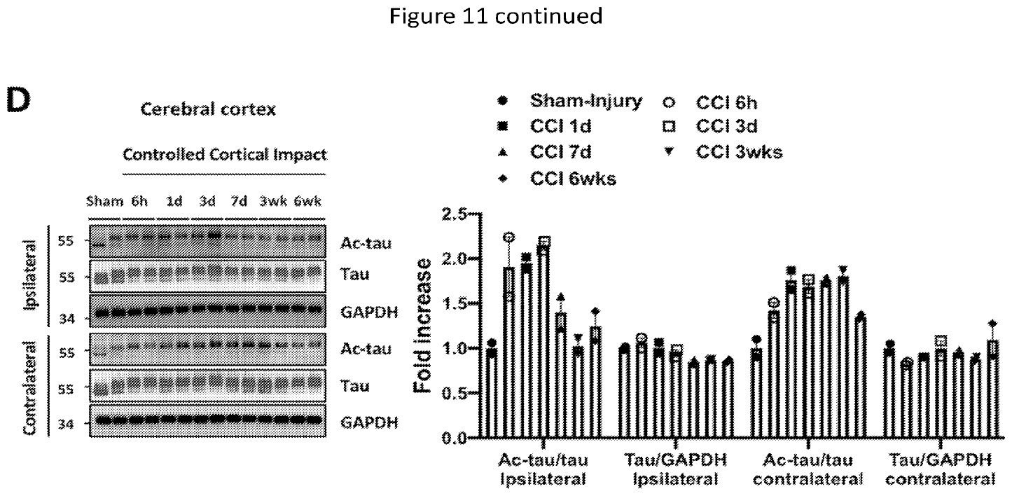

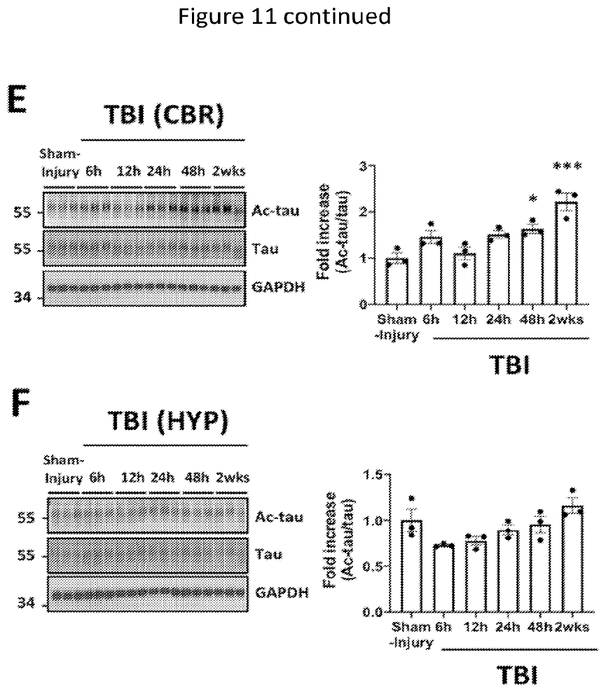

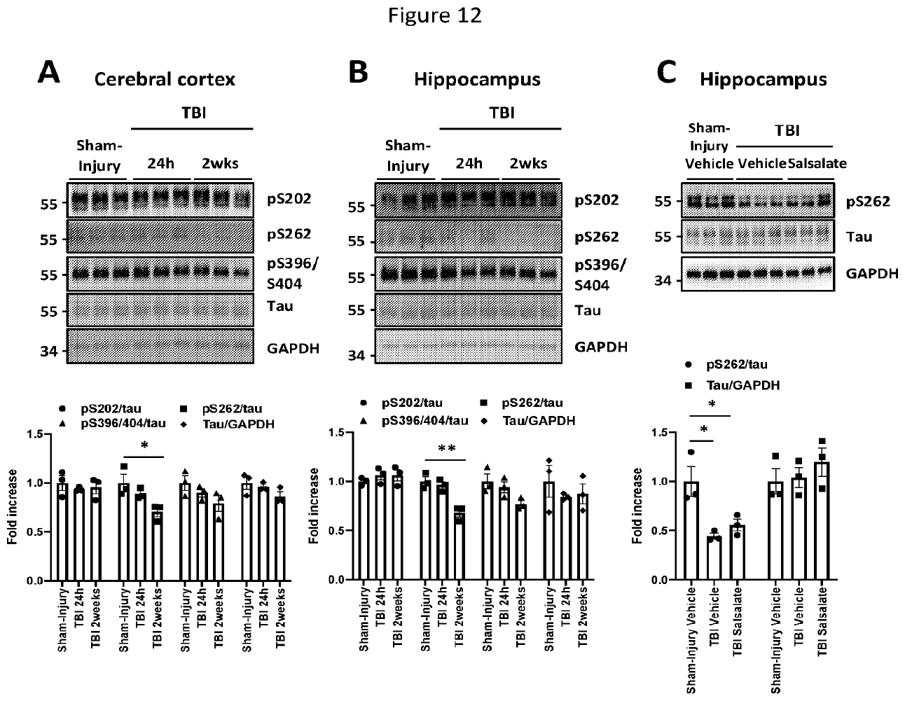

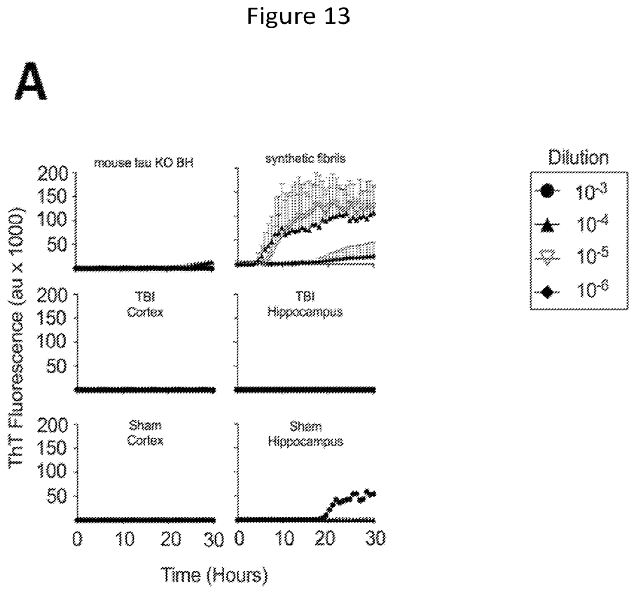

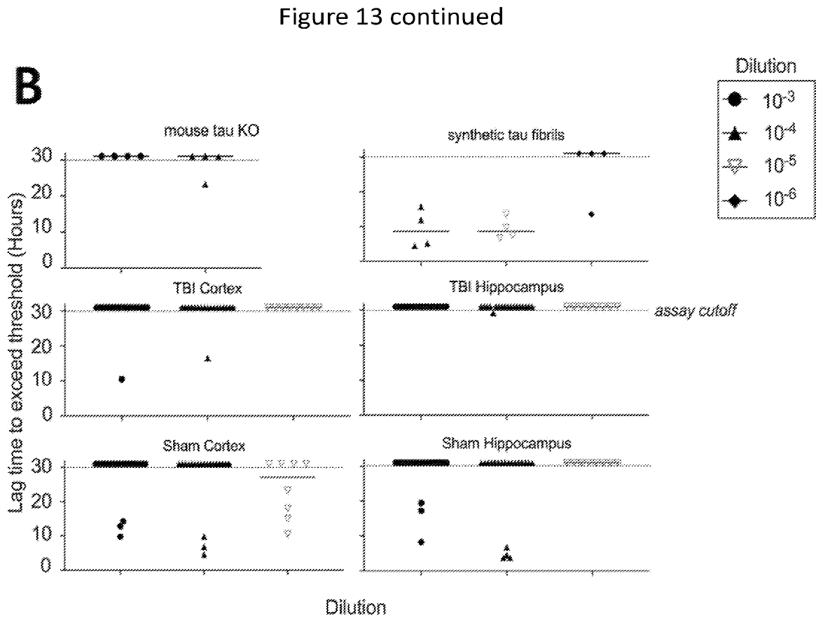

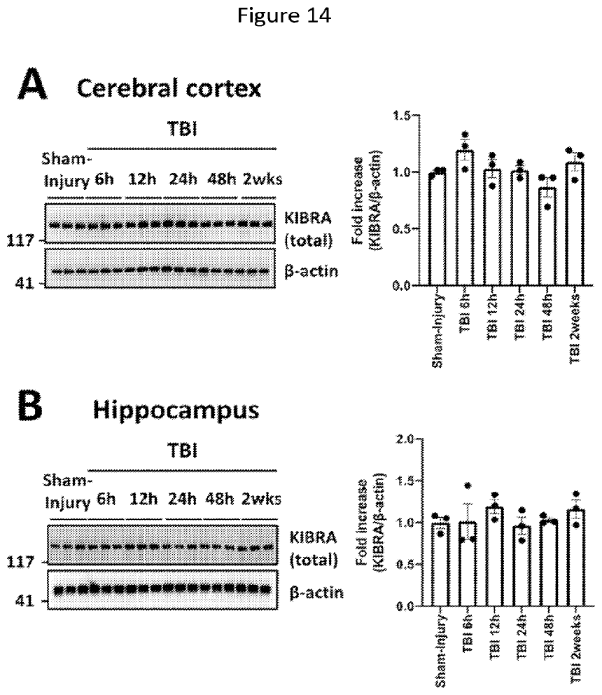

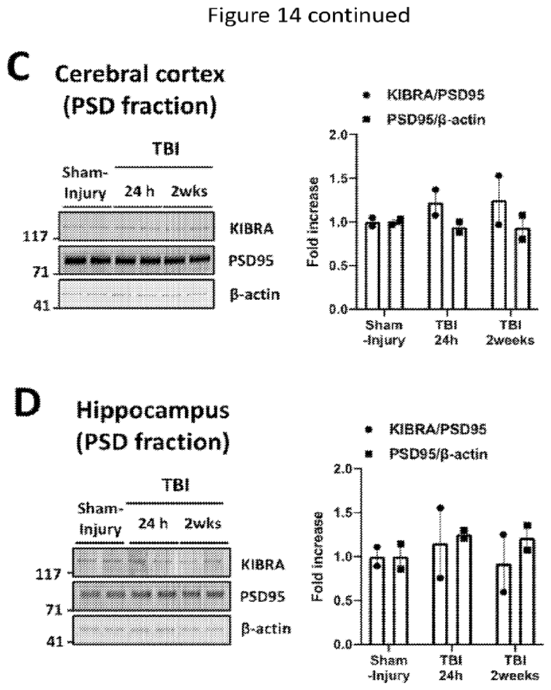

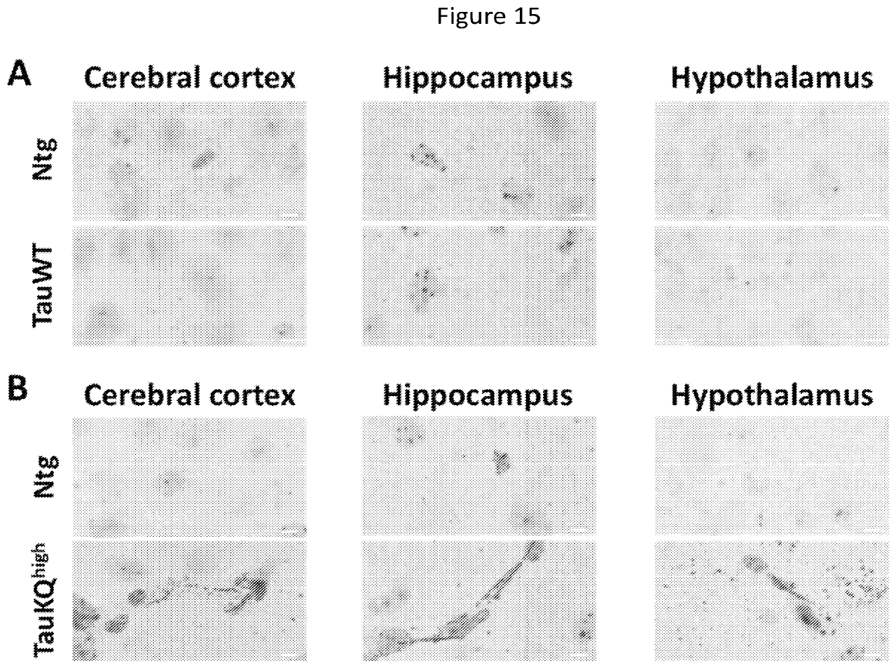

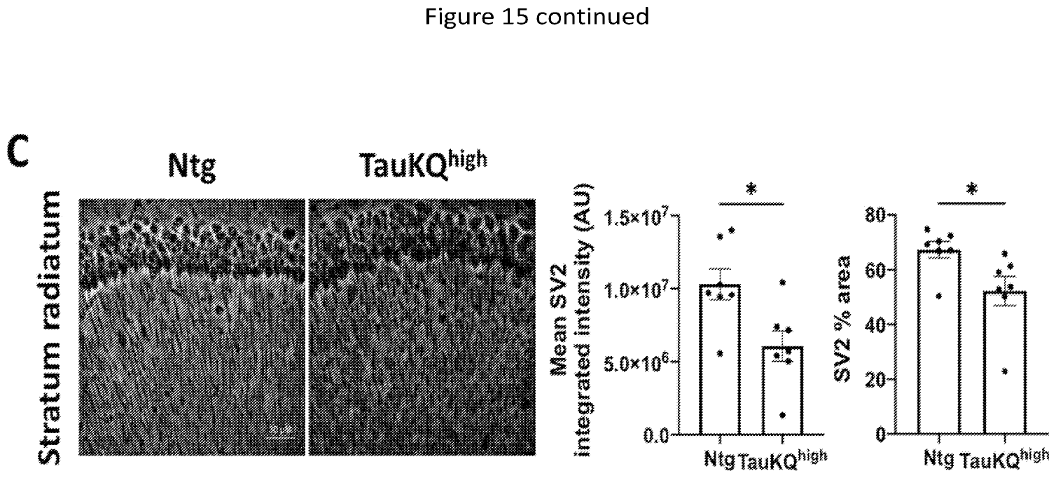

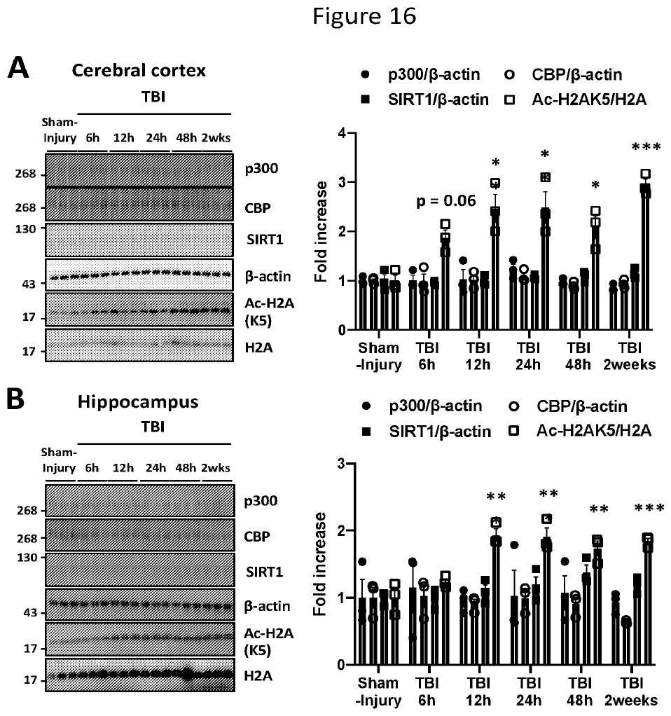

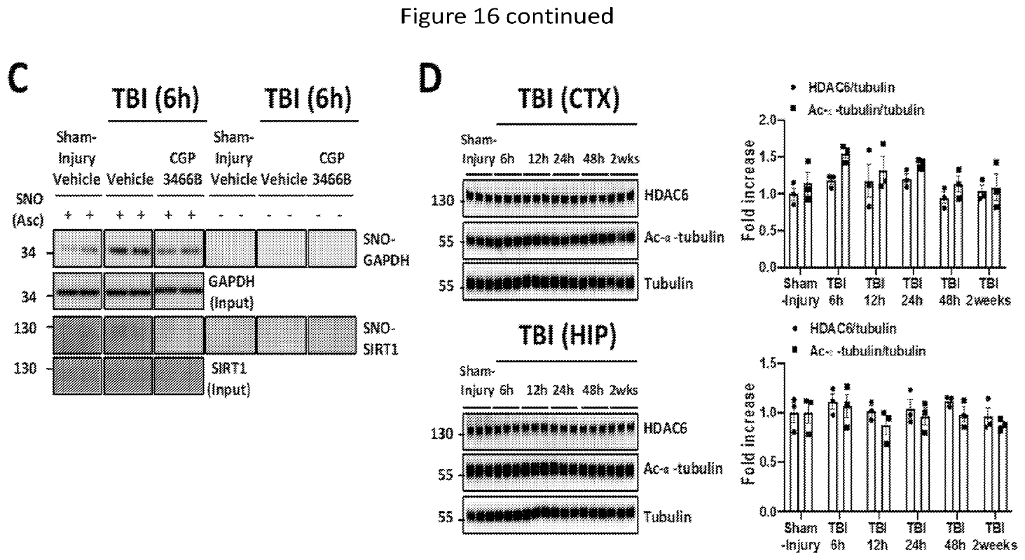

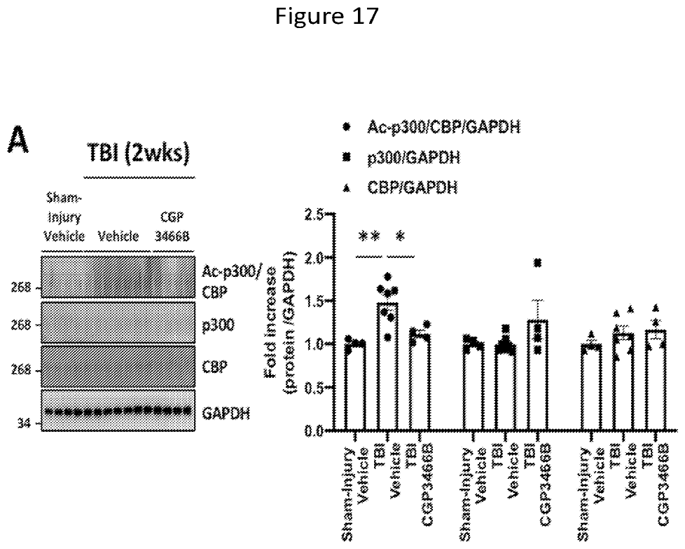

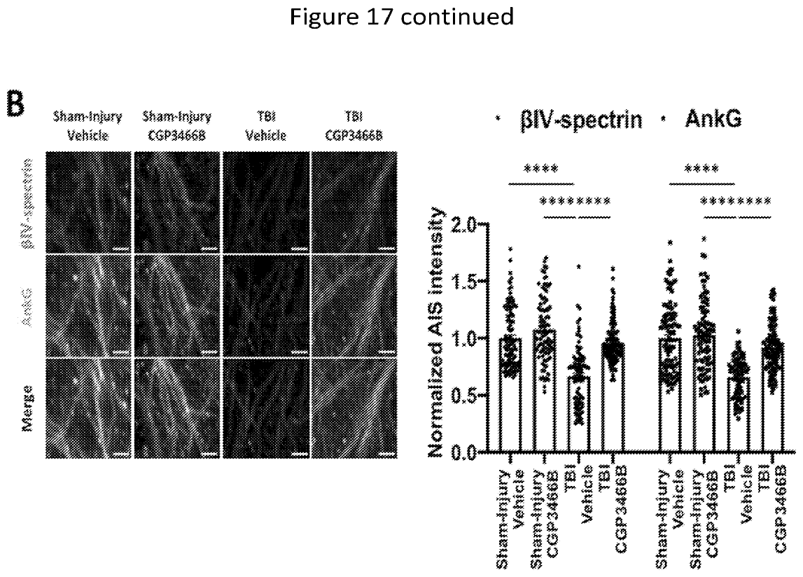

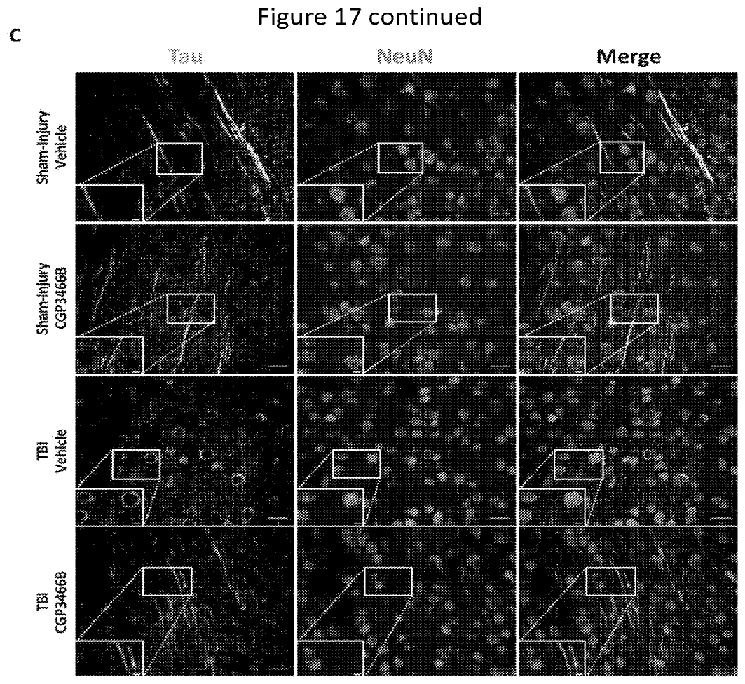

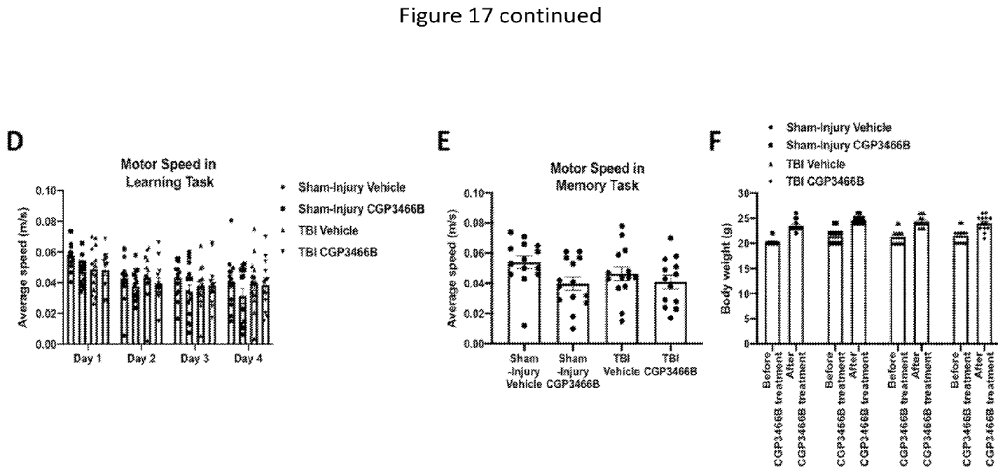

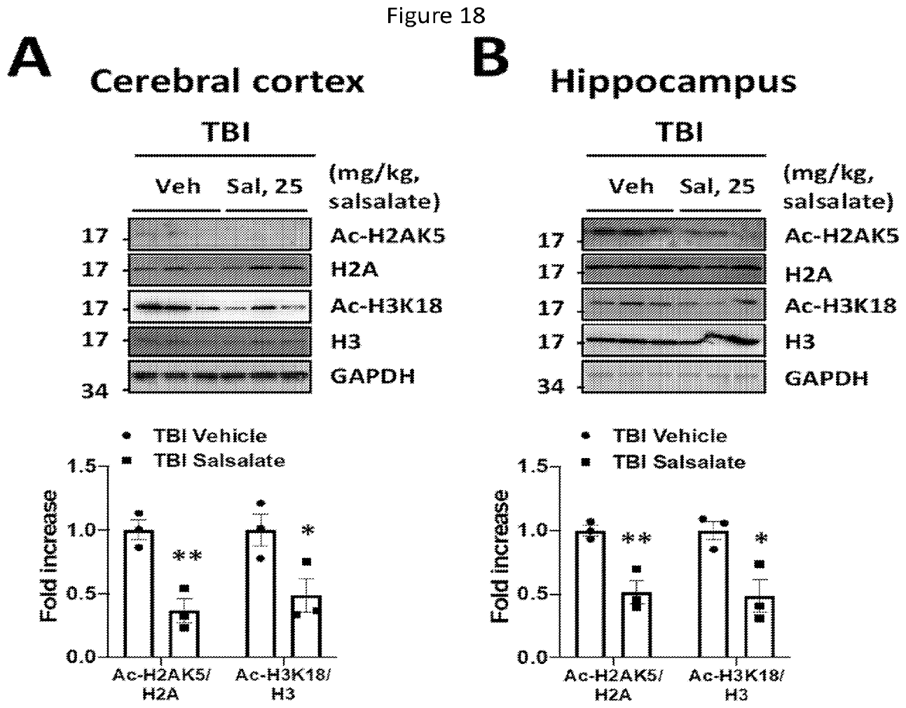

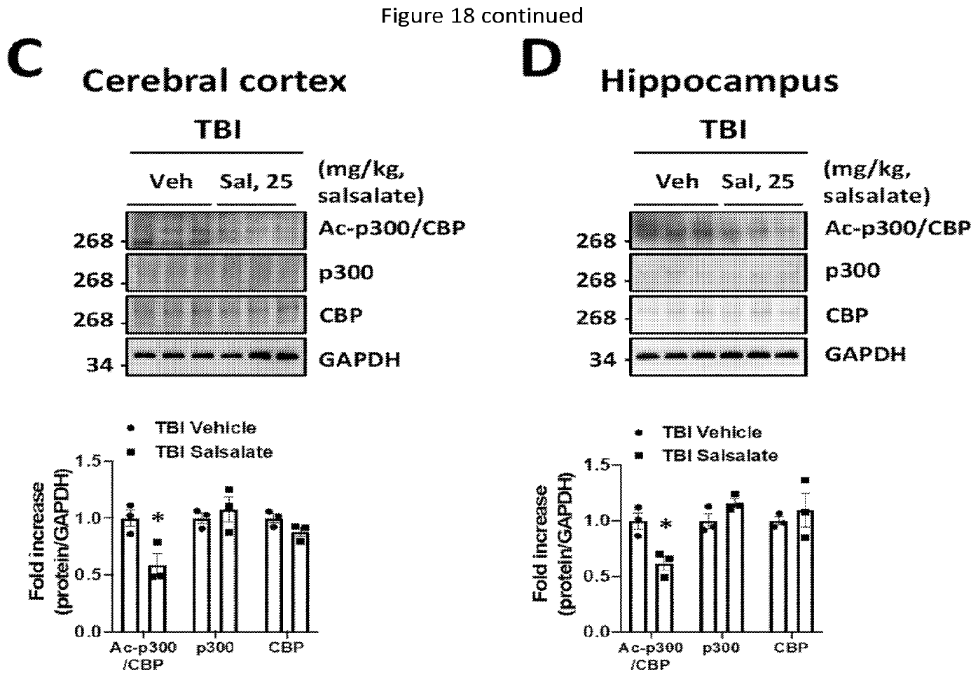

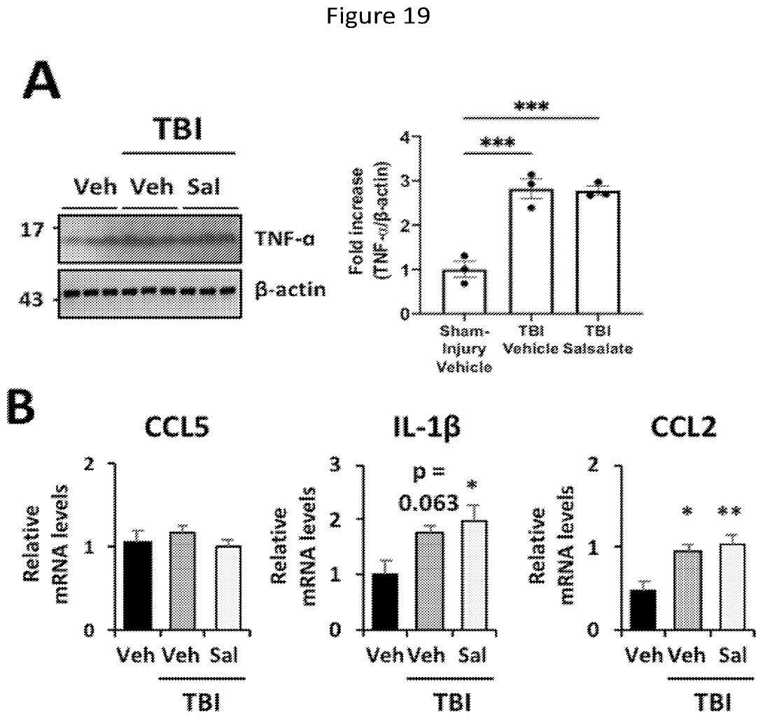

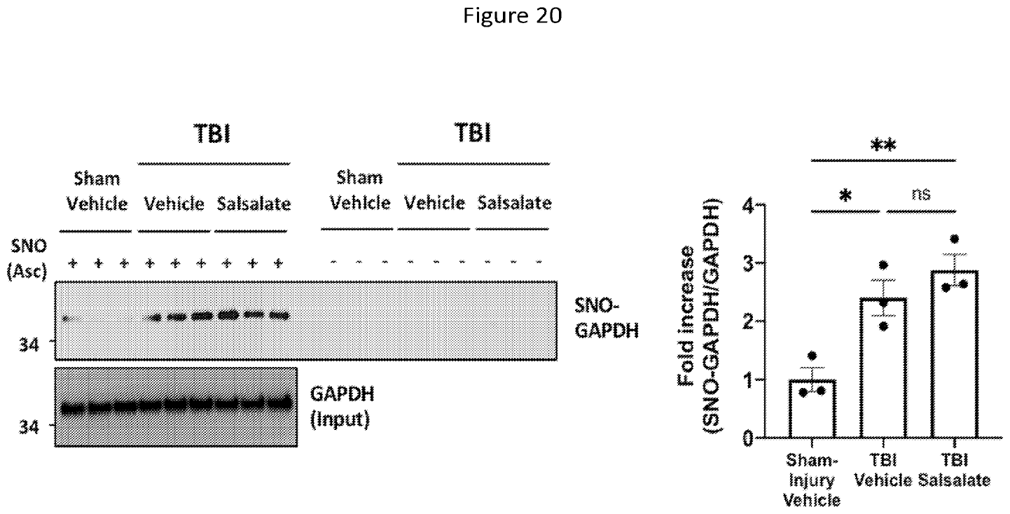

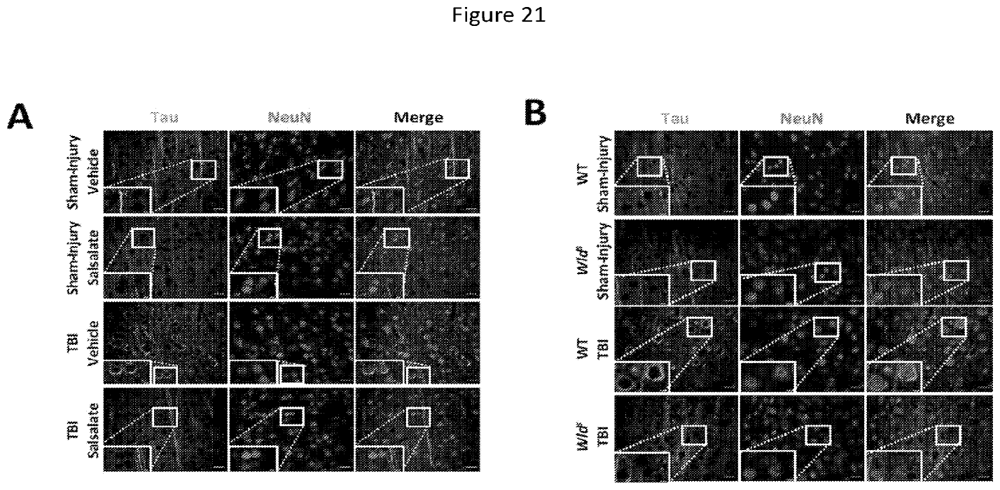

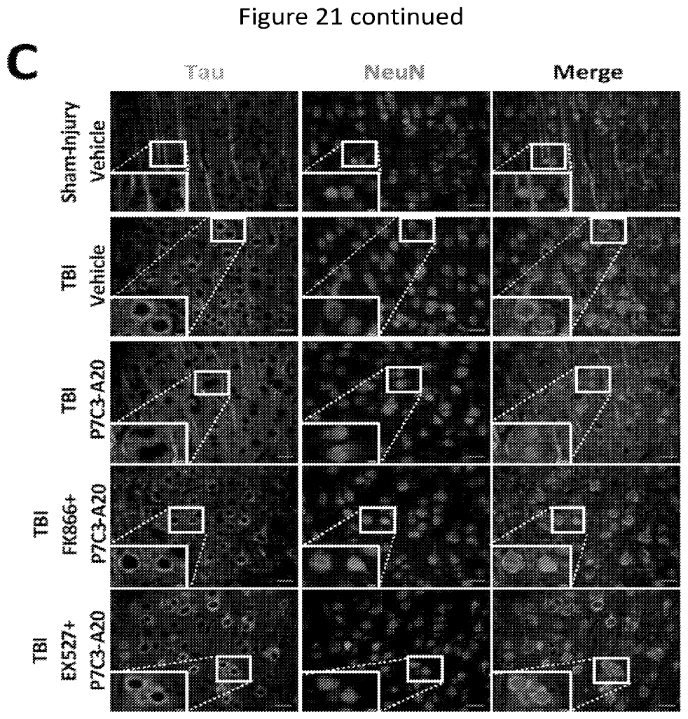

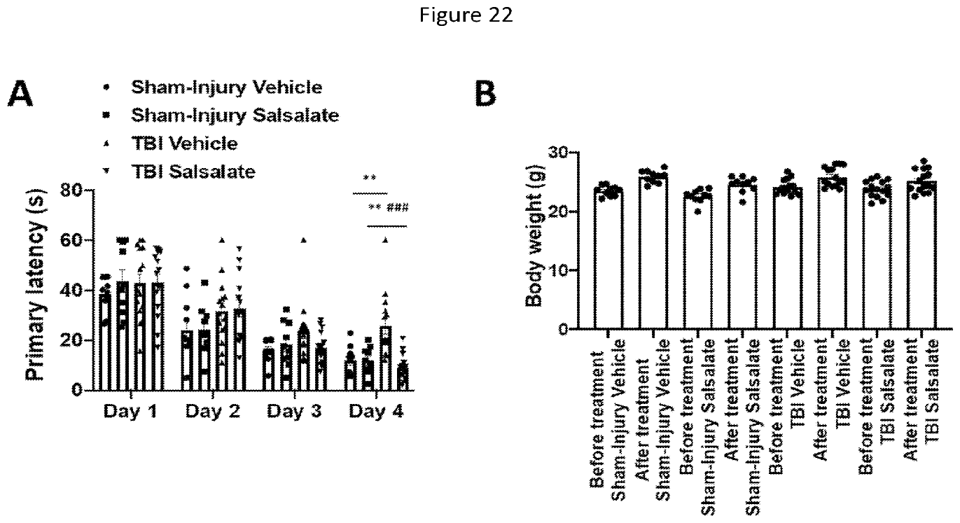

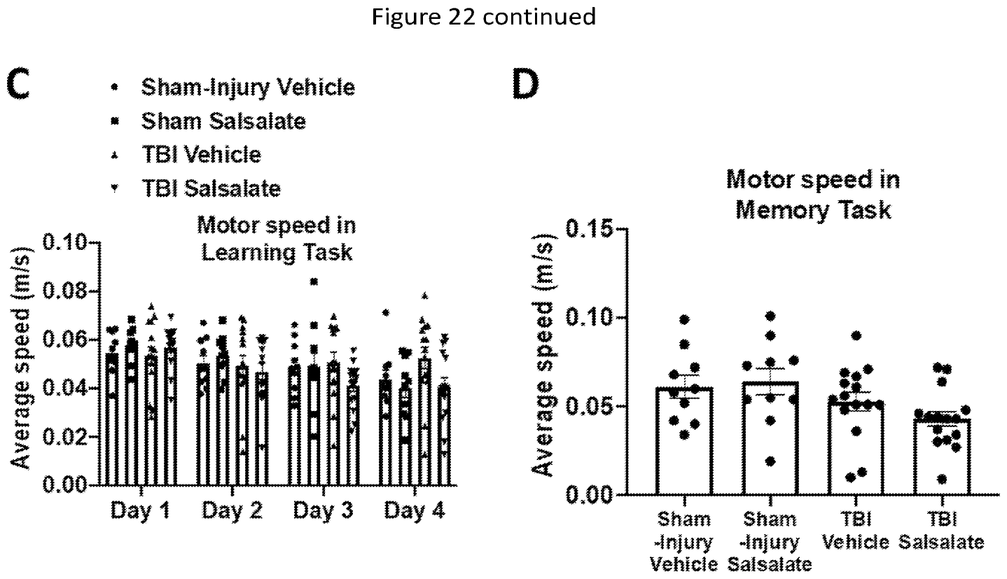

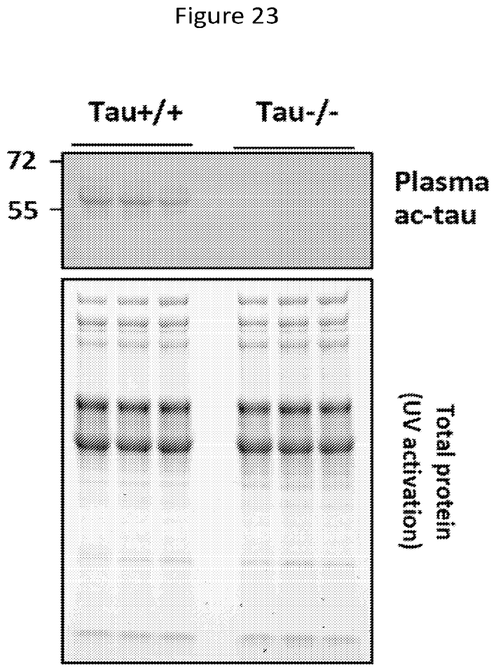

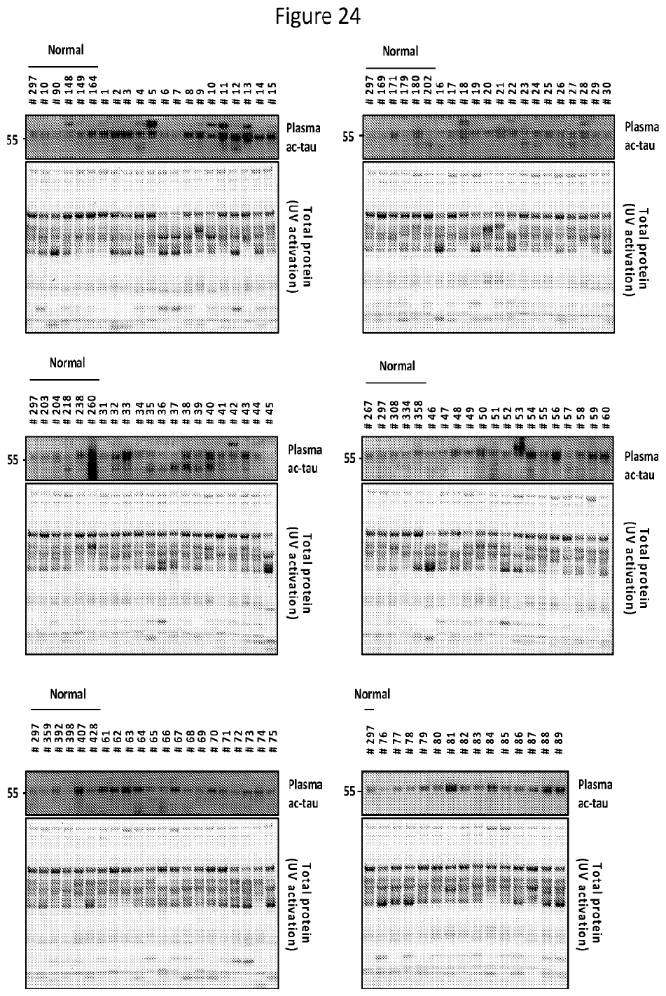

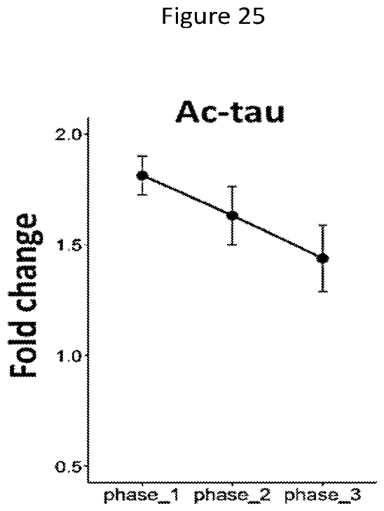

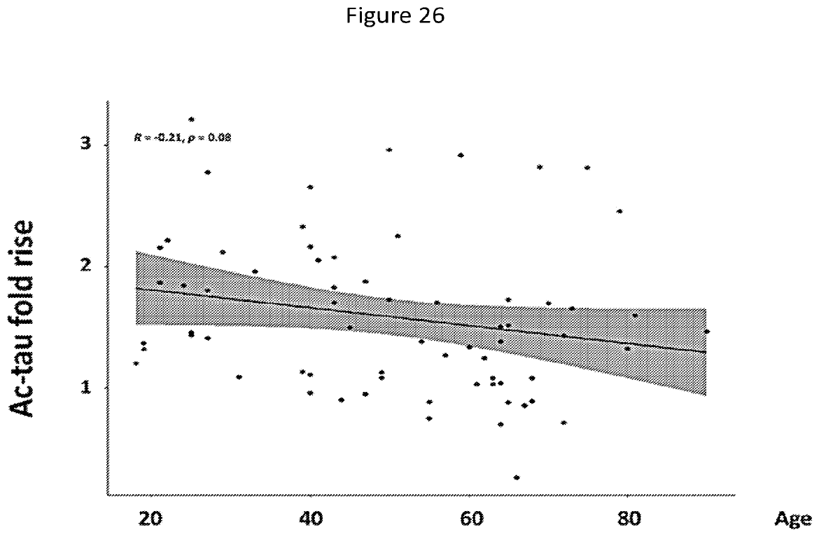

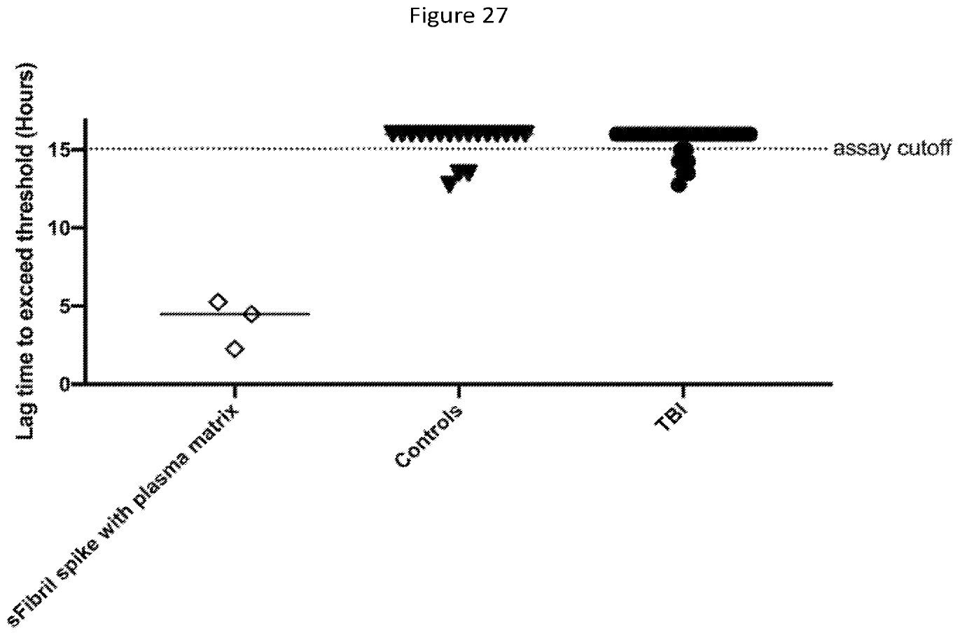

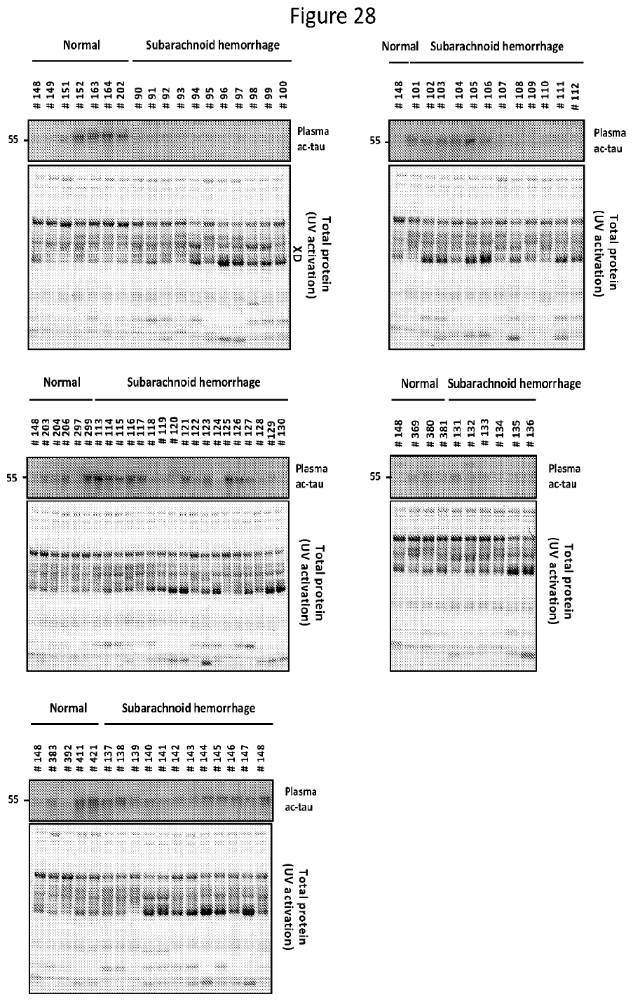

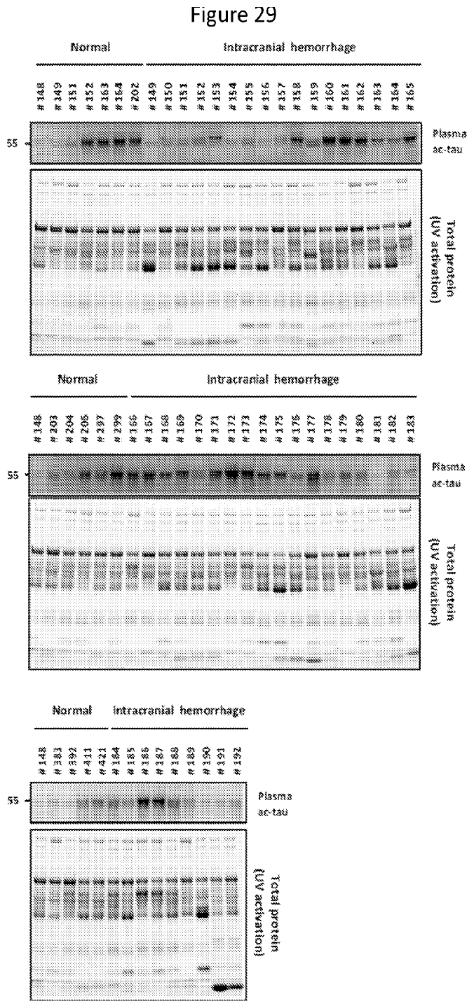

(FIG. 1A) Quantified western blot shows increased ac-tau 6 h-2 wks after TBI in cerebral cortex and hippocampus (Each group n=3, **p<0.01, ***p<0.001, ****p<0.0001 vs. Sham-Injury group, one-way ANOVA with Dunnett multiple comparisons test).

(FIG. 1B) Quantified western blot shows increased ac-tau in neurons (NeuN+, GFAP−), but not glia (NeuN−, GFAP+), of the cerebral cortex, with greater tau expression in neurons. (Each lane consists of pooled brain tissue from 3 animals, **p<0.01 vs. Sham-Injury group, Student's t-test).

(FIG. 1C) Quantified western blot shows TBI intensity-dependent increase in ac-tau. (Each group n=3-4, **p<0.01, ****p<0.0001 vs. Sham-Injury group, one-way ANOVA with Dunnett multiple comparisons test; PSI=pounds per square inch of explosive pressure).

(FIG. 1D) Quantified western blot shows reduced AIS proteins AnkG and βIV-spectrin after TBI, consistent with AIS degradation (Each group n=3, *p<0.05, **p<0.01, ***p<0.001 vs. Sham-Injury group, one-way ANOVA with Dunnett multiple comparisons test)

(FIG. 1E) Immunohistochemical staining for tau and the neuronal marker NeuN shows normal axonal localization of tau 2 weeks after sham-injury, and pathological tau mislocalization into the somatodendritic compartment 2 weeks after TBI (scale bar=5 μm). Images are representative of 3 animals per group.

(FIG. 1F) Blast-injury induces increased cleaved caspase-3 levels and LDH release in TauKQ (mimicking acetylated lysine) overexpressing cells compared to TauWT and TauKR (nonacetylatable tau mutant) transfected cells (***p<0.001, ****p<0.0001 vs. TauWT, TauKR transfected cells, one-way ANOVA and Tukey's post hoc analysis).

(FIG. 1G) TauKQ^{high} mice have axon degeneration in cerebral cortex, hippocampus and hypothalamus, which is absent from nontransgenic littermates (*p<0.05, Student's t-test).

FIGS. 2A-2G. SNO-GAPDH Mediates the Post-TBI p300/CBP Acetyltransferase Activation and Sirt1 Deacetylase Inhibition that Leads to Accumulated Ac-tau, AIS Degradation, Tau Mislocalization, Neurodegeneration, and Cognitive Deficits.

(FIG. 2A) Western blot and its quantification show significantly increased S-nitrosylation (SNO) of GAPDH and Sirt1 in cerebral cortex after TBI (n=3 per group, *p<0.05, **p<0.01, ***p<0.001 vs. Sham-Injury group, one-way ANOVA and Dunnett multiple comparisons test). “Ascorbate (Asc)—negative control” shows specificity of signal in the SNO-resin-assisted capture technique.

(FIG. 2B) Western blot and its quantification show that treatment of CGP3466B inhibits S-nitrosylation of GAPDH and Sirt1 at 0.014 mg/kg (Each group n-4, **p<0.01, ***p<0.001 vs. TBI+Vehicle group, one-way ANOVA and Tukey's post hoc analysis). Asc+ represents SNO, and Asc− represents control.

(FIG. 2C) Western blot and its quantification show that 0.014 mg/kg CGP3466B reduces ac-tau in cerebral cortex after TBI (Each group n=4-7, *p<0.05, ***p<0.001 vs. TBI+Vehicle group, one-way ANOVA and Tukey's post hoc analysis).

(FIG. 2D) CGP3466B protects mice from post-TBI AIS degradation in the cerebral cortex (scale bar=5 μm).

(FIG. 2E) CGP3466B protects mice from post-TBI tau mislocalization (scale bar=5 μm). Lower magnification pictures of the field from which these pictures were derived are shown in FIG. 16C.

(FIG. 2F) CGP3466B protects mice from post-TBI axon degeneration, as evidenced by silver staining of degenerating axons (scale bar=5 μm).

In (FIG. 2D)-(FIG. 2F), each group n=3-5, *p<0.05, **p<0.01, ****p<0.0001 vs. TBI+Vehicle group, one-way ANOVA and Tukey's post hoc analysis).

(FIG. 2G) CGP3466B protects mice from post-TBI impaired cognition in both learning and memory phases of the Barnes maze task (*p<0.05, **p<0.01, ***p<0.001, ****p<0.0001 vs. TBI+Vehicle group, repeated-measures two-way ANOVA (learning) and one-way ANOVA (memory) with Tukey's post hoc analysis).

FIGS. 3A-3E. Low-dose Salsalate-mediated Inhibition of p300/CBP Acetyltransferase Protects Mice from Post-TBI-induced Elevated Ac-tau, AIS Degradation, Tau Mislocalization, Neurodegeneration, and Cognitive Deficits.

(FIG. 3A) Low-dose salsalate dose-dependently reduces post-TBI elevations in ac-tau in the brain (n=3, *p<0.05, **p<0.01, ***p<0.001 vs. TBI+Vehicle group, one-way ANOVA and Tukey's post hoc analysis).

(FIG. 3B) Low-dose salsalate protects mice from post-TBI AIS degradation (scale bar=5 μm).

(FIG. 3C) Low-dose salsalate protects mice from post-TBI tau mislocalization (scale bar=5 μm).

(FIG. 3D) Low-dose salsalate protects mice from post-TBI axonal degeneration (scale bar=5 μm).

In (FIG. 3B)-(FIG. 3D), each group n=3, *p<0.05, **p<0.01, ***p<0.001, ****p<0.0001 vs. TBI+Vehicle group, one-way ANOVA and Tukey's post hoc analysis).

(FIG. 3E) Low-dose salsalate protects mice from post-TBI impairments in motor (foot slip assay) and cognitive (learning and memory in the Barnes maze) behavioral assays. (*p<0.05, **p<0.01, ***p<0.001, ****p<0.0001 vs. TBI+Vehicle group, repeated-measures two-way ANOVA (learning) and one-way ANOVA (memory) with Tukey's post hoc analysis.

FIGS. 4A-4C. Wld^{S} Mice are Protected from Post-TBI-induced Elevated Ac-tau, AIS Degradation, and Tau Mislocalization.

(FIG. 4A) Wld^{S} mice are resistant to post-TBI elevations in ac-tau in the brain (for each group n=4, *p<0.05, **p<0.01 vs. WT+TBI group, one-way ANOVA and Tukey's post hoc analysis).

(FIG. 4B) Wld^{S} mice are resistant to post-TBI AIS degradation (scale bar=5 μm).

(FIG. 4C) Wld^{S} mice are resistant to post-TBI tau mislocalization (scale bar=5 μm).

In (FIG. 4B)-(FIG. 4C), each group n=3, ****p<0.0001 vs. WT+TBI group, one-way ANOVA and Tukey's post hoc analysis).

FIGS. 5A-5E. P7C3-A20 Treatment Protects Mice from Post-TBI-induced Elevated Ac-tau, AIS Degradation, and Tau Mislocalization.

(FIG. 5A) P7C3-A20 treatment rescued normal NAD⁺ levels after TBI, which was blocked by co-administration of FK866 (each group n=3, **p<0.01, ***p<0.001, one-way ANOVA and Tukey's post hoc analysis).

(FIG. 5B) P7C3-A20 treatment protects mice from post-TBI elevations in ac-tau in the brain (each group n=3-4, *p<0.05, **p<0.01, ***p<0.001, ****p<0.0001, one-way ANOVA and Tukey's post hoc analysis). This protective effect is blocked by treatment with the NAMPT inhibitor FK866 or the Sirt1 Inhibitor EX527.

(FIG. 5C) P7C3-A20 treatment protects mice from post-TBI AIS degradation (n=3 per group, ****p<0.0001, one-way ANOVA and Tukey's post hoc analysis). This protective effect is blocked by treatment with the NAMPT inhibitor FK866 or the Sirt1 Inhibitor EX527.

(FIG. 5D) P7C3-A20 treatment protects mice from post-TBI tau mislocalization (each group n=3, ****p<0.0001, one-way ANOVA and Tukey's post hoc analysis, scale bar=5 μm). This protective effect is blocked by treatment with the NAMPT inhibitor FK866 or the Sirt1 Inhibitor EX527.

(FIG. 5E) SNO-GAPDH is not affected by P7C3-A20 (western blot, each group n=3, *p<0.05, **p<0.01, one-way ANOVA and Tukey's post hoc analysis). “Ascorbate (Asc)—negative control” shows specificity of signal in the SNO-resin-assisted capture technique.

FIGS. 6A-6I. Elevated blood plasma ac-tau is a biomarker of TBI in mice and humans.

(FIG. 6A) Western blot shows that TBI increases plasma ac-tau. As tau and IgG have closely similar molecular weights, secondary antibody alone served as a control to ensure there was no cross reactivity with any residual IgG after IgG depletion.

(FIG. 6B) Western blot and its quantification show that CGP3466B significantly reduced plasma ac-tau levels after TBI (Each group n=4-5 with each lane representing a separated animal, **p<0.01, ***p<0.001 vs. TBI+Vehicle group, one-way ANOVA and Tukey's post hoc analysis).

(FIG. 6C) Western blot and its quantification show that salsalate reduces plasma ac-tau levels (Each group n=5-6 with each lane representing a separated animal, *p<0.05, ***<0.001 vs. TBI+Vehicle group, one-way ANOVA and Tukey's post hoc analysis).

(FIG. 6D) A repeat experiment in an independent cohort of animals confirmed the results shown in panel C. Each lane represents a separate animal. For both (FIG. 6C) and (FIG. 6D), (**p<0.01, TBI+Vehicle group, one-way ANOVA and Tukey's post hoc analysis).

(FIG. 6E) Western blot and its quantification show that P7C3-A20 significantly reduced plasma ac-tau levels after TBI (Each group n=5 with each lane representing a separated animal, ***p<0.001 vs. TBI+Vehicle group, one-way ANOVA and Tukey's post hoc analysis).

(FIG. 6F) Plasma Nfl, UCHL1 and GFAP, but not pTau181/Tau, levels are higher in TBI cohorts at 24 hours after injury, compared to controls (*p<0.05, **p<0.01, ***p<0.001)

(FIG. 6G) The mean level of ac-tau was significantly higher in the TBI cohort at 24 hours in comparison to the controls (1.8±0.58 vs 1.16±0.5, ****p<0.0001).

(FIG. 6H) The mean level of ac-tau in the subarachnoid hemorrhage (SAH) cohort at 24 hours was no different from controls.

(FIG. 6I) The mean level of ac-tau in the intracranial hemorrhage (ICH) cohort at 24 hours was no different from controls.

FIGS. 7A-7G. Diflunisal usage is associated with decreased incidence of TBI and AD in people, and with inhibition of ac-tau after TBI in mice.

(FIG. 7A) Longitudinal analyses reveal that salsalate and diflunisal usages reduce risk of traumatic brain injury (TBI) in all patient data from the IBM® MarketScan® Medicare Supplemental Database. The un-stratified Kaplan-Meier curves, conducted propensity score stratified (n strata=10) log-rank test and Cox model, and Hazard ratio and 95% confidence interval for two cohort studies, were illustrated for both A and B. Two cohort studies were conducted including: (i) salsalate users and aspirin users, and (ii) diflunisal users and aspirin users. Using propensity score stratified survival analyses by adjusting the initiation time of drugs, enrollment history, age and gender, and disease comorbidities (diabetes, or hypertension, or coronary artery disease). Propensity score stratified Cox-proportional hazards models were used to conduct statistical inference for the hazard ratios.

(FIG. 7B) Longitudinal analyses reveal that salsalate and diflunisal usage in the same group as (A) is also associated with reduced incidence of AD in people.

(FIG. 7C) Subgroup analyses after excluding patients with type 2 diabetes, hypertension, or coronary artery disease (known risk factors for AD) further confirms that salsalate or diflunisal usage is associated with decreased incidence of AD.

(FIG. 7D) LC-MS/MS analysis shows modest penetration of diflunisal into mouse brain but the plasma:brain ratio is decreased when protein binding is taken into account and free drug levels are compared. Drug levels in plasma and brain were determined by LC-MS/MS analysis after mice were administered three different concentrations of diflunisal and euthanized 60 or 180 min later, followed by collection of blood and perfusion with saline, prior to harvesting brain tissue. Rapid equilibrium dialysis was used to determine binding of diflunisal in mouse plasma and brain homogenate. ‘P’ and ‘B’ denote plasma and brain, respectively.

(FIG. 7E) Diflunisal treatment dose-dependently reduces post-TBI elevations in ac-tau in the brain (Each group n=4-5, ***p<0.001, ****p<0.0001 vs. TBI+Vehicle group, one-way ANOVA and Tukey's post hoc analysis).

(FIG. 7F) Western blot and its quantification show that diflunisal dose-dependently reduced plasma ac-tau levels after TBI (Each group n=4-5 with each lane representing a separate animal, *p<0.05, ***p<0.001, ****p<0.0001 vs. TBI+Vehicle group, one-way ANOVA and Tukey's post hoc analysis).

(FIG. 7G) There is a significant correlation between brain and plasma ac-tau levels after brain injury (data from panel C and D; R=0.857, p<0.0001).

FIG. 8. Reducing brain injury-induced neuronal tau acetylation is neuroprotective.

FIG. 9. Nonbiased whole-metabolome analysis of mouse plasma 1 week after TBI or sham injury of 8-week old C57BL/6J mice shows alterations in plasma levels of metabolites in fatty acid (decanoic acid, palmitic acid, linoleic acid), amino acid (serine), glutathione (5-oxoproline), and TCA cycle (lactic acid) pathways consistent with what has been previously reported in human TBI plasma. (n=10 per group, *p<0.05, #trending).

FIG. 10. Specificity of antibody for mouse ac-tau. 9AB antibody generated by the Gan laboratory against mouse tau acetylated at K263 and K270 recognizes ac-tau in brain extract from wild type mice, but not from tau knockout mice. The presence or absence of tau in wild type or tau knockout mice, respectively, was confirmed by western blot for tau with T46 antibody (Invitrogen). Western blot for GAPDH shows equal loading of protein across lanes. (M=molecular weight marker lanes)

FIGS. 11A-11F. TBI increase ac-tau in both sexes, across species, and in different forms of TBI.

(FIG. 11A) Western blot and its quantification show that a single TBI also increases ac-tau in cerebral cortex and hippocampus of female mice 2 weeks later. (Each group n=4, *p<0.05, **p<0.01 vs. Sham-Injury group, Student's t-test).

(FIG. 11B) Western blot and its quantification show that ac-tau is significantly increased 17 months after a single TBI in male C57BL/6J mice (Each group n=3, *p<0.05 vs. Sham-Injury group, Student's t-test).

(FIG. 11C) Western blot and its quantification show that cortical and hippocampal ac-tau are significantly increased 7 months after acoustic blast overpressure-injury in male Long-Evans rats (Each group n=2-3, *p<0.05 vs. Sham-Injury group, Student's t-test).

(FIG. 11D) Western blot and its quantification show that controlled cortical impact injury of male wild type mice increased ac-tau in the ipsilateral (6 h, 1 d, 3 d after injury) and contralateral side (1 d, 3 d, 7 d, 3 wks), with no change in total tau levels (Each group n=2).

(FIG. 11E) Western blot and its quantification show increased ac-tau 48 h-2 weeks after TBI in cerebellum (Each group n=3, *p<0.05, ***p<0.001 vs. Sham-Injury group, one-way ANOVA with Dunnett multiple comparisons test).

(FIG. 11F) Western blot and its quantification show no increase in hypothalamic ac-tau after TBI (Each group n=3).

FIGS. 12A-12C. TBI does not acutely increase tau phosphorylation or alter total levels of tau.

(FIG. 12A), (FIG. 12B) Western blot and its quantification show no increase in tau phosphorylation at residues S202, S262, S396, and S404, either 24 hours or 2 weeks after TBI. Phosphorylation of tau at S262 is significantly decreased in cortex and hippocampus 2 weeks after TBI (Each group n=3, *p<0.05, **p<0.01 vs. Sham-Injury group, one-way ANOVA with Dunnett multiple comparisons test).

(FIG. 12C) Western blot and its quantification show that phosphorylation of tau at S262 remains significantly decreased in hippocampus even when acetylation of tau is blocked by treating mice after TBI with the p300/CBP inhibitor salsalate (Each group n=3, *p<0.05 vs. Sham-Injury group, one-way ANOVA and Tukey's post hoc analysis).

In (FIG. 12A), (FIG. 12B), and (FIG. 12C), total tau levels remained constant at all times after TBI.

FIGS. 13A-13B. Tau acetylation 2 weeks after TBI does not impact tau seeding capacity.

(FIG. 13A) TBI and sham-injury brain homogenates were used to seed the Alzheimer's disease real-time quaking-induced conversion (AD RT-QuIC) assay. Each curve represents the thioflavin T (ThT) fluorescence readouts of the mean±SD of quadruplicate wells seeded at the dilutions indicated.

(FIG. 13B) Lag time to exceed the ThT fluorescence threshold is shown (determined as 100×SD of the baseline), with each data point representing a single well run in quadruplicate for 5 biological replicates of TBI hippocampus, TBI cerebral cortex, sham-injury hippocampus, and sham-injury cerebral cortex. Tissue dilutions are indicated, with quadruplicate wells of 5 biological replicates evaluated at 10⁻³, 10⁻⁴, and 2 biological replicates at 10⁻⁵. Mouse tau KO brain homogenate was evaluated as a negative control, and synthetic fibrils generated from recombinant tau served as a positive control.

FIGS. 14A-14D. Kidney/BRAin (KIBRA) expression is unchanged after TBI.

(FIG. 14A), (FIG. 14B) Western blot and its quantification show that total KIBRA expression remained constant across all time points after TBI (Each group n=3).

(FIG. 14C), (FIG. 14D) Western blot and its quantification show that postsynaptic KIBRA expression, defined as post-synaptic density 95 protein fraction (PSD95), is not altered 24 hours or 2 weeks after TBI (Two pooled samples in each lane).

FIGS. 15A-15C. TauKQ^{high} animals display elevated axon degeneration and reduced synapses.

(FIG. 15A) Representative images show lack of axonal degeneration in cerebral cortex, hippocampus and hypothalamus of transgenic mice overexpressing wild type human tau (scale bar=5 μm), as expected.

(FIG. 15B) Representative images show prominent axonal degeneration in cerebral cortex, hippocampus and hypothalamus of transgenic mice with human tau mutated to mimic acetylation of K274 and K281 (KQ) (TauKQ^{high}), relative to nontransgenic littermates (scale bar=5 μm).

(FIG. 15C) TauKQ^{high} animals have reduced synaptic vesicle protein 2 (SV2) levels in stratum radiatum of hippocampus relative to nontransgenic littermates. (*p<0.05, Student's t-test, scale bar=30 μm).

FIGS. 16A-16D. TBI regulates p300/CBP and Sirt1 activity, but not HDAC6.

(FIG. 16A) Western blot and its quantification show that TBI significantly increased acetylation of histone 2A lysine 5, a well-established substrate of p300/CBP and Sirt1, in the cerebral cortex in a time-dependent manner, without affecting expression levels of p300, CBP, or Sirt1.

(FIG. 16B) Western blot and its quantification show that TBI significantly increased acetylation of histone 2A lysine 5, a well-established substrate of p300/CBP and Sirt1, in the hippocampus in a time-dependent manner, without affecting expression levels of p300, CBP, or Sirt1.

For (FIG. 16A) and (FIG. 16B), each group n=3, *p<0.05, **p<0.01, ***p<0.001 vs. Sham-Injury group, one-way ANOVA with Dunnett multiple comparisons test.

(FIG. 16C) Treatment of CGP3466B inhibits S-nitrosylation of GAPDH and Sirt1 at 0.014 mg/kg (Each group n=2, separation between squares represents cut in gel). Asc+ represents SNO, and Asc− represents control.

(FIG. 16D) Western blot and its quantification show that TBI does not affect expression of HDAC6, or its activity as measured by acetylated α-tubulin in the cerebral cortex and hippocampus.

FIGS. 17A-17F. CGP3466B treatment initiated 24 hours after TBI blocks tau mislocalization and does not affect speed during behavioral testing or body weight.

(FIG. 17A) Western blot and its quantification show that 0.014 mg/kg CGP3466B reduces ac-p300/CBP in cerebral cortex after TBI (Each group n=4-7, *p<0.05, **p<0.01 vs. TBI+Vehicle group, one-way ANOVA and Tukey's post hoc analysis).

(FIG. 17B) CGP3466B protects mice from post-TBI AIS degradation in the hippocampus (Each group n=3, ****p<TBI+Vehicle group, one-way ANOVA and Tukey's post hoc analysis, scale bar=5 μm).

(FIG. 17C) Images shown are lower power fields of tau localization in neurons from which higher power figures shown in FIG. 2E were derived, with the higher power field shown in white box. (scale bar=20 μm for original image, 5 μm for inset).

(FIG. 17D) CGP3466B treatment did not affect average speed during the learning phase of the Barnes maze task.

(FIG. 17E) CGP3466B treatment did not affect average speed during the memory phase of the Barnes maze task.

(FIG. 17F) CGP3466B treatment did not affect body weight.

FIGS. 18A-18D. Low-dose salsalate inhibits p300/CBP activity.

(FIG. 18A), (FIG. 18B) Inhibition of p300/CBP by salsalate was assessed by measurement of acetylation of well-known substrates, H2AK5 and H3K18. Salsalate treatment significantly reduced levels of ac-H2AK5 and ac-H3K18 in cerebral cortex and hippocampus (Each group n=3, *p<0.05, **p<0.01 vs. TBI+Vehicle group, Student's t-test).

(FIG. 18C), (FIG. 18D) Western blot and its quantification show that acetylation of p300/CBP was also significantly reduced by treatment with salsalate (Each group n=3, *p<0.05 vs. TBI+Vehicle group, Student's t-test).

FIGS. 19A-19B. Low-dose salsalate is devoid of anti-neuroinflammatory effect after TBI.

(FIG. 19A) Western blot analysis and its quantification show that the proinflammatory cytokine TNF-alpha is upregulated in the cerebral cortex in response to TBI injury, and that this increase is not affected by treatment with 25 mg/kg of salsalate. (Each group n=3, ***p<0.001 vs. Sham-Injury+Vehicle group, one-way ANOVA and Tukey's post hoc analysis).

(FIG. 19B) qPCR analysis shows that IL-1β and CCL2 are upregulated in TBI-injured cerebral cortex, and that this increase is not attenuated by treatment with 25 mg/kg of salsalate. Levels of CCL5 were not affected by either TBI or salsalate. (Each group n=3, *p<0.05, **p<0.01 vs. Sham-Injury+Vehicle group, one-way ANOVA and Tukey's post hoc analysis).

FIG. 20. Low-dose salsalate does not affect S-nitrosylation of GAPDH after TBI. Western blot and its quantification show that S-nitrosylation of GAPDH is not affected by treatment with 25 mg/kg salsalate. (Each group n=3, *p<0.05, **p<0.01 vs. Sham-Injury+Vehicle group, one-way ANOVA and Tukey's post hoc analysis). “Ascorbate (Asc)—negative control” shows specificity of signal in the SNO-resin-assisted capture technique.

FIGS. 21A-21C. Representative images of tau and NeuN, corresponding to main figures. Images shown are lower power fields from which higher power figures shown in the corresponding main figures were derived, with the higher power field shown in white box. (scale bar=20 μm for original image, 5 μm for inset).

(FIG. 21A) Images for salsalate experiment in FIG. 3C.

(FIG. 21B) Images for Wld^{S} experiments in FIG. 4C.

(FIG. 21C) Images for P7C3-A20 experiments in FIG. 5D.

FIGS. 22A-22D. Salsalate treatment did not affect average speed in behavioral testing or body weight.

(FIG. 22A) Treatment of salsalate improved learning as measured by primary latency during the training period of the Barnes maze. (**p<0.01 vs. Sham-Injury+Vehicle group, or Sham-Injury+Salsalate group, ###p<0.001 vs. TBI+Vehicle group, Repeated-measures two-way ANOVA for learning test).

(FIG. 22B) Body weight was not altered by treatment with salsalate.

(FIG. 22C), (FIG. 22D) Average speed was not affected by treatment with salsalate.

FIG. 23. Validation of plasma ac-tau using wild-type and tau knockout blood samples.

FIG. 24. Western blot for ac-tau in control (Normal) and TBI samples (all others). Subject #260 was removed from the analysis due to the comorbid factor of acute myositis.

FIG. 25. Longitudinal trends of ac-tau during the acute stages of TBI. TBI samples were obtained from 5 time points: T₁, T₃, T₄, T₅ and T₆. Since the availability of samples after T₁ was sparse, samples collected at T₁, samples collected at T₃ and T₄, and samples collected at T₅ and T₆ were grouped together as <24 hours phase, 24-120 hour phase, and >120 hours phase, respectively. Samples included mild (Glasgow Coma Score of 13 to 15), moderate ((Glasgow Coma Score of 9 to 12) and severe head injury (Glasgow Coma Score less than 8). After grouping, there were 44 (15 mild, 15 moderate, 14 severe), 28 (10 mild, 12 moderate, 6 severe) and 17 (5 mild, 2 moderate and 10 severe) samples at <24 hours, 24-120 hours and at >120 hours respectively. One sample at T₃ was discarded as the subject had a sample at T₄ as well. The mean values were fit by splines for better visualization. We observed a decreasing trend in fold-rise of ac-tau values from the acute onset of injury (<24 hour phase) to progression into the sub-acute stages of injury (24-120 hour phase and >120 hour phase).

FIG. 26. Scatter plot of plasma ac-tau with age. There were no significant associations between age and the fold-rise in a, ac-tau (R=−0.21, p=0.08).

FIG. 27. No significant tau seeding activity is detected in acute TBI plasma samples compared to controls. Plasma samples from acute TBI (n=13) and control cases (n=7) were analyzed by AD RT-QuIC. As a positive control, about 18 ng of synthetic fibrils (sFibril) was spiked into one of the control plasma samples, which was then used to seed the assay. Each data point indicates an individual well, analyzed in triplicate for each biological replicate. Lag time is the time required for the ThT fluorescence to exceed a threshold of 100×S.D. above the assay baseline before the assay cutoff, set at 15 h based on lag times to spontaneous amyloid formation in the negative controls.

FIG. 28. Western blot for ac-tau in control (Normal) and SAH samples (all others).

FIG. 29. Western blot for ac-tau in control (Normal) and ICH samples (all others).

### DETAILED DESCRIPTION

Traumatic brain injury (TBI) is a neurodegenerative condition resulting from various forms of head injury. It is a leading cause of mortality and progressive life-long morbidity, which harms the health and quality of life of millions of people worldwide. It is also a major under-appreciated cause of Alzheimer's disease (AD). Unfortunately, no treatment is available, and molecular understanding is lacking. Here, we address these limitations by providing new molecular insights, a novel clinical biomarker, and drugs that stop TBI-induced neurodegeneration.

Specifically, we discovered that S-nitrosylation of GAPDH drives neuronal tau acetylation after TBI through non-canonical activity. Acetylated tau causes breakdown of the axon initial segment, mislocalization of tau from axons into the somatodendritic compartment, axonal degeneration, and cognitive impairment. Drugs that specifically block S-nitrosylation of GAPDH or its downstream consequences on tau acetylation are highly protective in TBI. Finally, acetylated tau in the blood provides a novel biomarker for TBI in both mice and human patients.

The same sites of tau acetylation we observe in TBI have also been noted to be acetylated in the brains of patients with AD, but with unknown function. Thus, our work provides novel mechanistic insight into the potential role of tau acetylation in AD. Since TBI is the largest non-genetic and non-age-related risk factor for AD, our work also likely explains a pathophysiologic convergence between these two disorders.

Disclosed herein, among other things, is the surprising finding that neurodegenerative diseases such as traumatic brain injury (TBI) rapidly increase neuronal acetylated-Tau (Ac-Tau), which then initiates pathological processes that promote neurodegeneration. This rapid Tau acetylation is elicited by nitric oxide-mediated S-nitrosylation, which inhibits Sirtuin1 (Sirt1) deacetylase and induces glyceraldehyde-3-phosphate dehydrogenase to activate p300/CBP acetyltransferase. Elevated Ac-Tau destroys axon initial segments, leading to somatodendritic Tau mislocalization and neurodegeneration. Unexpectedly, elevation of Ac-Tau in the blood is found herein to be a biomarker of neurodegenerative diseases such as TBI in both mouse and human, and that this occurs in both species without Tau attaining seeding ability. In mice, inhibiting p300/CBP or elevating nicotinamide adenine dinucleotide (NAD⁺) to stimulate Sirt1 both reduce levels of Ac-Tau in neurons and blood, and also protect from neurodegeneration and cognitive deficits after TBI. Equivalent protection is observed in mutant mice with constitutively higher levels of NAD⁺. Ac-Tau thus represents a new promising therapeutic target and blood biomarker for neurodegenerative diseases such as traumatic brain injury.

In one aspect, disclosed herein is a method of diagnosing and/or prognosing a neurodegenerative disease in a subject, the method comprising: obtaining a plasma or blood sample from a subject; and detecting a level of acetylated Tau in the plasma or blood sample, wherein a level of acetylated Tau that is at least 25% or 50% higher than a control level in a healthy subject indicates that the subject has a neurodegenerative disease such as traumatic brain injury.

In some embodiments, the obtaining step comprises obtaining a plasma sample from the subject. In some embodiments, the method further comprises depleting albumin and immunoglobulin from the plasma sample. In some embodiments, the method does not involve any brain biopsy sample.

In some embodiments, the detecting step comprises using an antibody or antigen-binding fragment thereof that specifically binds acetylated Tau. In some embodiments, the antibody is a polyclonal antibody. In some embodiments, the antibody is a monoclonal antibody.

Another aspect relates to a method of treating a neurodegenerative disease in a subject, the method comprising administering a low, non-anti-neuroinflammatory dose of salsalate to a subject having a neurodegenerative disease, wherein said dose is 50% or less than an anti-neuroinflammatory dose.

In some embodiments, the low, non-anti-neuroinflammatory dose is about 10-25 mg/kg/day. In some embodiments, the anti-neuroinflammatory dose is about 50 mg/kg/day.

In some embodiments, the method further comprises co-administering an effective amount of 3,6-dibromo-β-fluoro-N-(3-methoxyphenyl)-9H-carbazole-9-propanamine to the subject.

In some embodiments, the method further comprises co-administering an effective amount of CGP3466B (Omigapil) to the subject.

As shown in FIG. 8, reducing brain injury-induced neuronal tau acetylation at any of multiple points in a non-canonical signaling cascade is neuroprotective, with the extent of neurodegeneration being reflected by corresponding blood levels of acetylated-tau. After brain injury, S-nitrosylated (SNO) GAPDH simultaneously activates p300/CBP acetyltransferase and inhibits Sirt1 deacetylase, which together increase the magnitude of neuronal acetylated-tau (ac-tau). This leads to degradation of the axon initial segment, tau mislocalization from the axon into the somatodendritic compartment, and neurodegeneration in the form of axonal degradation and loss of normal synaptic connections, leading to neurobehavioral impairment. Three points of therapeutic intervention are shown: (1) inhibition of GAPDH S-nitrosylation by CGP3466B (omigapil), (2) p300/CBP inhibition by salsalate or diflunisal, and (3) Sirt1 stimulation through elevated nicotinamide adenine dinucleotide (NAD⁺), achieved either pharmacologically with P7C3-A20 or genetically in Wld^{S} mice. Levels of ac-tau in the blood correspond to the magnitude of neurodegeneration in the brain after injury.

### Definitions

Unless defined otherwise, all technical and scientific terms used herein have the meaning commonly understood by one of ordinary skill in the art to which the disclosure pertains. The Definitions section at paragraphs [1001]-[1031] of U.S. Publication No. 2013/0040977 is incorporated herein by reference. Specific terminology is defined below.

As used in the specification and the appended claims, the singular forms “a,” “an,” and “the” include plural referents unless the context clearly dictates otherwise.

As used herein, the term “about” means within 20%, more preferably within 10% and most preferably within 5%.

The terms “neuroprotective” and “neuroprotective activity” refer to an activity in promoting the survival, health, integrity, growth, development and/or function of neurons, and/or protecting neurons from cell death, apoptosis and/or degeneration, and/or stimulating neurogenesis, particularly CNS, brain, cerebral, and hippocampal neurons.

The term “neurogenesis” refers to the process by which neurons are generated from neural stem cells and progenitor cells, which is responsible for populating the growing brain with neurons. While neurogenesis generally is most active during pre-natal development, in some embodiments the compounds disclosed herein can stimulate or promote post-natal neurogenesis such as hippocampal neurogenesis.

The terms “treating” and “treatment” as used herein refer to reduction in severity and/or frequency of symptoms, elimination of symptoms and/or underlying cause, and improvement or remediation of damage.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing and/or that it is included on the Inactive Ingredient Guide prepared by the U.S. Food and Drug administration.

As used herein, the term “patient” or “individual” or “subject” refers to any person or mammalian subject for whom or which therapy is desired, and generally refers to the recipient of the therapy to be practiced according to the disclosure.

A number of small molecules with in vivo neuroprotective properties (the “P7C3 class of compounds”), including P7C3-A20 (3,6-dibromo-β-fluoro-N-(3-methoxyphenyl)-9H-carbazole-9-propanamine), have been previously identified and disclosed in U.S. Pat. No. 8,362,277; U.S. Publication No. 2011/0015217; U.S. Publication No. 2012/0022096; U.S. Publication No. 2013/0040977 and U.S. Application No. 14/339,772 filed July 24, 2014, all of which are hereby incorporated herein by reference in their entirety, in particular the compounds disclosed in the Examples section. It should be noted that while the Examples used P7C3-A20, a number of other P7C3 class of compounds can be used in place of or in combination with P7C3-A20 as a therapeutic agent.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

### Tau and Ac-Tau

“Tau” protein is expressed in central nervous system and plays a critical role in the neuronal architecture by stabilizing intracellular microtubule network. Impairment of the physiological role of the tau protein either by truncation, hyperphosphorylation or by disturbing the balance between the six naturally occurring tau isoforms leads to the formation of neurofibrillary tangles (NFT), dystrophic neurites and neuropil threads. These structures represent ultrastructural hallmarks of Alzheimer's Disease (AD). The major protein subunit of these structures is microtubule associated protein Tau. The amount of NFT found in autopsies of AD patients correlates with clinical symptoms including intellectual decline. Therefore, Tau protein plays a critical role in AD pathology. The recent discovery of co-segregation of specific mutations in the Tau gene with the disease frontotemporal dementia with Parkinsonism linked to chromosome 17 (FTDP-17) has confirmed that certain abnormalities in the Tau protein can be a primary cause of neurodegeneration and dementia in affected individuals.

Tau amino acid sequences are known in the art. See, e.g., the amino acid sequences found under the GenBank accession numbers in parentheses in the following: Human Tau transcript variant 1 mRNA (NM_016835.3) and isoform 1 protein (NP_058519.2); human Tau transcript variant 2 mRNA (NM_005910.4) and isoform 2 protein (NP_005901.2); human Tau transcript variant 3 mRNA (NM_016834.3) and isoform 3 protein (NP_058518.1); human Tau transcript variant 4 mRNA (NM_016841.3) and isoform 4 protein (NP_058525.1); human Tau transcript variant 5 mRNA (NM_001123067.2) and isoform 5 protein (NP_001116539.1); and human Tau transcript variant 6 mRNA (NM_001123066.2) and isoform 6 protein (NP_001116538.1).

Exemplary Tau amino acid sequences include SEQ ID NOs:1-6: Homo sapiens Tau isoform 2 (GenBank Accession No. NP_005901; SEQ ID NO:1); Homo sapiens Tau isoform 3 (GenBank Accession No. NP_058518; SEQ ID NO:2); Homo sapiens Tau isoform 4 (GenBank Accession No. NP_058525; SEQ ID NO:3); Homo sapiens Tau isoform 5 (GenBank Accession No. NP_001116539; SEQ ID NO:4); Homo sapiens Tau isoform 1 (GenBank Accession No. NP_058519; SEQ ID NO:5); and Homo sapiens Tau isoform 6 (GenBank Accession No. NP_001116538; SEQ ID NO:6).

| | | |
|---|---|---|
| seq_id_1 | MAEPRQEFEVMEDHAGTYGLGDRKDQGGYTHHQDQEGDTDAGLKESPLQTPTEDGSERPG | 60 |
| seq_id_2 | MAEPRQEFEVMEDHAGTYGLGDRKDQGGYTMHQDQEGDTDAGLK---------------- | 44 |
| seq_id_3 | MAEPRQEFEVMEDHAGTYGLGDRKDQGGYTMHQDQEGDTDAGLK---------------- | 44 |
| seq_id_4 | MAEPRQEFEVMEDHAGTYGLGDRKGQGGYTMHQDQEGDTDAGLKESPLQTPTEDGSEEPG | 60 |
| seq_id_6 | MAEPRQEFEVMEDHAGTYGLGDRKDQGGYTMHQDQEGDTDAGLKESPLQTPTEDGSEEPG | 60 |
| seq_id_5 | MAEPRQEFEVMEDHAGTYGLGDRKDQGGYTMHQDQEGDTDAGLKESPLQTPTEDGSEEPG | 60 |
| | ******************************************** | |
| seq_id_1 | SETSDAKSTPTAEDVTAPLVDEGAPGKQAAAQPHTEIFEGTTAEEAGIGDTPSLEDEAAG | 120 |
| seq_id_2 | ------------------------------------------AEEAGIGDTPSLEDEAAG | 62 |
| seq_id_3 | ------------------------------------------AEEAGIGDTPSLEDEAAG | 62 |
| seq_id_4 | SETSDAKSTP-----------------------------TAEAEEAGIGDTPSLEDEAAG | 91 |
| seq_id_6 | SETSDAKSTPTAEDVTAPLVDEGAPGKQAAAQPHTEIFEGTTAEEAGIGDTPSLEDEAAG | 120 |
| seq_id_5 | SETSDAKSTPTAEDVTAPLVDEGAPGKQAAAQPHTEIFEGTTAEEAGIGDTPSLEDEAAG | 120 |
| | ****************** | |
| seq_id_1 | HVTQ-------------------------------------------------------- | 124 |
| seq_id_2 | HVTQ-------------------------------------------------------- | 66 |
| seq_id_3 | HVTQ-------------------------------------------------------- | 66 |
| seq_id_4 | HVTQ-------------------------------------------------------- | 95 |
| seq_id_6 | HVTQEPESGKVVQEGFLREPGPPGLSHQLMSGMPGAPLLPEGPREATRQPSGTGPEDTEG | 180 |
| seq_id_5 | HVTQEPESGKVVQEGFLREPGPPGLSHQLMSGMPGAPLLPEGPREATRQPSGTGPEDTEG | 180 |
| | **** | |
| seq_id_1 | ------------------------------------------------------------ | |
| seq_id_2 | ------------------------------------------------------------ | |
| seq_id_3 | ------------------------------------------------------------ | |
| seq_id_4 | ------------------------------------------------------------ | |
| seq_id_6 | GRHQPELLKHQLLGDLHQEGPPLKGAGGKERPGSKEEVDEDRDVDESSPQDSPPSKASPA | 240 |
| seq_id_5 | GRHQPELLKHQLLGDLHQEGPPLKGAGGKERPGSKEEVDEDRDVDESSPQDSPPSKASPA | 240 |

A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of about 350 amino acids of any one of the amino acid sequences set forth in SEQ ID NOs:1-6. A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 350 amino acids to 383 amino acids of the amino acid sequence set forth in SEQ ID NO:2 (Homo sapiens Tau isoform 3). A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 350 amino acids to about 412 amino acids of the amino acid sequence set forth in SEQ ID NO:4 (Homo sapiens Tau isoform 5). A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 350 amino acids to about 400 amino acids, or from about 400 amino acids to about 441 amino acids, of the amino acid sequence set forth in SEQ ID NO:1 (Homo sapiens Tau isoform 2). A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 350 amino acids to about 400 amino acids, from about 400 amino acids to about 500 amino acids, from about 500 amino acids to about 600 amino acids, from about 600 amino acids to about 700 amino acids, or from about 700 amino acids to about 758 amino acids, of the amino acid sequence set forth in SEQ ID NO:5 (Homo sapiens Tau isoform 1). A Tau polypeptide can comprise an amino acid sequence having at least about 75%, at least about 80%, at least about 85%, at least about 90%, at least about 95%, at least about 98%, at least about 99%, or 100%, amino acid sequence identity to a contiguous stretch of from about 350 amino acids to about 400 amino acids, from about 400 amino acids to about 500 amino acids, from about 500 amino acids to about 600 amino acids, from about 600 amino acids to about 700 amino acids, or from about 700 amino acids to about 776 amino acids, of the amino acid sequence set forth in SEQ ID NO:6 (Homo sapiens Tau isoform 6).

In 2010, Dr. Gan group found that acetylation of human tau at lysine 163, 174, and 180 slows tau turnover by inhibiting its ubiquitination, which contributes to tauopathy in Alzheimer's disease (Min S W, Cho S H, Zhou Y, et al. Acetylation of tau inhibits its degradation and contributes to tauopathy [published correction appears in Neuron. 2010 Nov. 18; 68(4):801]. Neuron. 2010; 67(6):953-966. doi: 10.1016/j.neuron.2010.08.044).

In 2011, Dr. Lee group demonstrated that human tau acetylation at lysine 280 inhibits tau function via impaired tau-microtubule interactions and promotes pathological tau aggregation (Cohen T J, Guo J L, Hurtado D E, et al. The acetylation of tau inhibits its function and promotes pathological tau aggregation. Nat Commun. 2011; 2:252. doi: 10.1038/ncomms1255).

In 2013, Dr. Petrucelli group showed that acetylation of tau on KXGS motifs (human lysine 259, 353) inhibits phosphorylation on this same motif (human serine 262, 356), and also prevents tau aggregation (Cook C, Carlomagno Y, Gendron T F, et al. Acetylation of the KXGS motifs in tau is a critical determinant in modulation of tau aggregation and clearance. Hum Mol Genet. 2014; 23(1):104-116. doi:10.1093/hmg/ddt402).

In 2015, Dr. Gan group identified tau acetylation at lysine 174 as an early change in Alzheimer's disease brains and a critical determinant in tau homeostasis and toxicity in mice (Min S W, Chen X, Tracy T E, et al. Critical role of acetylation in tau-mediated neurodegeneration and cognitive deficits. Nat Med. 2015; 21(10):1154-1162. doi:10.1038/nm.3951).

In 2015, Dr. Mucke group identified acetylation sites in wild type and human amyloid precursor protein overexpressing mice by mass spectrometry. They found that endogenous tau at lysine 163, 225, 259, 281, 290, 298, 311, 317, 321, 331, 343, 347, 369, 385 (human tau) are acetylated (Meaghan et al. Tau post-translational modifications in wild-type and human amyloid precursor protein transgenic mice. Nature Neuroscience. 2015).

In 2016, Dr. Gan group reported that tau acetylation at lysine 274, 281 is elevated in human Alzheimer's disease brains. Transgenic mice expressing human tau with lysine-to-glutamine mutations (K274Q, K281Q, mimic acetylation) exhibit AD-related memory deficits and decreased synaptic Kidney/BRAin (KIBRA), a momory-assciated protein, levels (Tracy TE, Sohn P D, Minami S S, et al. Acetylated Tau Obstructs KIBRA-Mediated Signaling in Synaptic Plasticity and Promotes Tauopathy-Related Memory Loss. Neuron. 2016; 90(2):245-260. doi:10.1016/j.neuron.2016.03.005). Also, they found that levels of axon initial segment cytoskeletal proteins are downregulated in human AD brains and correlate negatively with ac-K274 and ac-K281 tau levels (Sohn P D, Tracy T E, Son H I, et al. Acetylated tau destabilizes the cytoskeleton in the axon initial segment and is mislocalized to the somatodendritic compartment. Mol Neurodegener. 2016; 11(1):47. Published 2016 Jun. 29. doi: 10.1186/s13024-016-0109-0).

In 2017, Dr. Cook group showed that acetylation of tau at lysine 321 (within a KCGS motif) inhibits tau aggregation in vitro and prevents tau phosphorylation at serine 324 (Carlomagno Y, Chung D C, Yue M, et al. An acetylation-phosphorylation switch that regulates tau aggregation propensity and function. J Biol Chem. 2017; 292(37): 15277-15286. doi:10.1074/jbc.M117.794602).

In 2018, Dr. Ross group showed overlay of acetylated tau (K280) and phosphorylated tau (MC1) in brain tissues of chronic traumatic encephalopathy patients (Lucke-Wold B, Seidel K, Udo R, et al. Role of Tau Acetylation in Alzheimer's Disease and Chronic Traumatic Encephalopathy: The Way Forward for Successful Treatment. J Neurol Neurosurg. 2017; 4(2): 140).

In 2018, Dr. Sen group demonstrated that amyloid beta-induced s-nitrosylation of GAPDH augments pathological tau acetylation (K280) through activation of acetyltransferase p300 and deactivation of deacetylase SIRT1 (Sen T, Saha P, Sen N. Nitrosylation of GAPDH augments pathological tau acetylation upon exposure to amyloid-β. Sci Signal. 2018; 11(522): eaao6765. Published 2018 Mar 20. doi: 10.1126/scisignal.aao6765).

| Human tau 441 | Mouse tau 430 |
|---|---|
| isoform | isoform |
| K163 | K152 |
| K174 | K163 |
| K180 | K169 |
| K225 | K214 |
| K259 | K248 |
| K274 | K263 |
| K280 | K269 |
| K281 | K270 |
| K290 | K279 |
| K298 | K287 |
| K311 | K300 |
| K317 | K306 |
| K321 | K310 |
| K331 | K320 |
| K343 | K332 |
| K347 | K336 |
| K353 | K342 |
| K369 | K358 |
| K385 | K374 |

Disclosed herein, among other things, is the surprising finding that neurodegenerative diseases such as traumatic brain injury (TBI) rapidly increase neuronal acetylated-Tau (Ac-Tau), which then initiates pathological processes that promote neurodegeneration. This rapid Tau acetylation is elicited by nitric oxide-mediated S-nitrosylation, which inhibits Sirtuin1 (Sirt1) deacetylase and induces glyceraldehyde-3-phosphate dehydrogenase to activate p300/CBP acetyltransferase. Elevated Ac-Tau destroys axon initial segments, leading to somatodendritic Tau mislocalization and neurodegeneration. Unexpectedly, elevation of Ac-Tau in the blood is found herein to be a biomarker of neurodegenerative diseases such as TBI in both mouse and human, and that this occurs in both species without Tau attaining seeding ability. In mice, inhibiting p300/CBP or elevating nicotinamide adenine dinucleotide (NAD⁺) to stimulate Sirt1 both reduce levels of Ac-Tau in neurons and blood, and also protect from neurodegeneration and cognitive deficits after TBI. Equivalent protection is observed in mutant mice with constitutively higher levels of NAD⁺. Ac-Tau thus represents a new promising therapeutic target and blood biomarker for neurodegenerative diseases such as traumatic brain injury. In some embodiments, antibodies can be made against one or more acetylation sites on Tau as disclosed above, using methods known in the art.

### Pharmaceutical Compositions

The term “pharmaceutically acceptable carrier” refers to a carrier or adjuvant that may be administered to a subject (e.g., a patient), together with a compound of the present disclosure, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

The amount administered depends on the compound formulation, route of administration, etc. and is generally empirically determined in routine trials, and variations will necessarily occur depending on the target, the host, and the route of administration, etc. Generally, the quantity of active compound in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The following are examples (Formulations 1-4) of capsule formulations.

Capsule Formulations

| Capsule | Formulation 1 | Formulation 2 | Formulation 3 | Formulation 4 |
|---|---|---|---|---|
| Formulation | mg/capsule | mg/capsule | mg/capsule | mg/capsule |
| Compound (solid | 100 | 400 | 400 | 200 |
| solution) | | | | |
| Silicon Dioxide | 0.625 | 2.5 | 3.75 | 1.875 |
| Magnesium | 0.125 | 0.5 | 0.125 | 0.625 |
| Stearate NF2 | | | | |
| Croscarmellose | 11.000 | 44.0 | 40.0 | 20.0 |
| Sodium NF | | | | |
| Pluronic F68 NF | 6.250 | 25.0 | 50.0 | 25.0 |
| Silicon Dioxide NF | 0.625 | 2.5 | 3.75 | 1.875 |
| Magnesium | 0.125 | 0.5 | 1.25 | 0.625 |
| Stearate NF | | | | |
| Total | 118.750 | 475.00 | 475.00 | 475.00 |
| Capsule Size | No. 4 | No. 0 | No. 0 | No. 2 |

### Preparation of Solid Solution

Crystalline compound (80 g/batch) and the povidone (NF K29/32 at 160 g/batch) are dissolved in methylene chloride (5000 mL). The solution is dried using a suitable solvent spray dryer and the residue reduced to fine particles by grinding. The powder is then passed through a 30 mesh screen and confirmed to be amorphous by x-ray analysis.

The solid solution, silicon dioxide and magnesium stearate are mixed in a suitable mixer for 10 minutes. The mixture is compacted using a suitable roller compactor and milled using a suitable mill fitted with 30 mesh screen. Croscarmellose sodium, Pluronic F68 and silicon dioxide are added to the milled mixture and mixed further for 10 minutes. A premix is made with magnesium stearate and equal portions of the mixture. The premix is added to the remainder of the mixture, mixed for 5 minutes and the mixture encapsulated in hard shell gelatin capsule shells.

### Use

In one aspect, methods for treating (e.g., controlling, relieving, ameliorating, alleviating, or slowing the progression of) or methods for preventing (e.g., delaying the onset of or reducing the risk of developing) one or more diseases, disorders, or conditions. The methods include administering to the subject an effective amount of any compound described herein or a salt (e.g., a pharmaceutically acceptable salt) thereof as defined anywhere herein to the subject.

In another aspect, the use of any compound described herein or a salt (e.g., a pharmaceutically acceptable salt) thereof as defined anywhere herein in the preparation of, or for use as, a medicament for the treatment (e.g., controlling, relieving, ameliorating, alleviating, or slowing the progression of) or prevention (e.g., delaying the onset of or reducing the risk of developing) of one or more diseases, disorders, or conditions.

In embodiments, the one or more diseases, disorders, or conditions can include neuropathies, nerve trauma, and neurodegenerative diseases. In embodiments, the one or more diseases, disorders, or conditions can be diseases, disorders, or conditions caused by, or associated with aberrant (e.g., insufficient) neurogenesis (e.g., aberrant hippocampal neurogenesis as is believed to occur in neuropsychiatric diseases) or accelerated death of existing neurons. Examples of the diseases, disorders, or conditions include, but are not limited to, DNA-damaging agent (e.g., anthracycline) mediated cardiotoxicity, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

The resultant promotion of neurogenesis or survival of existing neurons (i.e., a resultant promotion of survival, growth, development, function and/or generation of neurons) may be detected directly, indirectly or inferentially from an improvement in, or an amelioration of one or more symptoms of the disease or disorder caused by or associated with aberrant neurogenesis or survival of existing neurons. Suitable assays which directly or indirectly detect neural survival, growth, development, function and/or generation are known in the art, including axon regeneration in rat models (e.g. Park et al., Science. 2008 Nov. 7; 322:963-6), nerve regeneration in a rabbit facial nerve injury models (e.g. Zhang et al., J Transl Med. 2008 Nov. 5; 6(1):67); sciatic nerve regeneration in rat models (e.g. Sun et al., Cell Mol Neurobiol. 2008 Nov. 6); protection against motor neuron degeneration in mice (e.g. Poesen et al., J. Neurosci. 2008 Oct. 15; 28(42): 10451-9); rat model of Alzheimer's disease, (e.g. Xuan et al., Neurosci Lett. 2008 Aug. 8; 440(3):331-5); animal models of depression (e.g. Schmidt et al., Behav Pharmacol. 2007 September; 18(5-6):391-418; Krishnan et al., Nature 2008, 455, 894-902); and/or those exemplified herein.

### Administration

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, New York, 537 (1970). In certain embodiments, the compositions are administered by oral administration or administration by injection. The methods herein contemplate administration of an effective amount of compound or compound composition to achieve the desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

In some embodiments, the compounds described herein can be co-administered with one or more other therapeutic agents. In certain embodiments, the additional agents may be administered separately, as part of a multiple dose regimen, from the compounds of the present disclosure (e.g., sequentially, e.g., on different overlapping schedules with the administration of one or more compounds disclosed herein (including any subgenera or specific compounds thereof)). In other embodiments, these agents may be part of a single dosage form, mixed together with the compounds of the present disclosure in a single composition. In still another embodiment, these agents can be given as a separate dose that is administered at about the same time that one or more compounds disclosed herein (including any subgenera or specific compounds thereof) are administered (e.g., simultaneously with the administration of one or more compounds disclosed herein (including any subgenera or specific compounds thereof)).

When the compositions of the present disclosure include a combination of a compound described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compositions of the present disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a compound of the present disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the compounds of the present disclosure include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of the present disclosure may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in the present disclosure. Also within the present disclosure is a patch to deliver active chemotherapeutic combinations herein. A patch includes a material layer (e.g., polymeric, cloth, gauze, bandage) and the compound delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compounds disclosed herein and an additional agent (e.g., a therapeutic agent) can be administered using any of the routes of administration described herein. In some embodiments, a composition having the compound disclosed herein and an additional agent (e.g., a therapeutic agent) can be administered using an implantable device. Implantable devices and related technology are known in the art and are useful as delivery systems where a continuous or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compounds and compositions delineated herein.

The present disclosure will be further described in the following examples. It should be understood that these examples are for illustrative purposes only and are not to be construed as limiting the present disclosure in any manner.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); wt, wild type; and the like.

### Example 1: Reducing Tau Acetylation is Neuroprotective

### Summary

Traumatic brain injury (TBI) is the largest non-genetic, non-aging-related risk factor for Alzheimer's disease (AD). We report that TBI induces acetylation of neuronal tau (ac-tau) at sites acetylated also in AD brains, and that this is mediated by non-canonical activity of S-nitrosylated-GAPDH to inactivate Sirtuin1 deacetylase while also acetylating and activating p300/CBP acetyltransferase, thereby coordinately increasing ac-tau. Ac-tau mislocalizes to cause neurodegeneration leading to neurobehavioral impairment, and also accumulates in serum of humans and mice following TBI. Drugs that block GAPDH S-nitrosylation, inhibit p300/CBP activity, or enhance Sirtuin1 activity independently protect mice from neurodegeneration, neurobehavioral impairment, and accumulation of ac-tau in brain and plasma after brain injury. Strikingly, patients receiving the p300/CBP inhibitors salsalate or diflunisal exhibit decreased incidence of both AD and clinically-diagnosed TBI. Thus, ac-tau is a therapeutic target and peripheral biomarker of brain injury. We hypothesize that ac-tau may represent molecular pathologic convergence between TBI and AD.

### Introduction

Traumatic brain injury (TBI) is typically caused by motor vehicle crashes, falls, contact sports, or assaults. The annual incidence of TBI in the United States alone is about 3.5 million, with about 5 million people currently living with TBI-related disabilities at an annual cost of about $80 billion (Centers for Disease Control and Prevention, 2015; Ma et al., 2014). At present, treatments for TBI focus on patient stabilization and mitigation of symptoms, and there are no medicines that specifically target the pathophysiological processes that drive neurodegeneration after brain injury. TBI also significantly increases the risk of later developing Alzheimer's disease (AD) (Johnson et al., 2010; Li et al., 2017). This suggests common pathologic mechanisms, and emerging evidence points to S-nitrosylation and acetylation (Uehara et al., 2006; Nakamura & Lipton, 2020; Sen et al., 2018). Indeed, a small post-mortem study recently reported increased acetylation of tau at lysine (K) 280 in the brains of three patients with AD and three patients with chronic traumatic encephalopathy (Lucke-Wold et al., 2017). In a separate study, the same rise in ac-tau at K280 was documented in the brains of ten additional patients with AD, five patients with corticobasal degeneration, and five patients with progressive supranuclear palsy (Irwin et al., 2012). Tau acetylation at both K274 and K281 was additionally reported in upwards of a dozen patients with severe AD (Tracy et al., 2016). However, these studies did not establish the driving forces or pathologic significance of the findings. Given the interrelationship between TBI and AD, we sought to determine whether elevated ac-tau was a causal pathophysiologic factor in TBI, and thus a potential locus of convergence, and if so then whether this might provide an experimental platform to understand in vivo pathophysiology related to ac-tau in the brain.

### Results

### Brain Injury Induces Neuronal Tau Acetylation

To begin, we employed a mouse model of multimodal TBI incorporating concussive impact, acceleration/deceleration, and early phase blast wave exposure (Extended Methods) (Shin and Vázquez-Rosa et al, 2021). This model produces a complex brain injury with neurodegeneration and neurobehavioral impairment, beginning with acute axonal degeneration that persists chronically and results in quantifiable blood brain barrier degradation and nerve cell death over a year after injury (Yin et al., 2014, 2016; Dutca et al., 2014; Vázquez-Rosa et al., 2019; Wattiez et al., 2020; Vázquez-Rosa, Shin, Dhar et al., 2020). Already an established preclinical model of TBI, we have now further strengthened its clinical relevance through nonbiased whole metabolomic analysis. Specifically, one week after injury, mice display altered plasma levels of fatty acids, amino acids, 5-oxoproline, and TCA cycle metabolites (FIG. 9), which resembles published data from nonbiased plasma metabolomic analysis from human TBI patients (Oresic et al., 2016).

Using antibodies to mouse acetylated-tau (ac-tau) at K263 and K270 (Min et al., 2010) (FIG. 10), which correspond to human tau K274 and K281, we observed rapid and specific elevation of ac-tau in both the cerebral cortex and hippocampus of the brain after injury (FIG. 1A). Importantly, the increased acetylation was observed selectively in neurons of the brain (FIG. 1B) and correlated with injury intensity (FIG. 1C), while total tau levels remained unchanged (FIGS. 1A-1C). Our observation of the low-level presence of tau in non-neuronal cell types is consistent with previous reports (www.brainrnaseq.org; Pereas et al., 2019; Bolos et al., 2017). This injury-dependent increase in neuronal tau acetylation was observed in both males and females (FIG. 11A), and also generalized to multiple forms and stages of TBI. For example, we determined that this is not only an acute, but also a chronic response to injury, as ac-tau remained elevated 17 months after injury in mice (FIG. 11B). Ac-tau was also elevated seven months after acoustic blast overpressure in rats (FIG. 11C), and at a range of times after controlled cortical impact in mice (FIG. 11D). In addition, we observed that ac-tau is acutely elevated in cerebellum after TBI (FIG. 11E), but not in hypothalamus (FIG. 11F). This is consistent with our previous observation that the hypothalamus is resistant to neurodegeneration in this model of multimodal TBI (Yin et al., 2014), for reasons that are not currently known.

By contrast, tau phosphorylation after brain injury was unchanged at S202 (FIG. 12A and B), which is part of the basis for AD Braak staging (Neddens et al., 2018), and reduced at S396 and S404, sites that mediate tau's ability to polymerize tubulin (Evans et al., 2000) (FIG. 12A and B). Tau phosphorylation was also somewhat reduced at S262 (FIG. 12A and B). Although phosphorylation of S262 has been linked to tau acetylation in other contexts (Cook et al., 2014), inhibiting tau acetylation after brain injury had no effect on S262 phosphorylation (FIG. 12C). Taken together, our data suggest a prominent role for tau acetylation and a lesser role for tau phosphorylation in the acute period following brain injury. Notably, the post-injury increase in ac-tau was also not associated with tau seeding (FIG. 13A and B), nor with reduced expression of postsynaptic Kidney/BRAin (KIBBRA) protein (FIG. 14A-D), a previously suggested consequence of tau acetylation (Tracy et al., 2016).

### Injury-Induced Neuronal Tau Acetylation Leads to Axon Initial Segment Degradation and Pathologic Tau Mislocalization

We uncovered clues to the possible function of ac-tau in brain injury from published post-mortem studies of AD patients' brains, in which the magnitude of K274 and K281 acetylation correlated inversely with amounts of ankyrin-G (AnkG) and βIV-spectrin (Sohn et al., 2016), principle components of the axon initial segment (AIS). The AIS normally maintains neuronal health and limits tau distribution predominantly to axons (Schafer et al., 2009). After injury, we quantified rapid reductions in the levels of AnkG and βIV-spectrin (FIG. 1D), which correlated with amounts and time-course of increased tau acetylation (FIG. 1A). We also observed that AIS degradation led to pathologic tau mislocalization into the somatodendritic compartment of neurons throughout the brain (FIG. 1E).

To determine whether ac-tau was directly toxic to neurons, we conducted both in vitro and in vivo genetic tests. To begin, transfection of cultured human neuroblasts with either human tau acetylation mimetic or acetylation-resistant human tau showed that tau acetylation specifically increased neuronal cell death in an in vitro model of traumatic brain injury, as measured by both cleaved caspase 3 and lactate dehydrogenase (LDH) release assay (Figure IF). Next, we examined mouse correlates of this model using transgenic mice expressing human tau with lysine-to-glutamine mutations that mimic acetylation at K274 and K281, which correspond to mouse K263 and K270. These mice, known as tauKQ^{high} mice, have been previously shown to exhibit memory deficits and impaired long-term potentiation (Tracy et al., 2016). Here, we show that about one-year-old tauKQ^{high} mice display a significant degree of axonal degeneration, which is not observed in either nontransgenic wild-type littermates or human wild-type Tau transgenic mice (FIG. 1G, FIG. 15A and B). The interesting observation that the hypothalamus of tauKQ^{high} mice showed prominently increased axon neurodegeneration contrasts with our previous reports of protection of this region of the brain in TBI (FIG. 12F, and Yin et al., 2014), suggesting that this region may be physically protected from injury relative to other regions of the brain. In addition to axon degeneration, we also assessed synaptic integrity in these animals through immunohistochemical staining for synaptic vesicle protein 2 (SV2). As shown in FIG. 15C, tauKQ^{high} mice display reduced SV2 in the hippocampus relative to nontransgenic wild-type littermates. Taken together, these results indicate that human ac-tau is directly toxic to neurons, both in vitro and in vivo in the brain.

### Injury-Induced Neuronal Tau Acetylation is Driven by Non-Canonical Signaling

In cellular models of AD, protein acetylation has been linked to S-nitrosylation of GAPDH, which coordinately enhances p300/CBP acetyltransferase activity and inhibits Sirtuin1 (Sirt1) deacetylase activity to increase overall amounts of acetylation. Inhibitory S-nitrosylation of Sirt1 is mediated by aberrant transnitrosylase activity of GAPDH, whereas S-nitrosylated-GAPDH (SNO-GAPDH) also stimulates activation and acetylation of p300/CBP acetyltransferase (Hara and Snyder, 2006; Kornberg et al., 2010; Sen et al., 2018). In our mouse model of brain injury, we found that both GAPDH and Sirt1 were S-nitrosylated after TBI (FIG. 2A). Increased protein acetylation was further confirmed by measuring the acetylation of histone H2AK5, a p300/CBP and Sirt1 substrate (FIG. 16A and B). Initiation of daily treatment 15 minutes after injury with CGP3466B (0.014 mg/kg IP), a specific inhibitor of GAPDH nitrosylation (Waldmeier et al., 2000), blocked S-nitrosylation of both GAPDH and Sirt1 (FIG. 2B and FIG. 16C). We also note that protein or activity levels of histone deacetylase 6 (HDAC6), which can deacetylate tau (Cohen et al., 2014; Cook et al., 2014), did not change any of the time points after TBI (FIG. 16D). Blocking GAPDH S-nitrosylation also reduced amounts of ac-tau and ac-p300/CBP (FIG. 2C and FIG. 17A). This treatment additionally blocked degradation of the AIS (FIG. 2D and FIG. 17B), prevented redistribution of tau into the somatodendritic compartment (FIG. 2E and FIG. 17C), and reduced axon degeneration throughout the brain (FIG. 2F). Lastly, we assessed protective efficacy for cognition by assessing mice in the Barnes maze task of learning and memory. Daily treatment with CGP3466B (0.014 mg/kg IP), this time not initiated until 24 hours after injury, protected mice from injury-induced deficits in both learning and memory (FIG. 2G), without altering motor speed (FIG. 17D and 17E) or body weight (FIG. 17F).

Inhibiting p300/CBP Acetyltransferase Blocks Injury-Induced Tau Acetylation, Neurodegeneration, and Neurobehavioral Impairment

Given the multiple mechanistic steps that follow injury-dependent S-nitrosylation of GAPDH to converge on elevating ac-tau, we considered complementary therapeutic approaches. We first tested salsalate, a known inhibitor of rodent and human p300/CBP (Shirakawa et al., 2016). Since salsalate has anti-inflammatory properties, we identified a low dose that inhibited p300/CBP (FIGS. 18A-18D), without blocking elevated levels of neuroinflammatory cytokines after injury (FIGS. 19A and 19B). Daily administration of this low non-anti-inflammatory dose of salsalate, beginning 24 h after TBI, reduced amounts of ac-tau in the brain without changing the total amount of tau protein (FIG. 3A) or GAPDH S-nitrosylation (FIG. 20). This same low-dose salsalate treatment also preserved the AIS after injury, as evidenced by unchanged levels of AnkG and βIV-spectrin (FIG. 3B). Encouragingly, compared with sham-injured animals, low-dose salsalate preserved normal axonal localization of tau (FIG. 3C and FIG. 21A) and protected mice from axonal degeneration (FIG. 3D).

As a final measure of protection, we assessed both motor and cognitive function after injury (FIG. 3E). Notably, daily low-dose, non-anti-inflammatory salsalate treatment initiated 24 hours after injury preserved normal motor function in the foot slip assay (FIG. 3E) and also protected mice from acquiring deficits in learning (FIG. 3E and FIG. 22A) and memory (FIG. 3E) in the Barnes maze task, without impacting body weight (FIG. 22B) or motor speed (FIG. 22C and 22D).

### Elevating NAD⁺ Enhances Sirt1 Activity and Blocks Injury-Induced Tau Acetylation, Neurodegeneration, and Neurobehavioral Impairment

Next, we tested the complementary approach of increasing Sirt1-mediated tau deacetylation. Sirt1 activity is dependent on nicotinamide adenine dinucleotide (NAD⁺), and high expression of the Ube4b/nicotinamide mononucleotide adenylyltransferase 1 fusion protein in Wld^{S} mice results in constitutively elevated NAD⁺ levels that protect them from axon degeneration and neurobehavioral impairment after brain injury (Pieper & McKnight, 2018; Yin et al., 2016). We observed here that Wld mice were also completely protected from injury-induced increases in ac-tau (FIG. 4A) and AIS degradation throughout the brain (FIG. 4B). This protection correlated with complete protection from tau mislocalization as well (FIG. 4C and FIG. 21B).

Neuronal levels of NAD⁺ can also be increased pharmacologically by treatment with the aminopropyl carbazole P7C3-A20 (Pieper et al., 2010; Wang et al., 2014; Pieper & McKnight, 2018). P7C3-A20, administered daily beginning 24 h after brain injury, preserved brain NAD⁺ levels (FIG. 5A) and prevented accumulation of ac-tau (FIG. 5B). This was correlated with protection from injury-induced AIS degradation (FIG. 5C) and mislocalization of tau into the somatodendritic compartment (FIG. 5D and FIG. 21C). Importantly, P7C3-A20-mediated protection was conferred without impacting upstream injury-induced S-nitrosylation of GAPDH after TBI (FIG. 5E). All aspects of P7C3-A20-mediated protection were blocked by inhibiting Sirt1 with EX527 or inhibiting NAD⁺ synthesis with FK866 (FIGS. 5A-5D). Thus, P7C3-A20-mediated preservation of otherwise depleted neuronal NAD⁺ after brain injury promotes downstream Sirt1-mediated deacetylation of tau, as well as AIS degradation and tau mislocalization.

### Acetylated Tau is a Blood Biomarker of Traumatic Brain Injury-Induced Neurodegeneration in Mice and Humans

Since tau freely diffuses from brain to blood, we postulated that ac-tau might serve as a blood biomarker of post-TBI neurodegeneration. After depleting plasma immunoglobulin because of its size overlap with tau, we probed for ac-tau by western blot and observed elevated levels in mouse plasma after TBI (FIG. 6A). Specificity of plasma ac-tau was confirmed by using wild type and tau knockout mice plasma sample (FIG. 23). Notably, the same protective therapies that reduced brain concentrations of ac-tau (treatment with CGP3466B, low-dose salsalate, or P7C3-A20) also decreased its concentration in plasma (FIGS. 6B-6E).

Next, to evaluate for clinical relevance in human patients, we quantified plasma ac-tau levels in TBI patients admitted to a neuroscience intensive care unit of Memorial Herman Hospital-Texas Medical Center (Houston, Texas) from December 2017 to April 2019 (FIG. 24 and Table 1). In these patients, plasma neurofilament light (Nfl), ubiquitin C-terminal hydrolase L1 (UCHL1) and glial fibrillary acidic protein (GFAP), but not phosphorylated tau (pTau181)/Tau were increased relative to control samples (FIG. 6F). We found that plasma ac-tau was also increased by about 50% within Day 1 of admission compared to controls (p<0.0001) (FIG. 6G), and remained consistently elevated across all time points (FIG. 25). Ac-tau levels showed no age- or gender-dependent differences (FIG. 26; for ac-tau female vs male 1.32 vs 1.64, p>0.05), and there was no tau seeding activity in human TBI vs control samples (FIG. 27). Lastly, we observed relative specificity for elevated levels of plasma ac-tau in TBI, relative to plasma samples from human patients with either subarachnoid hemorrhage (SAH) (FIG. 6H and FIG. 28) or intracerebral hemorrhage (ICH) (FIG. 6I and FIG. 29).

NSAIDs That Inhibit p300/CBP are Associated with Decreased Incidence of Clinically-Diagnosed TBI and Alzheimer's Disease in People

In addition to the NSAID salsalate, the related salicylate diflunisal has also been demonstrated to inhibit p300/CBP (Shirakawa et al., 2016). Though diflunisal has much lower blood-brain barrier penetration than salsalate, along the order of 1:100 (Merck & Co, Inc.), it also inhibits p300/CBP acetyltransferase at lower doses than what is typically administered for anti-inflammatory effect. Thus, we wondered whether salsalate or diflunisal usage in people might be neuroprotective, relative to the common NSAID aspirin that does not inhibit p300/CBP (Shirakawa et al., 2016). Notably, a recent randomized placebo-controlled trial in about 19,000 subjects also showed that aspirin use does not significantly reduce the incidence of dementia, minor cognitive impairment, or cognitive decline (Ryan et al., 2020).

Because ac-tau has been generally reported in the brains of subjects with AD, we evaluated the relationship between salsalate or diflunisal use and incidence of clinically-diagnosed TBI or AD by analyzing 7.23 million U.S. commercially-insured individuals (MarketScan Medicare Supplemental database). We conducted two cohort analyses to evaluate the predicted association, based on individual level of longitudinal patient data and pharmacoepidemiologic methods, as previously established (Cheng et al., 2018). Thus, our two cohorts included: (1) salsalate vs. a matched aspirin population and (2) diflunisal vs. a matched aspirin population (Table 2). For each comparison, we estimated the un-stratified Kaplan-Meier curves and conducted propensity score (PS) stratified (n strata=10) log-rank tests and a Cox regression model after adjusting age, race, sex, and disease comorbidities (including diabetes, hypertension, and coronary artery disease) using a propensity score-matching method (Methods). After 6 years of follow-up, salsalate usage was significantly associated with a reduced risk of clinically-diagnosed TBI (hazard ratio [HR]=0.70, 95% confidence interval [CI] 0.55-0.89, p<0.001, FIG. 7A) and AD (HR=0.57, 95% CI 0.42-0.77, p<0.001, FIG. 7B), compared with a PS-matched aspirin cohort by Cox regression model. Strikingly, diflunisal usage was associated with an even greater reduced incidence of clinically-diagnosed TBI (HR=0.61, 95% CI 0.43-0.86, p=0.003, FIG. 7A). Moreover, usage of either salsalate or diflunisal was also associated with reduced incidence of AD, with the effect again more pronounced in diflunisal than salsalate (HR 0.17, 95% CI 0.08-0.36, p<0.001, FIG. 7B), compared with a PS-matched aspirin cohort. The greater efficacy of diflunisal relative to salsalate correlates with the greater potency of diflunisal to inhibit p300/CBP. Lastly, to more specifically control for co-morbid factors associated with AD, we performed an additional subgroup analysis in which all subjects with diabetes, hypertension, and coronary artery disease were removed, and again confirmed that either salsalate or diflunisal usage is associated with a reduced incidence of AD, with much stronger effect seen in patients who received diflunisal (FIG. 7C).

Because diflunisal is an even more potent inhibitor of p300/CBP than salsalate, we wondered whether it might also inhibit the accumulation of ac-tau after brain injury. First, we determined that peripherally administered diflunisal dose-dependently crosses the blood brain barrier in mice, as shown in FIG. 7D. Diflunisal was administered PO and animals were euthanized at the indicated times post-dose. After peripheral blood collection, animals were perfused with 1×PBS to remove blood in the brain vasculature. Total plasma and brain levels of diflunisal were evaluated by LC-MS/MS and then converted to free drug levels after measurement of binding in mouse plasma and brain. In line with previous observations of diflunisal levels in baboon cerebrospinal fluid (CSF) (Merck & Co, Inc), we found relatively modest penetration of diflunisal into mouse brain. On average across the two time points and three concentrations evaluated, the total diflunisal plasma to brain ratio was about 200:1. However, when free drug levels were calculated, the ratio decreased to only 55:1 because the fraction unbound in brain tissue was higher than in plasma (fu_{brain}=0.050±0.001; fuₚₗₐₛₘₐ=0.014±0.002). Furthermore, when absolute concentrations were taken into account, total diflunisal brain levels reached approximately 3.9 μM (972 ng/g), and free levels were 196 nM (49 ng/g) at the 50 mg/kg dose in mice. Total plasma levels at this dose ranged from 167-232 μg/mL in mice, a concentration similar to mean peak levels of 190+33 mcg/mL observed in humans given 500 mg diflunisal twice daily for 11 days (Merck & Co, Inc), confirming the physiological relevance of the doses employed here. Therefore, we evaluated the efficacy of daily diflunisal administration, beginning 24 hours after TBI, to block the expected rise in ac-tau. As shown in FIG. 7D, the accumulation of ac-tau in cortical brain tissue progressively decreased with escalating doses of diflunisal, which was administered daily starting 24 hours after injury. Moreover, this was also reflected in dose-dependent decreases in blood ac-tau as well (FIG. 7E), with tight correlation between plasma and brain levels of ac-tau (FIG. 7F, R=0.857, p<0.0001).

### Discussion

Taken together, our findings reveal critical cross-talk between S-nitrosylation and acetylation after brain injury, which converges on neuronal ac-tau. While previous studies have reported phosphorylated tau in human TBI (Yang et al., 2016; Okamura et al., 2019; Gorgoraptis et al., 2019), these observations were beyond the first 24 hours after injury, which we examined here. Importantly, independent analysis of our 24 hour samples showed no increase in tau phosphorylation. Moreover, our genetic in vitro and in vivo studies show a direct neurotoxic effect of ac-tau on neurons. Thus, our results support a critical role for tau acetylation in the acute period following brain injury, preceding any effects of tau phosphorylation. Future studies will focus on the interplay between tau acetylation and the myriad other pathological post-translational modifications of tau that have been reported, including phosphorylation and ubiquitination (Wesseling et al., 2020).

AD-like pathology in experimental systems had been linked previously to N-methyl-d-aspartic acid-mediated, neuronal nitric oxide synthase-dependent S-nitrosylation (Sen et al., 2018), and separately SNO-GAPDH had been implicated in modulating protein acetylation (Kornberg et al., 2011). However, understanding of how protein S-nitrosylation connected to human-relevant in vivo models of neurodegeneration after brain injury was missing. We now show how this process mechanistically unfolds, and how these insights form the basis of effective therapy. After injury, GAPDH S-nitrosylation leads to ac-tau accumulation and subsequent ac-tau-mediated pathology in neurons. Specifically, brain injury-induced SNO-GAPDH coordinately activates p300/CBP acetyltransferase and inhibits Sirt1 deacetylase to increase amounts of neuronal ac-tau. Drugging SNO-GAPDH with CGP3466B, or p300/CBP with low-dose salsalate or diflunisal, inhibits tau acetylation and downstream consequences of brain injury. Indeed, our observation of a strong epidemiologic association of diflunisal usage with decreased AD in people, coupled with our finding that diflunisal potently blocks accumulation of ac-tau after brain injury in mice, supports the notion of clinical trials of diflunisal for TBI and AD.

On the other side of the balance of acetylation, SNO-GAPDH-mediated inhibition of NAD⁺-dependent Sirt1 deacetylase is mediated by enzymatic S-nitrosylation of Sirt1, thereby preventing tau deacetylation. Maintaining Sirt1 activity through treatment with P7C3-A20, which preserves NAD⁺ levels in injured neurons, also protects against the same deleterious outcomes after brain injury. Thus, multiple lines of evidence converge to show that reducing neuronal levels of ac-tau is a new therapeutic approach for treating TBI, whether that be through inhibiting acetylation with already existing drugs that could be repurposed for TBI, or through enhancing deacetylation through a drug to emerge from the P7C3 series of molecules. Other strategies to preserve NAD⁺ after injury, such as future pharmacologic inhibitors of the NAD⁺ hydrolase SARM1, might be similarly effective (Pieper & McKnight, 2018).

Both inhibition of tau acetylation and activation of tau deacetylation were observed following post-injury treatment with CGP34668, which selectively inhibits S-nitrosylation of GAPDH. CGP34668, also named “omigapil,” is an analog of the irreversible inhibitor of monoamine oxidase B known as deprenyl, which is employed to treat patients with Parkinson's disease and depression. Notably, omigapil has recently shown safety in a Phase 1 clinical trial for patients with pediatric and adolescent congenital muscular dystrophy (CMD; NCT01805024). Abnormally frequent neurofibrillary tangles of tau in the cerebral cortex have been reported in several studies of CMD patients, and variations in tau have also been associated with CMD (Vermersch et al., 1996). Our elucidation of the downstream events in the brain following GAPDH-S-nitrosylation after brain injury may thus provide new strategies to preserve brain health in CMD patients. As tau is also found in muscle fibers (Lubke et al., 1994), it is likewise possible that the signaling cascade and opportunities for therapeutic intervention that we have characterized here in the brain could similarly apply to improving muscular health in patients with CMD.

Lastly, there is a tremendous unmet need for robust biomarkers that can establish whether a head injury has affected the brain, as well as stratify the severity and nature of the brain injury and objectively identify whether it is resolving. For example, a blood-based biomarker could overcome limitation of current neuropsychological tests for brain injury, and also help detect brain trauma that is masked by other injuries or symptoms. Rapid and accurate field diagnosis of brain injury is also critical for assuring that athletes and military personnel are not placed at risk for a second injury before they have fully recovered from the first. While numerous such markers have been proposed in CSF (Zetterberg and Blennow, 2016), collection of peripheral blood samples is considerably easier. However, the low concentration of potential biomarkers in peripheral blood can be technically limiting, and the concentration of brain proteins in the blood can vary as a function of integrity of the BBB. Thus, a robust marker that freely diffuses across the BBB from the brain into the blood, such as tau protein, is desired. Here, we show that ac-tau levels rapidly rise in the blood of both rodents and humans after brain injury, and that serum levels decline proportionally with protective treatments targeted to this signaling cascade. Other proposed brain injury markers in the field include S100-B and GFAP, as serum biomarkers of astroglial injury (Mondello et al., 2018). However, S100-B is also expressed in cells outside of the brain, such as adipocytes and chondrocytes (Olsson et al., 2011). We also show here that detection of ac-tau is a more sensitive early indicator of brain injury than serum GFAP. Another candidate peripheral biomarker from brain injury is neuron specific enolase (NSE), an enzyme involved in glycolytic energy metabolism (Mondello et al., 2018). However, the utility of NSE is limited because erythrocytes and platelets also contain high amounts of this enzyme (Tolan et al., 2013; Geisen et al., 2015). Elevated NSE has also been associated with many tumors, as well as ischemic stroke, intracerebral hemorrhage, and seizures (Isgro et al., 2015). Recently, serum GFAP, Nfl and UCH-L1 have also been proposed as serum biomarkers for TBI (Shahim et al., 2020). Thus, ac-tau now joins a growing list of potential blood biomarkers for neurodegeneration after brain injury. Importantly, ac-tau is the first TBI biomarker that is mechanistically linked to both pathophysiology and a therapeutic treatment strategy. Ac-tau levels in the blood correlate directly with its brain levels, and blood ac-tau appears to distinguish TBI-induced neurodegeneration from other forms of pathology, such as subarachnoid and intracerebral hemorrhage. Whether blood ac-tau levels correlate with the progression and severity of other forms of neurodegeneration, such as AD, remains to be investigated.

In summary, we show that ac-tau is a previously unrecognized contributor to TBI pathophysiology. Tau acetylation sites after brain injury correspond to sites implicated in human AD, reflecting a shared mechanism of aberrant SNO-GAPDH signaling that may serve as a pathophysiologic mechanism for the increased risk of later developing AD after brain injury. The presence of a small degree of baseline ac-tau in the uninjured state prompts future investigation of the possibility for a normal biological role of tau acetylation, with toxicity resulting when acetylation exceeds a threshold. At this time, our results here establish that reducing tau acetylation through multiple different points of therapeutic intervention after brain injury offers a new neuroprotective strategy, and quantifying tau acetylation provides a new peripheral biomarker of traumatic brain injury.

### REFERENCES

Bolos M, Llorens-Martin M, Perea JR, Jurado-Arjona J, Rabano A, Hernandez F, and Avila J. (2017). Absence of CX3CR1 impairs the internalization of Tau by microglia. Mol. Neurodegener. 12: 59.
Centers for Disease Control and Prevention. (2015). Report to congress on traumatic brain injury in the United States: epidemiology and rehabilitation. Atlanta (GA): National Center for Injury Prevention and Control: Division of Unintentional Injury Prevention.
Cheng, F., Desai, R.J., Handy, D.E., Wang, R., Schneeweiss, S., Barabasi, A-L., Loscalzo, J. (2018). Network-based approach to prediction and population-based validation of in silico drug repurposing. Nat. Comm. 9, 2691.
Cohen TJ, Guo JL, Hurtado DE, Kwong LK, Mills IP, Trojanowski JQ, and Lee VM. (2011). The acetylation of tau inhibits its function and promotes pathological tau aggregation. Nat. Commun. 2: 252.
Cook, C., Carlomagno, Y., Gendron, T.F., Dunmoore, J., Scheffel, K., Stetler, C., Davis, M., Dickson, D., Jarpe, M., DeTure, M., Petrucelli, L. (2014). Acetylation of the KXGS motifs in tau is a critical determinant in modulation of tau aggregation and clearance. Hum. Mol. Genet. 23, 104-116.
Dutca, L.M., Stasheff, S.F., Hedberg-Buenz, A., Rudd, D.S., Batra, N., Blodi, F.R., Yorek, M.S., Yin, T., Shankar, M., Herlein, J.A., et al. (2014). Early detection of subclinical visual damage after blast-mediated TBI enables prevention of chronic visual deficit by treatments with P7C3-S243. Invest. Ophthalmol. Vis. Sci. 55, 8330-8341.
Evans, D.B., Rank, K.B., Bhattacharya, J., Thomsen, D.R., Gurney, M.E., and Sharma, S.K. (2000). Tau phosphorylation at serine 396 and serine 404 by human recombinant tau protein kinase II inhibits tau's ability to promote microtubule assembly. J. Biol. Chem. 275, 24977-24983.
Geisen, U., Benk, C., Beyersdorf, F., Klemm, R., Trummer, G., Ozbek, B., Kern, F., and Heilmann, C. (2015). Neuron-specific enolase correlates to laboratory markers of haemolysis in patients on long-term circulatory support. Eur J Cardiothorac Surg 48, 416-420.
Hara, M.R. and Snyder S.H. (2006). Nitric oxide-GAPDH-Siah: A novel cell death cascade. Cell. Mol. Neurobiol. 26, 527-538.
Irwin, D.J., Cohen, D.J., Grossman, M., Arnold, S.E., Xie, S.X., Lee, M.M-Y., and Trojanowski, J.Q. (2012). Acetylated tau, a novel pathological signature in Alzheimer's disease and other tauopathies. Brain 135, 807-818.
Isgro M.A., Bottoni, P., and Scatena, R. (2015). Neuron-specific enolase as a biomarker: biochemical and clinical aspects. Adv Exp Med Biol 867, 125-143.
Johnson, V.E., Stewart, W., and Smith, D.H. (2010). Traumatic brain injury and amyloid-b pathology: a link to Alzheimer's disease? Nat Rev Neurosci 11, 361-370.
Kornberg, M.D., Sen, N., Hara, M.R., Juluri, K.R., Nguyen, J.V.K., Snowman, A.M., Law, L., Hester, L.D., and Snyder S.H. (2010). GAPDH mediates nitrosylation of nuclear proteins. Nat. Cell Biol. 12, 1094-1100.
Li, Y., Li, Y., Li, X., Zhang, S., Zhao, J., Zhu, X., Tian, G. (2017). Head injury as a risk factor for dementia and Alzheimer's disease: a systematic review and meta-analysis of 32 observational studies. PLOS One 12, e0169650 .
Lubke, U., Six, J., Villanova, M., Boons, J., Vandermeeren, M., Ceuterick, C., Cras, P., and Martin, J.J. (1994). Microtubule-associated protein tau epitopes are present in fiber lesions in diverse muscle disorders. Am. J. Pathol. 145, 175-188.
Lucke-Wold, B., Seidel, K., Udo, R., Omalu, B., Ornstein, M., Nolan, R., Rosen, C., and Ross, J. (2017). Role of tau acetylation in Alzheimer's disease and chronic traumatic encephalopathy: the way forward for successful treatment. J. Neurol. Neurosurg. 4, 140.
Ma, V. Y., Chan, L., and Carruthers, K. J. (2014). The incidence, prevalence, costs, and impact on disability of common conditions requiring rehabilitation in the US: stroke, spinal cord injury, traumatic brain injury, multiple sclerosis, osteoarthritis, rheumatoid arthritis, limb loss, and back pain. Arch. Phys. Med. Rehabil. 95, 986-995.
Merck & Co, Inc. Whitehouse Station, MJ 08889, USA. Manufacturer data sheet, 9676203, Dolobid (Diflunisal).
Min, S-W., Cho, S-H., Zhou, Y., Schroeder, S., Haroutunian, V., Seeley, W.W., Huang, E.J., Shen, Y., Masliah, E., Mukherjee, C., et al. (2010). Acetylation of tau inhibits its degradation and contributes to tauopathy. Neuron 67, 953-966.
Mondello, S., Sorinola, A., Czeiter, E., Vamos, Z., Amrein, K., Synnot, A., Donoghue, E., Sandor, J., Wang, K.K.W., Diaz-Arrastia, R., et al. (2018). Blood-based protein biomarkers for the management of traumatic brain injuries in adults presenting to emergency departments with mild brain injury: a living systematic review and meta-analysis. J Neurotrauma July 2 doi: 10.1089/neu.2017.5182. Online ahead of print.
Nakamura, T. and Lipton, S. A. (2020). Nitric oxide-dependent protein post-translational modifications impair mitochondrial function and metabolism to contribute to neurodegenerative diseases. Antioxid. Redox. Signal. 32, 817-833.
Neddens, J., Temmel, M., Flunkert, S., Kerschbaumer, B., Hoeller, C., Loeffler, T., Niederkofler, V., Daum, G., Attems, J., Hutter-Paier, B. (2018). Phosphorylation of different tau sites during progression of Alzheimer's disease. Acta Neuropathologica Communications, 6, 52.
Okamura Y, Kawakami I, Watanabe K, Oshima J, Niizato K, Ikeda K, Akiyama H, and Hasegawa M. (2019). Tau progression in single severe frontal traumatic brain injury in human brains. J. Neurol Sci. 407: 116495.
Olsson, B., Zetterberg, H., Hampel, H., and Blennow, K. (2011). Biomarker-based dissection of neurodegenerative diseases. Prog Neurobiol 4, 520-534.
Oresic, M., Posti, J.P., Kamstrup-Nielsen, M.H., Takala, R.S.K., Lingsma, H.F., Mattila, I., Jantti, S., Katila, A.J., Carpenter, K.L.H., Ala-Seppala, H., et al. (2016). Human serum metabolites associate with severity and patient outcomes in traumatic brain injury. EBioMedicine 12, 188-126.
Perea JR, Lopez E, Diez-Ballesteros JC, Avila J, Hernandez F, and Bolos M. (2019). Extracellular monomeric tau is internalized by astrocytes. Front. Neurosci. 13: 442.
Pieper, A.A., Xie, S., Capota, E., Estill, S., Zhong, J., Long, J.M., Becker, G.L., Huntington, P., Goldman, S.E., Shen, C-H., et al. (2010). Discovery of a proneurogenic, neuroprotective chemical. Cell 142, 39-51.
Pieper, A.A. and Mcknight, S. L. (2018). Benefits of enhancing nicotinamide adenine dinucleotide levels in damaged or diseased nerve cells. Cold Spring Harb. Symp. Quant. Biol. 83, 207-217.
Ryan, J., Storey, E., Murray, A.M., Woods, R.L., Wolfe, R., Reid, C.M., Nelson, M.R., Chong, T.T.J., Williamson, J.D., Ward, S.A., Lockery, J.E., Orchard, S.G., Trevaks, R., Kirpach, B., Newman, A.B., Ernst, M.E., McNeil, J.J., Shah, R.C., ASPREE Investigator Group. (2020). Randomized placebo-controlled trial of the effects of aspirin on dementia and cognitive decline. Neurology 95, e320-e331.
Schafer, D.P., Jha, S., Liu, F., Akella, T., Mccullough, L.D., and Rasband, M.N. (2009) Disruption of the axon initial segment cytoskeleton is a new mechanism for neuronal injury. J. Neurosci. 29, 13242-13254.
Sen, T., Saha, P., and Sen, N. (2018). Nitrosylation of GAPDH augments pathological tau acetylation upon exposure to amyloid-β. Sci. Signal. 11, eaao6765.
Shahim, P., Politis, A, van der Merwe, A., Moore, B., Ekanayake, V., Lippa, S.M., Chou, Y-Y., Pha, D.L., Butman, J.A., Diaz-Arrastia, R., et al. (2020). Time course and diagnostic utility of Nfl, tau, GFAP, and UCH-LI in subacute and chronic TBI. Neurology 95, e623-e636.
Shin M-K, Vázquez-Rosa E, Cintrón-Pérez C, Riegel A, Harper MM, Ritzel D, Pieper AA. (2021). Characterization of the jet-flow overpressure model of traumatic brain injury in mice. Neurotrauma Reports (in press).
Shirakawa, K., Wang, L., Man, N., Maksimoska, J., Sorum, A.W., Lim, H.W., Lee, I.S., Shimazu, T., Newman, J.C., Schroder, S., et al. (2016). Salicylate, diflunisal, and their metabolites inhibit CBP/p300 and exhibit anticancer activity. eLife 5, e11156.
Sohn, P.D., Tracy, T.E., Son, H-I., Zhou, Y., Leite, R.E.P., Miller, B.L., Seeley, W.W., Grinberg, L.T., and Gan, L. (2016). Acetylated tau destabilizes the cytoskeleton in the axon initial segment and is mislocalized to the somatodendritic compartment. Mol. Neurodegen. 11, 47.
Tolan, N.V., Vidal-Folch, N., Algeciras-Schimnich, A., Singh, R.J., and Grebe, S.K. (2013). Individualized correction of neuron-specific enolase (NSE) measurement in hemolyzed serum samples. Clin Chim Acra 424, 216-221.
Tracy, T.E., Sohn, P.D., Minami, S.S., Wang, C., Min, S-W., Li, Y., Zhou, Y., Le, D., Lo, I., Ponnusamy, R.,et al. (2016). Acetylated tau obstructs KIBRA-mediated signaling in synaptic plasticity and promotes tauopathy-related memory loss. Neuron 90, 245-260.
Uehara, T., Nakamura, T., Yao, D., Shi, Z-Q., Gu, Z., Ma, Y., Masliah, E., Nomura, Y., and Lipton, S.A. (2006). S-nitrosylated protein-disulphide isomerase links protein misfolding to neurodegeneration. Nature 441, 513-517.
Vázquez-Rosa, E., Watson, M.R., Sahn, J.J., Hodges, T.R., Schroeder, R.E., Cintrón-Pérez, C.J., Shin, M.K., Yin, T.C., Emery, J.L., Martin, S.F., et al. (2019). Neuroprotective efficacy of a sigma 2 receptor/TMEM97 modulator (DKR-1677) after traumatic brain injury. ACD Chem. Neurosci. 10, 1595-1602.
Vázquez-Rosa E, Shin M-K, Dhar M, Chaubey K, Cintrón-Pérez C, Tang X, Liao X, Miller E, Barker S, Franke K, Koh Y, Crosby DR, Schroeder R, Emery J, Yin TC, Fujioka H, Reynolds JD, Harper MM, Jain MK, Pieper AA. (2020). P7C3-A20 treatment one year after TBI in mice repairs the blood-brain barrier, arrests chronic neurodegeneration, and restores cognition. Proceedings of the National Academy of Sciences USA 117: 27667-27675.
Vermersch, P., Sergeant, N., Ruchoux, M.M., Hofmann-Radvanyi, H., Wattez, A., Petit, H., Dwailly, P., and Delacourte, A. (1996). Specific tau variants in the brains of patients with myotonic dystrophy. Neurology 47, 711-717.
Waldmeier, P.C., Spooren, W.P., and Hengerer, B. (2000). CGP3466 protects dopaminergic neurons in lesion models of Parkinson's disease. Naunyn-Schmiedeberg's Arch. Pharmacol. 362, 526-537.
Wang, G., Han, T., Nijhawan, D., Theodoropoulos, P., Naidoo, J., Yadavalli, S., Mirzaei, H., Pieper, A.A., Ready, J.M., McKnight, S.L. (2014). P7C3 neuroprotective chemicals function by activating the rate-limiting enzyme in NAD salvage. Cell 58, 1324-1334.
Wattiez A-S, Castonguay WC, Gaul OJ, Waite JS, Schmidt CM, Reis A, Rea BJ, Sowers LP, Cintrón-Pérez C, Vázquez-Rosa E, Pieper AA, Russo AF. (2020). Different forms of traumatic brain injury cause different tactile hypersensitivity profiles. Pain (in press).
Wesseling H, Mair W, Kumar M. Schlaffner CN, Tang S, Beerepoot P, Fatou B, Guise AJ, Cheng L, Takeda S, Muntel J, Rotunno MS, Dujardin S, Davies P, Kosik KS, Miller BL, Berretta S, Hedreen JC, Grinberg LT, Seeley WW, Hyman BT, Steen H, and Steen JA. (2020). Tau PTM profiles identify patient heterogeneity and stages of Alzheimer's disease. Cell 183: 1699-1713.
Yang W-J, Chen W, Chen L, Guo Y-J, Zeng J-S, Li G-Y, and Tong W-S. (2016). Involvement of tau phosphorylation in traumatic brain injury patients. Acta Neurologica Scandinavica 135: 622-627.
Yin, T.C., Voorhees, J.R., Genova, R.M., Davis, K.C., Madison, A.M., Britt, J.K., Cintrón-Pérez, C.J., McDaniel, L., Harper, M.M., and Pieper, A.A. (2016). Acute axonal degeneration drives development of cognitive, motor, and visual deficits after blast-mediated traumatic brain injury in mice. eNeuro 10.1523/ENEURO.0220-16.2016.
Yin, T.C., Britt, J.K., De Jesús-Cortés, H, Lu, Y., Genova, R.M., Khan, M.Z., Voorhees, J.R., Shao, J., Katzman, A.C., Huntington, P.J. et al. (2014). P7C3 neuroprotective chemicals block axonal degeneration and preserve function after traumatic brain injury. Cell Rep. 8, 1731-1740.
Zetterberg, H. and Blennow, K. (2016). Fluid biomarkers for mild traumatic brain injury and related conditions. Nat Rev Neurol 12: 53-574.

### Example 2: Materials and Methods

Animals: For traumatic brain injury experiments with our standard model incorporating aspects of concussion, acceleration/deceleration, and blast wave exposure, 8-week-old male and female C57BL/6J (The Jackson Laboratory) mice, and male Wld^{S} mice, were used. All mice were maintained under temperature (22° C.-23° C.), light (12-hour light cycle from 6 AM to 6 PM), and humidity-controlled (40%-60%) conditions with free access to food and water. All animal work was approved by Louis Stokes Cleveland VA Medical Center Institutional Animal Care and Use Committee (animal protocol #18-050-MS-18-015).

Drug preparation and administration: CGP3466B (Sigma-Aldrich, SML1941) was dissolved in DMSO and then diluted in sterile saline. The final working stock was 0.0014 mg/ml for administering the 0.014 mg/kg dose. Intraperitoneal administration of CGP3466B was initiated 15 min or 24 h after injury, and tissues were harvested 6 h (for SNO-GAPDH, SNO-SIRT1 measurement) or 2 weeks (for ac-tau measurement) later, respectively. Salsalate (AdipoGen, AG-CR1-3574) was dissolved in DMSO and then diluted in sterile PBS. The final working stocks were as follows: 2.5 mg/ml (25 mg/kg dose), 1 mg/ml (10 mg/kg dose), 0.5 mg/ml (5 mg/kg dose). Daily intraperitoneal administration of salsalate was initiated 24 hr after injury and continued throughout behavioral testing. P7C3-A20 and FK866 (Sigma-Aldrich, F8557) were first dissolved in 1 vol of DMSO, followed by addition of 4 vol of Kolliphor and vigorously vortexing. The solution was then diluted with 30 vol of filtered 5% dextrose (pH 7.0). EX527 (Selleckchem, S1541) was dissolved in 1% DMSO+30% polyethylene glycol+1% Tween 80. Daily intraperitoneal administration of P7C3-A20 was initiated 24 hr after poly-traumatic brain injury. EX527 (10 mg/kg) was administer once a day and FK866 was treated twice per day, with the first injection given at the same time as P7C3-A20 (20 mg/kg) and the second injection given 6 h later (LoCoco et al., 2017 eLife). Diflunisal was first dissolved in 1 vol of DMSO, followed by addition of 2 vol of Kolliphor and vigorously vortexing. The solution was then diluted with 7 vol of saline to the appropriate concentration for administration at 25, 50, or 100 mg/kg.

### In Vivo Traumatic Brain Injury Models

Poly-traumatic brain injury: Eight-week old male, female C57BL/6J and male Wld^{S} mice were anesthetized with ketamine (100 mg/kg) and xylazine (10 mg/kg) via intraperitoneal (IP) injection and placed in an enclosed chamber constructed from an air tank partitioned into two sides and separated by a port covered by a mylar membrane. The pressure in the side not containing the mouse was increased to cause membrane rupture at 20 pounds per square inch (PSI), which generates an about 1-2 ms jet airflow of 137.9+/−13.79 kPa that passes through the animal's head. The head remains untethered in a padded holder, while the body is fully shielded by a metal tube. The jet of air produced upon membrane rupture provides a concussive injury, which is followed by acceleration/deceleration of the head and then exposure to the ensuing blast wave within an enclosed space.

Metabolomics: Ten 8-week old male mice were subjected to poly-traumatic brain injury as described above and ten were exposed to a sham-injury. Seven days post-injury, blood was collected retro-orbitally in K2-EDTA blood collection tubes. Plasma was separated from these blood samples and flash frozen in liquid nitrogen. Plasma samples were sent to Metabolon Inc, (Durham, North Carolina, USA) for Global Metabolomics Profiling using LC-MS. 245 biochemicals identified in these plasma samples were significantly different between TBI and Sham groups.

Controlled Cortical Impact (CCI) injury: Adult mice, 2 to 4 months of age, were anesthetized with ketamine (100 mg/kg) and xylazine (10 mg/kg) via intraperitoneal (IP) injection and placed on a heating pad to maintain body temperature. The animal's head was shaved and then placed on a stereotaxic frame where an incision was made through the skin, exposing the skull. For CCI injured mice, a ˜5 mm diameter craniectomy was made over the right parietal cortex (bregma: −2.0 mm; lateral −2.5 mm), leaving the dura intact. Mice were then subjected to a moderate CCI injury with a piston velocity of 4.0 m/s and depth of 0.55 mm using an eCCI-6.0 device (Custom Design & Fabrication, Virginia Commonwealth, VA, United States). Sham controls underwent an identical surgical procedure with the absence of the craniectomy and injury. The incision was closed using 4-0 nylon non-absorbable sutures (Ethicon, Inc., Piscataway, NJ, United States), and mice were placed in a clean, single housed cage on a heating pad. For hydration and analgesia, animals were administered 1 mL of lactated ringer and 1.0 mg/mL buprenorphine. Ipsilateral CCI injured and sham cortices were harvested at 6 hrs as well as 1, 3, 7, 21, and 42 days post-injury. At the desired time points, mice were anesthetized with a ketamine/xylazine cocktail and euthanized by cervical dislocation, followed by dissection and freezing of injured and sham cortices.

Acoustic Blast Overpressure (ABO) model: We employed the shock tube device as described (Newman et al., 2015; Allen et al., 2018). The exit port of the shock tube is sealed by a thin brass foil diaphragm. With the diaphragm in place, the chamber behind the diaphragm is charged with compressed air, to a back pressure of 80 psi. A pressure sensor and air pressure modulator controlled by commercial hardware and custom software are used to charge the cylinder to the desired pressure. A computer-controlled (MatLab) solenoid-driven hunting arrow mounted inside the chamber is used to pierce the diaphragm, once the criterion pressure is achieved; this triggers a blast with precise timing and sound pressure, which can be monitored by a pressure transducer probe placed at the outlet end of the shock tube and recorded by a commercial analog-to-digital converter and stored on a computer for later analysis using custom software. A wire mesh insert near the outlet end catches metal foil fragments (shrapnel), preventing penetrating injury to animals. Rats (adult male Long-Evans rats) were deeply anesthetized (see Animals section) and placed into a custom-built metal holder, with body axis at right angles to the shock tube. Only the head was exposed (typically the left side), approximately 2.5 inches from the outlet end of the shock tube. A dual-blast paradigm, allowing a 1-month interval between blast exposures, each with a sound pressure of 63 kPa (190 dB-SPL) was employed. Animals were closely monitored at regular intervals for viability and any signs of distress following blast exposure.

In vitro traumatic brain injury model: SH-SY5Y cells were grown in DMEM:F12 (1:1)(Gibco, 11320-033) containing 10% fetal bovine serum (Gibco, 26140-079) and 1% penicillin/streptomycin (Gibco, 15140-122). Cells were seeded at 2×10⁶ cells per well on the collagen I precoated 6-well flexible-bottomed culture plates (Flexcell International Corporation, BF-3001C). Flag-TauWT, Flag-TauKQ, Flag-TauKR (Gan laboratory) plasmids were transfected with FuGene® HD transfection reagent (Promega, E2311) according to the manufacturer's instructions. Six hours after transfection, cells were injured by 90-ms bust of pressurized medicinal air using the Cell Injury Controller II system (Custom Design & Fabrication Inc., Sandston, VA). Cells were harvested two hours after injury. LDH release was measured using a commercial assay kit (Cytotoxicity Detection Kit^{Plus} (LDH), Roche, 04-744-926-001) according to the manufacturer's instructions.

Western blotting: Western blotting was performed as described previously (Min et al., 2010), with the 9AB antibody against acetylated-tau generated in the Gan laboratory. Briefly, cortical and hippocampal tissues were sonicated in RIPA buffer (Sigma-Aldrich, R0278) containing protease and phosphatase inhibitor cocktail (Thermo Scientific, #1861284), 1 mM phenylmethyl sulfonyl fluoride (Sigma Aldrich, P7626), and histone deacetylase inhibitors such as 5 mM nicotinamide (Sigma-Aldrich, 72340) and 1 μM trichostatin A (Sigma-Aldrich, T8552). Lysates were centrifuged at 170,000 g at 4° C. for 15 min and at 18,000 g at 4° C. for 10 min, after which protein concentrations of supernatants were measured by bicinchoninic acid assay (Thermo Scientific, A53225). Proteins were heated in a Laemmli Sample Buffer (Bio-Rad Laboratories, Inc., #1610737) with beta-mercaptoethanol (Bio-Rad Laboratories, Inc., #1610710) for 5 min, and then resolved in 4-20% Criterion TGX Stain-Free gels (Bio-Rad Laboratories, Inc., #5678095). Stain-free gels were exposed to ultra-violet light by ChemiDoc™ MP Imaging system (Bio-Rad Laboratories, Inc.) to visualize total plasma proteins. Proteins were transferred onto 0.2 μm polyvinylidene fluoride membranes (Bio-Rad Laboratories, Inc., #1704157) with the Trans-Blot Turbo system (Bio-Rad Laboratories, Inc.). Membranes were blocked with 5% nonfat dry milk in tris-buffered saline-tween 20 (TBST) for 1 h at room temperature, and then incubated with primary antibodies at 4° C. overnight. The following antibodies were used to probe target proteins: rabbit anti-ac-tau (Li Gan laboratory, 1:500), mouse anti-tau (abcam, ab80579, 1:5000), mouse anti-tau (ThermoFisher Scientific, #13-6400, 1:5000), mouse anti-β-actin (Santa Cruz Biotechnology, sc-47778, 1:1000), mouse anti-GAPDH (EMD Millipore Corp, MAB 374, 1:5000), mouse anti-NeuN (EMD Millipore Corp, MAB 377, 1:1000), mouse anti-GFAP (Thermo Scientific, MA5-12023, 1:1000), mouse anti-SIRT1 (Abcam, ab110304, 1:500), rabbit anti-ac-tau (AnaSpec, AS-56077, 1:250), rabbit anti-p300 (Santa Cruz Biotechnology, sc-584, 1:500), rabbit anti-CBP (Cell Signaling Technology, #7389, 1:1000), rabbit anti-acetyl K5 histone 2A (Abcam, ab1764, 1:1000), rabbit anti-histone 2A (Cell Signaling Technology, #12349, 1:1000), rabbit anti-acetyl K18 histone 3 (Abcam, ab1191, 1:10,000), rabbit anti-histone 3 (Cell Signaling Technology, #4499, 1:1000), rabbit anti-phospho-tau (Ser 202) (Cell Signaling Technology, #11834, 1:1000), rabbit anti-phospho-tau (Ser 262) (ThermoFisher Scientific, 44-750G, 1:1000), mouse anti-phospho-tau (Ser 396, 404) (Gift from Peter Davies, 1:1000), rabbit anti-KIBRA (Santa Cruz Biotechnology, sc-133374, 1:500), rabbit anti-PSD95 (Cell Signaling Technology, #2507, 1:1000), mouse anti-AnkG (Santa Cruz Biotechnology, sc-12719, 1:1000), rabbit anti-βIV spectrin (Rasband laboratory, 1:500), rabbit anti-TNF-alpha (Cell Signaling Technology, #11948, 1:2000), rabbit anti-ac-p300/CBP (Cell Signaling Technology, #4771, 1:1000), rabbit anti-tau (Cell Signaling Technology, #46687, 1:1000), rabbit anti-GFAP (Invitrogen, PA5-16291, 1:1000), rabbit anti-Flag (Cell Signaling Technology, #2368, 1:1000), rabbit anti-cleaved caspase 3 (Cell Signaling Technology, #9661, 1:500). After primary antibody incubation, membranes were rinsed with TBST (3×5 min) and subsequently incubated with horseradish peroxidase conjugated-secondary antibodies. SuperSignal™ West Femto Maximum Sensitivity Substrate (Thermo Scientific, #34096) was used to detect band by BioSpectrum 810 Imaging System (UVP, Upland, CA). Densitometry quantification of western blot signas was conducted by ImageJ version 1.42 software (National Institutes of Health, Bethesda, MD).

Neuron and non-neuron isolation: Three cortices were pooled and collected in DPBS with Calcium, Magnesium, Glucose and Pyruvate (Thermo Fisher Scientific, 14287080) and processed using the MACS Adult Brain dissociation kit (MACS, 130-107-677) to generate a single cell suspension. Briefly, each sample was digested using a combination of enzymatic and mechanical dissociation. For enzymatic dissociation, Enzyme mixes 1 and 2 (provided in the kit) were used for mechanical dissociation. Samples immersed in the enzymes mixes were placed in MACS C-tubes, which were placed in the MACS Octo dissociator with heaters for 30 min at 37° C. The dissociated samples were further processed for debris removal by first passing them through a MACS 70 μm smart strainer and then using MACS Debris Removal reagent. Lastly, RBC lysis was performed to achieve the final single cell suspension of brain cells. This single cell suspension was further processed using the MACS Neuronal isolation kit (MACS, 130-115-389) to separate the single cell suspension into neuronal and non-neuronal populations. Briefly, cells were mixed with MACS Non-neuronal cell Biotin antibody cocktail for 5 min. After washing with DPBS (with 0.5% FBS), cells were incubated with Anti-Biotin Microbeads. After a 10-min incubation, cells were passed through a column attached to a magnetic stand, which results in binding of non-neuronal cells (labelled by microbead-biotin antibodies) to the column, and the flow-though was collected as neuronal population. The non-neuronal population was collected by removing the column from the magnetic stand and placing the plunger in the column to flush out the non-neuronal cells.

SNO-resin assisted capture (SNO-RAC): SNO-RAC was performed as described previously (Forrester, 2009). Mouse cerebral cortex was mechanically homogenized in lysis buffer containing 100 mM HEPES/1 mM EDTA/0.1 mM neocuproine (HEN), 150 mM NaCl, 0.1% (vol/vol) Nonidet P-40 (NP-40), 0.2% S-methylmethanethiosulfonate (MMTS) and protease and phosphatase inhibitor cocktail (Thermo Scientific, #1861284). After two times centrifugation (20,000 g at 4° C. for 20 min), protein concentration of supernatants was determined using Coomassie protein assay (Thermo Scientific, #1856210). Total lysates were treated with 0.2% MMTS and 2.5% SDS, and then incubated at 50° C. for 20 min. Proteins were precipitated with pre-chilled (−20° C.) acetone and centrifuged at 4,255 g at 4° C. for 12 min. After washing pellets with 70% acetone three times, proteins were sonicated in HEN buffer containing 1% SDS. Precipitation of proteins was repeated with −20° C. acetone, and the final pellets were resuspended in HEN/1% SDS. Proteins were incubated with freshly prepared 30 mM ascorbate and 50 μl of thiopropyl sepharose (GE Lifesciences, 17-0420-01, Pittsburgh, PA) and rotated in the dark for 3 h. After centrifugation at 1,200 g for 30 sec, the bound SNO proteins were sequentially washed three times with HEN/1% SDS and two times with 1/10 diluted HEN/1% SDS. SNO-proteins were then eluted with 2× Laemmli Sample Buffer (Bio-Rad Laboratories, Inc., #1610737) with 10% beta-mercaptoethanol (Bio-Rad Laboratories, Inc., #1610710) and analyzed by SDS-PAGE and immunoblotting.

Postsynaptic density fractionation: To enrich the postsynaptic density (PSD), synaptosomal membranes were isolated from adult mice following Kristian et al 2010 with minor modifications (Kristian, 2010; Cao et al., 2007). Brains were extracted, and the cortex and hippocampus were quickly dissected. Tissue samples were immediately homogenized with 8 passes of a Teflon on glass Potter-Elvehjem homogenizer in subcellular isolation buffer (SIB: 225 mM mannitol, 75 mM sucrose, 2 mM K₂HPO₄, 5 mM HEPES, 1 mM EGTA, 0.1% fatty acid free bovine serum albumin). Differential centrifugation was performed at 1,500 g followed by 21,000 g on the supernatant to sequentially de-enrich unlysed cells and cytosolic and endoplasmic reticulum proteins, respectively. The resulting membrane-enriched pellet was separated at 21,000 g for 8 minutes over a discontinuous Percoll gradient comprised of 15%, 24%, and 40% Percoll steps. Synaptosomes were collected between the 15% and 24% Percoll interfaces. Synaptosomes diluted in SIB were pelleted at 10,000 g. All steps were performed on ice with centrifuging at 4° C.

Immunohistochemistry: Mice were transcardially perfused with cold 1× phosphate-buffered saline (PBS) followed by 4% paraformaldehyde in PBS at pH 7.4 under anesthesia. Brains were carefully removed and post-fixed in 4% paraformaldehyde overnight at 4° C. Brains were immersed in 30% sucrose in PBS for 72 h at 4° C. and then rapidly frozen in 2-methylbutane pre-cooled to −20° C. with dry ice. Brains were cut coronally (40 μm) and sections were stored in cryoprotective solution (150 mM Ethylene glycol, 100 mM glycerol, 250 mM PBS) at −20° C. For tau and NeuN staining, sections were washed three times with PBS for 5 min and then treated with 0.2% Triton X-100 in PBS for 15 min. Sections were washed with PBS and then incubated with 100 mM glycine for 15 min. After blocking sections using blocking buffer (1% bovine serum albumin, 10% normal goat serum, 0.3 M glycine) for 30 min, primary antibodies (Tau, Invitrogen, #13-6400, 1:100, NeuN, EMD Millipore Cor., #MABN-140, 1:500) were incubated overnight at 4° C. Sections were washed three times with PBS (5 min each) and then incubated with Alexa Fluor 488 goat anti-mouse (Invitrogen, A11001, 1:200) or Alexa Fluor 568 goat anti-rabbit (Life technologies, A11011, 1:200) at room temperature for 2 h. Sections were mounted on slides and then coverslipped with Prolong diamond antifade mountant (Invitrogen, P36961). AnkG and βIV spectrin staining were performed as described previously (Peter et al., 2016). Sections were permeabilized with 0.3% Triton X-100 and blocked with 10% normal goat serum at room temperature for 1 h. Sections were incubated with primary antibodies (AnkG, NeuroMab, N106/36, 1:500, βIV spectrin (Rasband laboratory, 1:500) overnight at 4° C. and then with Alexa Fluor 488 goat anti-mouse (Invitrogen, A11001, 1:300) or Alexa Fluor 568 goat anti-rabbit (Life technologies, A11011, 1:300) at room temperature for 1 h. For silver staining, sections were collected in 0.1 M phosphate buffer (pH 7.4) containing 4% paraformaldehyde and fixed for 7 days at 4° C. Sections were then processed for the detection of axon degeneration with FD NeuroSilver Kit II (FD Neurotechnologies, PK301) according to the manufacturer's instructions. Sections were subsequently mounted on slides, cleared in xylene, and coverslipped with Permount (Fisher Scientific, Fair Lawn, NJ).

Immunohistochemistry for synaptic vesicle 2 protein: Mice were transcardially perfused with PBS. The brain was removed and placed in 4% paraformaldehyde (PFA) for 48 h, followed by 30% sucrose for 48 h at 4° C. A freezing microtome (Leica) was used to make 30-μm-thick brain sections. The brain sections were first permeabilized in blocking solution containing PBS with 0.5% Triton-X100 and 10% normal donkey serum for 1 hour at room temperature. Then they were incubated overnight with an SV2 antibody (Developmental Studies Hybridoma Bank) in blocking solution followed the next day by a 1 hour incubation with an Alexa-conjugated secondary antibody (Life Technologies) at room temperature. Confocal images were acquired on a CSU22 spinning disk confocal system (Yokogawa) with a Ti-E microscope (Nikon) using a 60× oil immersion objective lens. The quantification of immunofluorescence was performed using ImageJ software (NIH).

Quantification of immunohistochemistry: All images were acquired with Zeiss LSM710 confocal microscope and Zeiss Axiolmager.M2 with monochromatic digital camera (Zeiss AxioCam MRm Rev. 3). To visualize the axon initial segment, twenty serial optical sections (0.5 um steps) were projected into a single image. The microscope light intensity and exposure time were kept constant for silver stained images. ImageJ version 1.42 software (National Institutes of Health, Bethesda, MD) was used to analyze the intensity of the AIS and integrated density of silver staining with the plugin of the color deconvolution method as previously described (Ruifrok and Johnston, 2001). The operator performing quantification was blinded to condition and treatment.

Quantitative real-time PCR: Total RNA was extracted from frozen cortex using High Pure RNA Isolation Kit (Roche Life Science, USA) according to the manufacturer's protocol. RNA concentrations were determined by UV visible absorption spectra, using Nanodrop 2000 (Thermo Scientific, USA). First-strand cDNA was synthesized from total RNA (500 ng) using iScript cDNA Synthesis Kit (Bio-Rad Laboratories Inc., 1708891, USA) according to the manufacturer's instruction. Quantitative PCR was performed in triplicate using Fast SYBR Green Master Mix on a Step One Plus Real-time PCR System (Applied Biosystems, USA). Following primers (5′ to 3′) were used to examine the gene expression of CCL5, ILIβ, CCL2, and GAPDH; CCL5 (F): GGG TAC CAT GAA GAT CTC TGC (SEQ ID NO.: 7), (R): GCG AGG GAG AGG TAG GCA AAG (SEQ ID NO.: 8), IL-1B (F): GAG CAC CTT CTT TTC CTT CAT CTT (SEQ ID NO.: 9), (R): CAC ACA CCA GCA GGT TAT CAT CA (SEQ ID NO.: 10), CCL2 (F): GGC TCA GCC AGA TGC AGT TAA (SEQ ID NO.: 11), (R): CCT ACT CAT TGG GAT CAT CTT GCT (SEQ ID NO.: 12), GAPDH (F): TGT GTC CGT CGT GGA TCT GA (SEQ ID NO.: 13), (R): CCT GCT TCA CCA CCT TCT TGA (SEQ ID NO.: 14). Fold change of gene expression was calculated by comparative CT quantification method (Schmittgen and Livak, 2008) and normalized to the expression of GAPDH.

Plasma sampling and albumin/immunoglobulin depletion: Mice were anesthetized with ketamine (100 mg/kg) and xylazine (10 mg/kg) via intraperitoneal (IP) injection and blood samples were collected in EDTA tubes (Becton, Dickinson and Company, 365974) by retro-orbital bleeding and then plasma was separated at 2,000 g at 4° C. for 15 min. Albumin and immunoglobulin depletion were performed according to the manufacturer's instructions (Bio-Rad Laboratories, Inc., 732-6701). Briefly, Aurum serum protein columns were washed two times with 1 ml of Aurum serum protein binding buffer and centrifuged at 10,000 g for 20 sec. Sixty microliter of human and mouse plasma sample was mixed with 180 μl of Aurum serum protein binding buffer and 200 μl of the diluted plasma sample were added to the top of the resin bed. Column was gently vortexed every 5 min for a total incubation time of 15 min and then centrifuged at 10,000 g for 20 sec. Eluate was collected in collection tube and resin was washed with 200 μl of the binding buffer. After centrifugation (10,000 g, 20 sec), the eluate was collected in a previous collection tube.

NAD⁺ measurement: Cerebral cortex was dissected as quickly as possible on a cold metal block and flash frozen in liquid nitrogen. Samples were stored at −80° C. until assay. Tissue NAD⁺ determination was performed according to the manufacturer's instructions (BioVision, K337-100). Brain tissues were washed with cold PBS and homogenized in NADH/NAD extraction buffer and then centrifuged at 14,000 rpm at 4° C. for 15 min. Supernatants were filtered using 3 kDa molecular weight cutoff centrifugal filters (Merck Millipore Ltd., UFC500324) to remove enzymes that consume NADH and NAD. Fifty microliters of samples were transferred into a 96 well plate for total NADH and NAD, and 50 μl of sample was heated at 60° C. for 30 min to decompose NAD and then also added into a 96 well plate for measuring total NADH. Ten microliters of 1 nmol/μl NADH standard was diluted with 990 μl of NADH/NAD extraction buffer and then transferred into a 96 well plate to make 0, 20, 40, 60, 80, and 100 pmol/well. One hundred microliters of NAD cycling enzyme mix was added into standard, and samples were then incubated in the plate at room temperature for 5 min. After adding 10 μl of NADH developer, the plate was read at OD 450 nm.

Tau seed amplification assay (AD RT-QuIC): Twenty percentage w/v brain homogenates were prepared from hippocampus and cortex in ice-cold 1×PBS with protease inhibitors (Roche, complete, EDTA-free) using 1 mm zirconia/silica beads (Biospec Products) and a BeadMill 24 (Fisher Scientific). Brain homogenates were used to seed the AD RT-QuIC. Assay conditions used were as previously published (Kraus et al., 2019) with the addition that both heparin (USP, 1235820) and poly-L-glutamate (Sigma, P1818) were independently tested in evaluating seeding activity. Synthetic fibrils generated from recombinant tau encoding aa 306-378 were used as a positive control. For analysis of human TBI and control plasma samples (immunoglobulin depleted), 5 microliters was used to seed the reaction, with triplicate wells analyzed for each biological replicate. 18 mg of synthetic fibrils/5 microliters of control plasma was used as a positive control to verify that plasma matrices were not inhibitory to the RT-QuIC reactions.

Barnes maze: The Barnes maze apparatus consisted of a gray circular platform (91 cm in diameter and 90 cm in height), with 20 equally spaced holes 5 cm in diameter along the perimeter (Stoelting Co.). One of these holes contained a recessed escape chamber located under the platform. Four different and equally-spaced visual cues with different shapes and colors were hung on a black circular curtain surrounding the maze. The training session consisted of four trials per day for four consecutive days. In each trial, the mouse was gently released in the middle of the maze under the cylindrical chamber, and after 5 sec elapsed the covering chamber was lifted to allow the mouse to explore the maze. If a mouse failed to find the escape chamber within 60 sec, it was manually guided to the escape chamber and then allowed to stay in the chamber for 30 sec. Both the platform and the escape chamber were cleaned thoroughly between individual trials. On day 5, 24 h after the last training day, the escape chamber was removed and mice were allowed to explore the maze for 60 sec. Total and primary latency were measured during training days, and latency to first nose pokes in escape hole and time spent in target quadrant were measured for memory test on the probe trial day. Any-maze video tracking software (Stoelting Co.) was used to acquire measurements. Analysis was conducted blind to treatment group.

Foot slip test of motor function: Mice were trained to cross an 80 cm-long beam over two days and then tested on day 16. Video of the mice was recorded and analyzed by observers blind to treatment group.

Statistics: GraphPad Prism 7 software was used to perform all statistical analyses.

### Human Study

Study population: This is a retrospective study of plasma samples from subjects with TBI admitted to the neuroscience intensive care unit at the Memorial Herman Hospital-Texas Medical Center from December 2017 to April 2019. Inclusion criteria were age>18, presented after TBI (ACRM criteria: loss of consciousness, posttraumatic amnesia, alteration of consciousness), underwent a brain CT, fluency in English or Spanish, ability to provide consent (or consent obtainable from surrogate), visual acuity/hearing adequate for testing and neurologically intact prior to injury. Exclusion criteria were patients with past medical history (including bipolar disorder, seizures, dementia, depression, schizophrenia, HIV, cancer (current treatment that would interfere with follow-up), end-stage renal disease (on dialysis), severe polytrauma that would interfere with follow-up, modified Rankin scale (mRS)>1 (i.e. uses walker or need assistance with daily actives), claustrophobia, lives greater than 2 hour from hospital, low interest/low probability for follow-up, prisoner, pregnant women, penetrating TBI, current participation in interventional trial and risk of imminent death.

TBI sample collection and storage: Blood samples were collected at 5 pre-determined time-points: <24 hours of injury (T₁), during 24-48 hours of injury (T₃), during 3-5 days of injury (T₄), during 6-8 days of injury (T₅) and >10 days after injury (T₆).

Blood samples were collected at 5 time-points: <24 hours after TBI (T₁), 24-48 hours post-TBI (T₂), 3-5 days post-TBI (T₃), 6-8 days post-TBI (T₄) and >10 days post-TBI (T₅). We randomly selected 45 subjects, age- and gender-matched with 25 non-neurologically-impaired healthy subjects. Ninety TBI plasma samples were analyzed: 44 at T₁, 23 at T₂, 6 at T₃, 7 at T₄, and 10 at T₅. Since fewer samples were collected after T₁, results obtained from T₃ and T₄ (24-120 hours post-TBI), and from T₅ and T₆ (>120 hours post-TBI), were grouped for analysis. Mean participant age (±SD) was 50±18 years, and average age between TBI patients and healthy subjects was similar (48±20 vs 54+11, p>0.05; Table 1). Sex-ratio was equivalent across TBI and controls (89% vs 75% male, p>0.05).

TABLE 1
Study Subject demographics
Study Subject Demographics

| Controls (n = 24) | |
|---|---|
| Age (mean, SD) | 54(11.7) |
| Male, n (%) | 18(75) |
| TBI (n = 45) | |
| Age | 48(20.6) |
| Male, n (%) | 40(88) |
| TBI (by grade) | |
| Mild (n = 15) | |
| Age | 52(21) |
| Male, n (%) | 13(86) |
| Moderate (n = 15) | |
| Age | 44(21) |
| Male, n (%) | 15(100) |
| Severe (n = 15) | |
| Age | 48(19) |
| Male, n (%) | 12(80) |

SD: Standard Deviation

Due to myriad reasons, samples from all patients at all time-points are not available. Blood was drawn from existing lines or by venipuncture and collected into sterile vacutainers per time point. The samples were placed on ice immediately after collection and transported to the laboratory for centrifugation within an hour of draw (at 1460×g for 10 minutes at 4° C.), generating plasma. The plasma was centrifuged a second time (at 1460×g for 10 minutes at 4° C.) in order to generate platelet-poor plasma. Plasma was divided into aliquots and frozen at −80° C. until analysis.

TBI samples: Based on admission GCS (appendix 2), subjects were grouped by injury grade as mild (GCS between 13 and 15), moderate (GCS between 9 and 12) and severe (GCS less than 9). During the study period, 85 TBI subjects were consented (27 mild, 23 moderate, 34 severe and 1 unknown). From these subjects, we randomly selected 45 subjects: 15 mild TBI subjects 15 moderate TBI subjects and 15 from severe TBI so that subjects across injury severity grade (mild, moderate or severe) were matched for age. Additionally, all TBI subjects (n=45) were also matched for age and sex with respect to the control subjects. In total, 90 TBI samples were analyzed: 44 at T₁, 23 at T₃, 6 at T₄, 7 at T₅, and 10 at T₆. Since samples collected after the T₁ period were sparse, samples obtained from T₃ and T₄ (from 24-120 hours post-TBI), and, samples from T₅ and T₆ (>120 hours post-TBI). were grouped together for analysis. Control samples: Plasma samples from 25 non-neurological subjects were used as controls (patients were approached and enrolled at the UT Physician Cardiology clinic). Blood was drawn by venipuncture and collected into sterile vacutainers and immediately placed on ice. For processing of plasma, the tubes were centrifuged at 1460×g for 10 minutes at 4° C. followed by a second centrifugation at 1460×g for 10 minutes at 4° C. to generate platelet-poor plasma. Plasma was then aliquoted and stored at −80° C. until analysis. Age and sex were matched across the control cohort and TBI cohort.

Clinical and Demographic information: Demographic and clinical information including past medical history, age, sex, Glasgow coma scale (GCS) at admission.

Statistical analysis: Descriptive statistics were calculated for demographic variables in TBI and control cohorts (Table 1). To describe differences in age and sex, we used the Student's t-test, χ2-test, and Fisher's exact test. Statistical analyses were performed using open-source software packages in R (v3.1.3).

Standard Protocol Approvals, Registrations, and Patient Consents: The study was conducted with the approval of the institutional IRB (IRB Number HSC-MS-17-0776 (Molecular and Microbiome Mechanisms after Neurological Injury), HSC-MH-17-0452 (Biorepository of Neurological Disorders Registry and Tissue Repository at UT Health) and #EM-15-35 (University Hospitals Case Medical Center, Center for Clinical Research and Technology, OH). Written informed consent was obtained from the patient or surrogate.

Quanterix: Plasma Nfl, UCHL1, GFAP, pTau181 and Tau from control and TBI patient's samples were measured by using Simoa® Neurology 4-Plex B kit and Simoa® pTau-181 advantage V2 kit by Quanterix The Science of Precision Health (Billerica, MA).

Diflunisal pharmacokinetics: Diflunisal levels in mouse plasma and brain were monitored by LC-MS/MS using an AB Sciex (Framingham, MA) 4000 QTRAP® mass spectrometer coupled to a Shimadzu (Columbia, MD) Prominence LC. Diflunisal was detected with the mass spectrometer in negative MRM (multiple reaction monitoring) mode by following the precursor to fragment ion transitions 248.9 to 204.9 (quantifier ion) and 248.9 to 184.9 (qualifier ion). An Agilent C18 XDB column (5 micron, 50×4.6 mm) was used for chromatography for PK studies with the following conditions: Buffer A: dH20+0.1% formic acid, Buffer B: acetonitrile+0.1% formic acid, 0-1.0 min 5% B, 1.0-1.5 min gradient to 100% B, 1.5-3.0 min 100% B, 3.0-3.2 min gradient to 5% B, 3.2-4.5 5% B. Tolbutamide (transition 269.1 to 169.9) from Sigma (St. Louis, MO) was used as an internal standard (IS). Pharmacokinetic studies were performed by injecting 8 week old C57BL/6J male mice with diflunisal formulated in 10% DMSO, 20% Kolliphor EL, 70% saline. 60 or 180 minutes post-dose, animals were anesthetized with ketamine (100 mg/kg) and xylazine (10 mg/kg) via intraperitoneal (IP) injection. Blood was collected using the anticoagulant ACD, and then brains were harvested after transcardial perfusion with 1×PBS. Tissues were weighed before snap freezing and blood spun at 9600×g to collect plasma which was stored frozen along with brain tissue at −80° C. until analysis. Brain tissue was homogenized in a 3-fold volume (weight by volume) of PBS to generate a homogenate. 100 μl of plasma or tissue homogenate was mixed with 100 μl of acetonitrile plus 0.2% formic acid and 100 ng/ml N-benzylbenzamide IS. The samples were vortexed 15 sec, incubated at room temp for 10′ and spun twice at 16, 100×g 4° C. in a refrigerated microcentrifuge. Standard curves were generated using blank plasma (Bioreclamation, Westbury, NY) or blank tissue homogenate spiked with known concentrations of diflunisal and processed as described above. The concentration of drug in each time-point sample was quantified using Analyst software (Sciex). A value of 3-fold above the signal obtained from blank plasma or tissue homogenate was designated the limit of detection (LOD). The limit of quantitation (LOQ) was defined as the lowest concentration at which back calculation yielded a concentration within 20% of theoretical.

Diflunisal protein binding: Protein binding of diflunisal in mouse plasma or brain homogenate was determined by rapid equilibrium dialysis using RED chambers (Thermo Scientific, Waltham, MA). On the day of the RED experiment, frozen mouse plasma and 4× homogenized brain was thawed in a water bath at 37° C. Then it was equilibrated for 45 minutes at 37° C. in an atmosphere of 5% CO₂. The pH of PBS was confirmed within 7.4±0.1. The pH of plasma and brain was measured and adjusted to 7.4±0.1 using concentrated acid or base. Plasma and brain diluted at 1:20 (final) with PBS and used for all subsequent steps. An aliquot of plasma or brain was spiked with compound to a compound concentration of 5 μM and vortex mixed. Enough non-spiked matrix remained to enable matrix matching of dialysate at the end of the binding assessment. This matrix was stored at 37° C. in an atmosphere of 5% CO₂. For each matrix, dialysis was performed using n=4 individual RED units with 200 μL of compound spiked plasma at 5 μM in the donor chamber and 400 μL of PBS in the dialysate chamber. The plate containing the RED units was sealed with a gas-permeable seal and incubated at 37° C. for 6 hours under a 5% CO₂ atmosphere in an orbital shaker set to 100 rpm. At the end of the dialysis period, aliquots were taken from the donor and dialysate chambers of each RED unit to obtain post-dialysis measures of bound and unbound compound concentration. Donor, dialysate, and plasma or brain stability samples were analyzed by using a matrix matching approach whereby each sample was mixed in a 1:1 ratio with the opposite medium (blank matrix or PBS). The matrix matched samples were then crashed with 200 μL acetonitrile+formic acid (0.1% final)+tolbutamide IS (50 ng/mL final). Tubes were vortex mixed for 10 seconds and incubated at room temperature for 10 minutes. Then they were centrifuged at 16, 100×g for 5 minutes. Diflunisal levels in both chambers were measured by LC-MS/MS in comparison to a standard curve prepared in 1:1 matrix: PBS as described above. For each matrix, stability was assessed by maintaining individual aliquots of compound-spiked matrix at 37° C. and 5% CO₂ for 0 and 6 hours. At each time point, n=2 50 μL aliquots were matrix matched and crashed. Stability of diflunisal in plasma and brain was 99% over 6 hours as assessed by LC-MS/MS. Fraction unbound (fu) was determined based on the following equations:

$${f}_{u} = \frac{{C}_{dialysate}}{{C}_{donor}} \\ DF = \frac{Total volume of solution}{Volume of aliquot} \\ {f}_{u undiluted} = \frac{1 / DF}{( ( 1 / {f}_{u diluted} ) - 1 ) + 1 / DF} \\ {f}_{u} - fraction unbound \\ {C}_{dialysate} - concentration of compound in dialysate chamber after dialysis \\ {C}_{donor} - concentration of compound in donor chamber after dialysis \\ DF - dilution factor for plasma dilution \\ {f}_{u undiluted} - fraction unbound after correcting for plasma dilution \\ {f}_{u diluted} - fraction unbound of diluted plasma , \\ calculated using the {f}_{u} equation above if using diluted plasma$$

### Pharmacoepidemiologic Validation

Study cohorts. The IBM® MarketScan® Medicare Supplemental Database is one of the first in the U.S. to profile the healthcare experience of retirees with Medicare supplemental insurance paid by employers. The MarketScan Medicare Supplemental Database provides detailed cost, use and outcomes data for healthcare services performed in both inpatient and outpatient settings. For most of the population, the medical claims are linked to outpatient prescription drug claims and person-level enrollment data through the use of unique patient or enrollee identifiers. Beneficiaries in the MarketScan Medicare Supplemental Database have drug coverage; therefore, drug data are available and provide additional, often valuable, information. This feature makes the database a robust tool for pharmacoeconomic and outcomes research and helps provide insight into the drug use and spending patterns of older Americans. In this study, the pharmacoepidemiology study utilized the MarketScan Medicare Claims database from 2012 to 2017. The dataset included individual-level diagnosis codes, procedure codes and pharmacy claim data for 7.23 million patients. Pharmacy prescriptions of salsalate, diflunisal, and aspirin were identified by using RxNorm and National Drug Code (NDC).

Outcome measurement. For a subject, a drug episode is defined as from drug initiation to drug discontinuation. Specifically, drug initiation is defined as the first day of drug supply (i.e. 1^{st} prescription date). Drug discontinuation is defined as the last day of drug supply (i.e. last prescription date+days of supply) and without drug supply for the next 60 days. In another word, gaps of less than 60-day of drug supply were allowed within a drug episode. The drug cohort included the first drug episode for each subject. For the final cohorts, demographic variables including age, race, sex and geographical location were collected. Additionally, diagnoses of hypertension (HT), type 2 diabetes (T2D), and coronary artery disease (CAD) before drug initiation were collected (Table 2). These variables were specifically selected to address potential confounding biases. Last, a control cohort was selected from patients who were not exposed to salsalate and diflunisal. Specifically, control exposures were matched to the exposures (n strata=10) by initiation time, enrollment history, gender, HT diagnose, T2D diagnose and CAD diagnose. The outcome was time from drug initiation to diagnose of AD and TBI defined by ICD9/10 codes (Table 3). For drug cohorts, observations without diagnosis of AD were censored at the end of drug episodes. For the control cohort, the corresponding drug episode starting date was used as the starting time. Observations without diagnosis of AD were censored at the corresponding drug episode's end date. The detailed description are provided in a previous study (Cheng et al., 2018).

TABLE 2
Dataset for pharmacoepidemiologic analysis in Alzheimer's disease.

| | | | | Mean | Geographical location (%) | | | | | | | |
|---|---|---|---|---|---|---|---|---|---|---|---|---|
| | Sample | # of | Female | Age | | North | | | | CAD | T2D | HTN |
| Group | Size | AD | (%) | (SD) | Northeast | Central | South | West | NA | (%) | (%) | (%) |

Aspirin

| | | | | | | | | | | | | |
|---|---|---|---|---|---|---|---|---|---|---|---|---|
| Aspirin | 98,528 | 1,657 | 52.7 | 75.8 (8.6) | 30.8 | 21.6 | 30.0 | 16.9 | 0.6 | 32.7 | 30.1 | 61.6 |

Salsalate

| | | | | | | | | | | | | |
|---|---|---|---|---|---|---|---|---|---|---|---|---|
| Salsalate | 4,376 | 42 | 64.7 | 75.1 (7.9) | 11.9 | 32.6 | 31.4 | 23.5 | 0.6 | 14.5 | 19.8 | 44.4 |

Diflunisal

| | | | | | | | | | | | | |
|---|---|---|---|---|---|---|---|---|---|---|---|---|
| Diflunisal | 3,603 | 7 | 62.5 | 73.0 (6.9) | 41.0 | 18.5 | 32.1 | 7.5 | 0.9 | 17.5 | 21.5 | 51.1 |

CAD: coronary artery disease; T2D: type 2 diabetes; HTN: hypertension.

TABLE 3
Phenotype definitions (ICD9/ICD10 codes) for traumatic brain injury, Alzheimer's
disease, type 2 diabetes, hypertension, and coronary artery disease.

Traumatic brain injury
F07.81, S02.0XXA, S02.0XXB, S02.101A, S02.101B, S02.109A, S02.91XA, S02.91XB, S06.0X0A,
S06.0X1A, S06.0X9A, S06.1X0A, S06.1X1A, S06.1X3A, S06.1X9A, S06.330A, S06.331A,
S06.333A, S0.6339A, S06.360A, S06.361A, S06.363A, S06.369A, S06.370A, S06.371A,
S06.373A, S06.379A, S06.4X0A, S064.X1A, S06.4X3A, S06.4X9A, S06.5X0A, S06.5X1A,
S06.5X3A, S06.5X9A, S06.6X0A, S06.6X1A, S06.6X3A, S06.6X9A, S09.8XXA
Alzheimer's disease
3310, F00, F000A, F001A, F002A, F009A, G30, G300, G301, G308, G309
Type 2 diabetes
25000, 25050, 25002, 25052, 25010, 25060, 25012, 25062, 25020, 25070, 25022, 25072,
25030, 25080, 25032, 25082, 25040, 25090, 25042, 25092, E089, E1100, E1101, E1110,
E1121, E1122, E1129, E11311, E11319, E113219, E113291, E113292, E113293, E113299,
E113319, E113391, E113392, E113393, E113399, E113419, E113491, E113492, E113493,
E113499, E113519, E113591, E113592, E113593, E113599, E1136, E1139, E1140, E1142,
E1143, E1144, E1149, E1151, E1152, E1159, E11610, E11618, E11620, E11621, E11628,
E11630, E11641, E11649, E1165, E1169, E118, E119, E1300, E1310, E1311, E1322, E1329,
E1339, E1349, E1351, E13628, E1365, E1369, E138, E139
Hypertensions
4010, 4011, 4019, I10, I169
Coronary artery disease
410, 411, 412, 4131 414, I20, I21, I22, I23, I24, I25

Propensity score estimation. We define NE=north east, NC=north central, S=south, W=west, T2D=type 2 diabetes, HT=hypertension and CAD=coronary artery disease. The propensity score of taking a candidate drug vs. a comparator drug (i.e., aspirin) was estimated by the following logistic regression model:

$$logit [ Pr ( candidate drug = 1 ) ] = {β}_{0} + {β}_{1} Age + {β}_{2} Gender + {β}_{3} 1 ( Location = NE ) + {β}_{4} 1 ( Location = NC ) + {β}_{5} 1 ( Location = S ) + {β}_{6} 1 ( Location = W ) + {β}_{7} T 2 D + {β}_{8} HT + {β}_{9} CAD .$$

Survival analysis. Stratified Cox models were used to compare the TBI or AD risks. For a candidate drug (i.e., salsalate or diflunisal) versus a comparator drug (i.e., aspirin), the analyses were stratified (n strata=10) by the estimated propensity score. All analyses were stratified based on the subgroups defined by sex, T2D, HT and CAD diagnoses (n strata=10). Finally, propensity score stratified Cox-proportional hazards models were used to conduct statistical inference for the hazard ratios (HR) of developing AD or TBI between cohorts.

### REFERENCES

Allen, R.S., Motz, C.T., Feola, A., Chesler, K.C., Haider, R., Rao, S.S., Skelton, L.A., Fliesler, S.J., and Pardue, M.T. (2018). Long-term functional and structural consequences of primary blast overpressure to the eye. J. Neurotrauma 35, 2104-2116.
Cao, G., Xing, J., Xiao, X., Liou, A.K.F., Gao, Y., Yin, X-M, Clark, R.S.B., Graham, S.H., and Chen. J. (2007). Critical Role of Calpain I in Mitochondrial Release of Apoptosis-Inducing Factor in Ischemic Neuronal Injury. J. Neurosci. 27, 9278.
Cheng F, Desai RJ, Handy DE, Wang R, Schneeweiss S, Barabási AL, Loscalzo J. (2018) Network-based approach to prediction and population-based validation of in silico drug repurposing. Nat Commun. 12;9(1):2691.
Forrester, M.T., Thompson, J.W., Foster, M.W., Nogueira, L., Moseley, M.A., and Stamler, J.S. (2009) Proteomic analysis of S-nitrosylation and denitrosylation by resin-assisted capture. Nat. Biotechnol. 27, 557-559.
Kraus, A., Saijo, E., Metrick, M.A., Newell, K., Sigurdson, C.J., Zanusso, G., Ghetti, B., and Caughey, B. (2019). Seeding selectivity and ultrasensitive detection of tau aggregate conformers of Alzheimer disease. Acta Neuropathol. 137, 585-598.
Kristian, T. (2010). Isolation of Mitochondria from the CNS. Curr. Protoc. in Neurosci. 52, 7.22.21-27.22.12.
Min, S.W., Cho, S-Y., Zhou, Y., Schroeder, S., Haroutunian, V., Seeley, W.W., Huang, H.J., Shen, Y., Masliah, E., Mukherjee, C., et al. (2010). Acetylation of tau inhibits its degradation and contributes to tauopathy. Neuron 67, 953-966.
Newman, A.J., Hayes, S.H., Rao, A.S., Allman, B.L., Manohar, S., Ding, D., Stolzberg, D., Lobarinas, E., Mollendorf, J.C., and Salvi R. (2015). Low-cost blast wave generator for studies of hearing loss and brain injury: blast wave effects in closed spaces. J. Neurosci. Methods. 242, 82-92.
Ruifrok, A.C. and Johnston, D.A. (2001). Quantification of histochemical staining by color deconvolution. Anal. Quant. Cytol. Histol. 23, 291-299.
Schmittgen, T.D. and Livak, K.J. (2008). Analyzing real-time PCR data by the comparative C(T) method. Nat. Protoc. 3, 1101-1108.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A method of diagnosing and/or prognosing a neurodegenerative disease in a subject, the method comprising:
obtaining a plasma or blood sample from a subject; and
detecting a level of acetylated tau in the plasma or blood sample, wherein a level of acetylated tau that is at least 25% or 50% higher than a control level in a healthy subject indicates that the subject has a neurodegenerative disease such as traumatic brain injury.

2. The method of claim 1, wherein the obtaining step comprises obtaining a plasma sample from the subject.

3. The method of claim 2, further comprising depleting albumin and immunoglobulin from the plasma sample.

4. The method of claim 1, wherein the method does not involve any brain biopsy sample.

5. The method of claim 1, wherein the detecting step comprises using an antibody or antigen-binding fragment thereof that specifically binds acetylated tau.

6. The method of claim 5, wherein the antibody is a polyclonal antibody.

7. The method of claim 5, wherein the antibody is a monoclonal antibody.

8. A method of treating a neurodegenerative disease in a subject, the method comprising administering to a subject in need thereof a therapeutically effective amount of an agent that blocks GAPDH S-nitrosylation, inhibits p300/CBP activity, and/or enhances Sirtuin1 activity, whereby accumulation of ac-tau in brain and/or plasma in the subject is reduced.

9. The method of claim 8, comprising administering a therapeutically effective amount of an inhibitor of GAPDH nitrosylation such as CGP3466B (Omigapil) to the subject.

10. The method of claim 8, comprising administering a therapeutically effective amount of p300/CBP inhibitor such as salsalate and/or diflunisal to the subject.

11. The method of claim 10, wherein the salsalate and/or diflunisal is administered at a low, non-anti-neuroinflammatory dose, wherein said dose is 50% or less than an anti-neuroinflammatory dose

12. The method of claim 11, wherein the low, non-anti-neuroinflammatory dose is about 10-25 mg/kg/day.

13. The method of claim 11, wherein the anti-neuroinflammatory dose is about 50 mg/kg/day.

14. The method of claim 9, further comprising co-administering an effective amount of 3,6-dibromo-β-fluoro-N-(3-methoxyphenyl)-9H-carbazole-9-propanamine to the subject.

15. The method of claim 8, comprising administering an effective amount of 3,6-dibromo-β-fluoro-N-(3-methoxyphenyl)-9H-carbazole-9-propanamine to the subject.

16. The method of claim 1, wherein the neurodegenerative disease is selected from subarachnoid hemorrhage, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

17. An apparatus for diagnosing and/or prognosing a neurodegenerative disease in a subject, comprising:
a support material; and
an antibody or antigen-binding fragment thereof that specifically binds acetylated tau, wherein said antibody or antigen-binding fragment thereof is adsorbed in or associated with the support material.

18. The apparatus of claim 17, wherein the antibody is a polyclonal antibody.

19. The apparatus of claim 17, wherein the antibody is a monoclonal antibody.

20. The method of claim 8, wherein the neurodegenerative disease is selected from subarachnoid hemorrhage, schizophrenia, major depression, bipolar disorder, normal aging, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy, chronic stress, abuse of a neuro-active drug, retinal degeneration, spinal cord injury, peripheral nerve injury, physiological weight loss associated with various conditions, cognitive decline and/or general frailty associated with normal aging and/or chemotherapy, chemotherapy induced neuropathy, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), spinal cord injury, traumatic spinal cord injury, peripheral nerve injury (such as peripheral nerve crush injury, neonatal brachial plexus palsy, and traumatic facial nerve palsy), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Multiple sclerosis, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.
